(19) United States
(12) Patent Application Publication
(10) Pub. No.: US 2015/0105377 A1
(43) Pub. Date: Apr. 16, 2015

(54) Methods and Compositions for RAF Kinase Mediated Diseases

(71) Applicant: ARIAD Pharmaceuticals, Inc., Cambridge, MA (US)

(72) Inventors: Joseph M. Gozgit, Framingham, MA (US); Victor M. Rivera, Arlington, MA (US); William C. Shakespeare, Southborough, MA (US); Xiaotian Zhu, Newton, MA (US); David C. Dalgarno, Brookline, MA (US)

(21) Appl. No.: 14/396,287

(22) PCT Filed: Mar. 12, 2013
(86) PCT No.: PCT/US13/30444

§ 371 Date: Oct. 22, 2014

Related U.S. Application Data
(60) Provisional application No. 61/638,247, filed on Apr. 25, 2012.
(60) Provisional application No. 61/776,535, filed on Mar. 11, 2013.

(51) Int. Cl. C07D 487/04; C07D 473/34; C07D 471/04

(52) U.S. Cl. CPC C07D 487/04; C07D 471/04; C07D 473/34
USPC 514/218; 514/249; 514/300; 514/253.04; 514/248; 514/233.2; 514/245; 514/252.16; 514/263.2; 514/259.3

(57) ABSTRACT

The invention discloses methods and compositions for treating or preventing RAF kinase mediated diseases or conditions by administering a compound of Formula 1: or a pharmaceutically acceptable salt, solvate or hydrate thereof, wherein the variables are defined as herein.

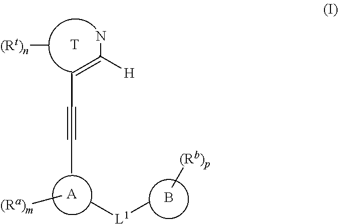

### TECHNICAL FIELD

This invention relates to methods and compositions for treating or preventing RAF kinase mediated diseases or conditions by administering a RAF inhibitor disclosed herein or a pharmaceutically acceptable salt thereof.

### BACKGROUND

The family of Raf kinases includes three serine/threonine specific protein kinases known as A-Raf, B-Raf and C-Raf. The acronym RAF stands for Rapidly Accelerated Fibrosarcoma. Zebisch, A., et al., Cellular and Molecular Life Sciences, 63(11): 1314-1330 (2006).

The RAS-RAF-MEK-ERK or MAPK signaling pathway drives cell proliferation and survival and is commonly activated in human cancers. Inhibition of Raf kinase has been implicated in the treatment of a variety of diseases or disorders including hematological cancers such as acute myeloid leukemia and solid tumors such as melanoma, medullary thyroid cancer, carcinoid, small cell lung cancer and pheochromocytoma. Crump., M., Curr. Pharm. Design, 8(25):2243-8 (2002); Kunnimalaiyaan, M. and Chen, H., et al., Anticancer Drugs., 17(2): 139-42 (2006).

Ninety percent of activating BRAF mutations occur at valine 600 (V600), and this alteration is found in approximately 7% of human cancers, including 60% of melanomas, 10-15% of colorectal cancers, and 30-70% of papillary thyroid carcinomas. Davies, et al., Nature, 417, 949-954 (2002); Kimura, et al., Cancer Research, 63, 1454-1457 (2003). ZELBORAF® (vemurafenib) is a potent selective BRAF V600E inhibitor that has been shown to cause partial or complete responses in 80% of patients with metastatic melanoma carrying the V600E mutation. Flaherty, et al., NEJM, 363, 809-819 (2010). It was approved by the US FDA in 2011 and is currently indicated for the treatment of patients with unresectable or metastatic melanoma with BRAF^{V600E} mutation as detected by an FDA approved test.

Despite its approval, ZELBORAF has its limitations. For example, it is not recommended for use in patients with wild-type BRAF melanoma. ZELBORAF full prescribing information dated August 2011. In addition, response rates to vemurafenib are relatively poor (˜5%) in BRAF mutant colorectal cancers. Kopetz, et al., J Clin Oncol, 28, abstract 3534 (2010). Several mechanisms of resistance to specific BRAF inhibitors have been raised. Recent studies have shown that resistance can be mediated through BRAF amplification and through paradoxical feedback activation of RAF signaling in cancers with active RAS. Corcoran, et al., Science Signaling, 3, ra84 (2010); Poulikakos, et al., Nature, 464, 427-430 (2010); Heidorn, et al., Cell, 140, 209-221 (2010).

Applicant's own WO 2007/075869, which is hereby incorporated herein by reference for all purposes, discloses certain compounds that inhibit inter alia Abl. The applicability of such Abl inhibitors to RAF inhibition may possibly be explained by the findings that c-RAF-1 enzymatic activity is regulated by Bcr-Abl. Skorski, T., et al., Cancer Research, 55, 2275-2278 (1995). Applicant's own WO 2011/053938, which is hereby incorporated herein by reference for all purposes, discloses that these compounds have a wide range of kinase activity beyond the initial focus on Abl inhibition. For instance, these compounds demonstrate potency against PDGFR, c-SRC, and certain other kinases shown at Table 8. One notable Abl inhibitor is ponatinib, which is currently the subject of a clinical trial to determine the efficacy of ponatinib in patients with chronic myeloid leukemia (CML) in chronic phase (CP), accelerated phase (AP) or blast phase (BP) or with Ph positive (Ph+) acute lymphoblastic leukemia (ALL) who either are resistant or intolerant to either dasatinib or nilotinib, or have the T315I mutation of Bcr-Abl (clinical trials.gov identifier NCT01207440). ICLUSIG® (ponatinib) was approved by the US FDA in December 2012 for the treatment of adult patients with chronic phase, accelerated phase, or blast phase chronic myeloid leukemia (CML) that is resistant or intolerant to prior tyrosine kinase inhibitor therapy or Philadelphia chromosome positive acute lymphoblastic leukemia (Ph+ALL) that is resistant or intolerant to prior tyrosine kinase inhibitor therapy. Neither WO 2007/075869 nor WO 2011/053938 explicitly mentions that such Abl inhibitors are active against RAF.

### SUMMARY

It has been unexpectedly discovered that certain Abl inhibitors are also potent inhibitors of RAF, including ARAF, BRAF, and CRAF and mutants thereof and accordingly are potentially useful for the treatment or prevention of certain diseases or disorders mediated by RAF.

In one aspect, this disclosure provides methods for treating or preventing a RAF kinase mediated disease or condition in a subject in need thereof by administering to the subject an effective amount of a RAF inhibitor, wherein the RAF inhibitor is a compound of Formula I:

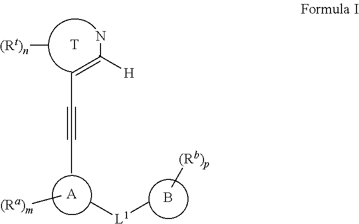

or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;

or a pharmaceutically acceptable salt, solvate or hydrate thereof.

In certain embodiments, the present disclosure provides a method for treating or preventing an A-RAF kinase mediated disease or condition in a subject in need thereof comprising administering to the subject an effective amount of a compound disclosed herein. In certain of these embodiments, the compound is a selective A-RAF inhibitor.

In certain embodiments, the present disclosure provides a method for treating or preventing an B-RAF kinase mediated disease or condition in a subject in need thereof comprising administering to the subject an effective amount of a compound disclosed herein. In certain of these embodiments, the compound is a selective B-RAF inhibitor.

In certain embodiments, the present disclosure provides a method for treating or preventing an C-RAF kinase mediated disease or condition in a subject in need thereof comprising administering to the subject an effective amount of a compound disclosed herein. In certain of these embodiments, the compound is a selective C-RAF inhibitor.

In another aspect, this disclosure provides pharmaceutical compositions for treating or preventing a RAF kinase mediated disease or condition in a subject in need thereof comprising an effective amount of a RAF inhibitor, wherein the RAF inhibitor is a compound of Formula I:

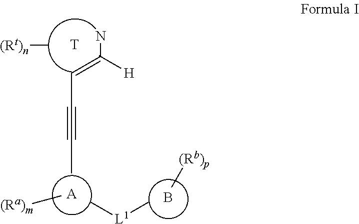

or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;

or a pharmaceutically acceptable salt, solvate or hydrate thereof; and

a pharmaceutically acceptable carrier.

In another aspect, this disclosure provides kits including: (a) a presently disclosed RAF inhibitor, and (b) instructions for administering the compound to a subject diagnosed with or at risk of developing a RAF kinase mediated disease or condition. The RAF inhibitor can be formulated for administration according to any of the dosing regimens described herein. As noted at the outset, the RAF inhibitor used in the various embodiments of the invention may be in the form of its free base or a pharmaceutically acceptable salt thereof.

In another aspect, this disclosure provides a method for inhibiting a RAF kinase in a subject by administering to the subject an effective amount of a presently disclosed compound of Formula I. In certain embodiments, the subject has an aberrant RAF kinase, such as B-RAF^{V600E} or B-RAF^{V600K}.

In another aspect, this disclosure provides a compound for use in a method to treat or prevent a RAF kinase mediated disease or condition in a subject in need thereof, wherein the compound is a presently disclosed compound of Formula I.

In certain embodiments of any of the foregoing methods or pharmaceutical compositions in the compound of Formula I, the RAF inhibitor is a compound selected from the group consisting of:
N-(3-(1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyrazin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyridin-3-ylethynyl)-4-methyl-N-(3-(4-methyl-1H-imidazol-1-yl)-5-(trifluoromethyl)phenyl)benzamide;
N-(3-(1H-imidazol-1-yl)-5-(trifluoromethyl)phenyl)-3-(imidazo[1,2-a]pyridin-3-ylethynyl)-4-methylbenzamide;
3-(Imidazo[1,2-a]pyridin-3-ylethynyl)-4-methyl-N-(4-(trifluoromethyl)pyridin-2-yl)benzamide;
N-(5-tert-butylisoxazol-3-yl)-3-(imidazo[1,2-a]pyridin-3-ylethynyl)-4-methylbenzamide;
3-(Imidazo[1,2-a]pyridin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide;
3-((8-Acetamidoimidazo[1,2-a]pyridin-3-yl)ethynyl)-4-methyl-N-(4-(trifluoromethyl)pyridin-2-yl)benzamide;
N-(3-(1H-imidazol-1-yl)-5-(trifluoromethyl)phenyl)-3-((8-acetamidoimidazo[1,2-a]pyridin-3-yl)ethynyl)-4-methylbenzamide;
4-Methyl-3-((8-(4-(methylsulfonyl)phenylamino)imidazo[1,2-a]pyridin-3-yl)ethynyl)-N-(4-(trifluoromethyl)pyridin-2-yl)benzamide;
4-methyl-3-((8-(4-sulfamoylphenylamino)imidazo[1,2-a]pyridin-3-yl)ethynyl)-N-(4-(trifluoromethyl)pyridin-2-yl)benzamide;
(R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide;
N-(3-(Imidazo[1,2-b]pyridazin-3-ylethynyl)-4-methylphenyl)-4-((4-methylpiperazin-1-yl)methyl)-3-(trifluoromethyl)benzamide;
3-(Imidazo[1,2-b]pyridazin-3-ylethynyl)-4-methyl-N-(4-((4-methylpiperazin-1-yl)methyl)-3-(trifluoromethyl)phenyl)benzamide;
N-(3-Chloro-4-((4-methylpiperazin-1-yl)methyl)phenyl)-3-(imidazo[1,2-b]pyridazin-3-ylethynyl)-4-methylbenzamide;
N-(3-Cyclopropyl-4-((4-methylpiperazin-1-yl)methyl)phenyl)-3-(imidazo[1,2-b]pyridazin-3-ylethynyl)-4-methylbenzamide;
3-(Imidazo[1,2-b]pyridazin-3-ylethynyl)-N-(4-((4-methylpiperazin-1-yl)methyl)-3-(trifluoromethyl)phenyl)benzamide;
N-(4-((4-(2-Hydroxyethyl)piperazin-1-yl)methyl)-3-(trifluoromethyl)phenyl)-3-(imidazo[1,2-b]pyridazin-3-ylethynyl)-4-methylbenzamide; and
3-(Imidazo[1,2-b]pyridazin-3-ylethynyl)-4-methyl-N-(4-(piperazin-1-ylmethyl)-3-(trifluoromethyl)phenyl)benzamide,
   or a pharmaceutically acceptable salt thereof.

Additional features and advantages of the methods and pharmaceutical compositions disclosed herein will be apparent from the following detailed description.

### BRIEF DESCRIPTION OF THE DRAWINGS

FIG. 1A, shows the activity of ponatinib and vemurafenib in the A375 BRAF^{V600E} melanoma cell line. The concentration (nM) of each inhibitor is plotted against percent growth inhibition. In FIG. 1B, A375 cells were treated for 1 hour with the indicated concentrations of ponatinib. Cell lysates were immunoblotted to detect the indicated proteins.

FIG. 2A, shows the activity of ponatinib, sorafenib, and vemurafenib in the SH-4 BRAF^{V600E} melanoma cell line. The concentration (nM) of each inhibitor is plotted against percent growth inhibition. In FIG. 2B, SH-4 cells were treated for 1 hour with the indicated concentrations of ponatinib or vemurafenib. Cell lysates were immunoblotted to detect the indicated proteins.

FIG. 3 shows the activity of ponatinib and vemurafenib in the SK-MEL-24 BRAF^{V600E} melanoma cell line. The concentration (nM) of each inhibitor is plotted against percent growth inhibition.

FIG. 4 A, shows the activity of ponatinib, sorafenib, and vemurafenib in the HT-29 BRAF^{V600E} colorectal cancer cell line. The concentration (nM) of each inhibitor is plotted against percent growth inhibition. In FIG. 4B, HT-29 cells were treated for 1 hour with the indicated concentrations of ponatinib, sorafenib, or vemurafenib. Cell lysates were immunoblotted to detect the indicated proteins.

### DETAILED DESCRIPTION

### Definitions

In reading this document, the following information and definitions apply unless otherwise indicated.

As used herein, the term “ponatinib” means 3-(imidazo[1,2-b]pyridazin-3-ylethynyl)-4-methyl-N-(4-((4-methylpiperazin-1-yl)-methyl-3-(trifluoromethyl)phenyl)benzamide (as shown in Example 16 herein) and having the chemical structure depicted below:

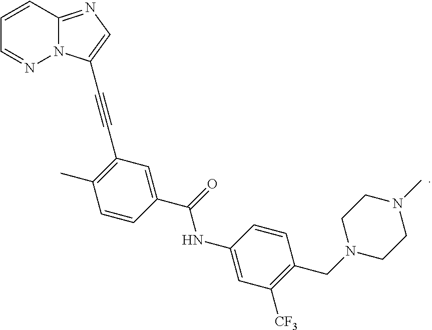 [SMILES: Cc1ccc(C(=O)Nc2ccc(CN3CCN(C)CC3)c(C(F)(F)F)c2)cc1C#Cc1cnc2cccnn12]

The term ponatinib refers only to its free base unless a pharmaceutically acceptable salt (such as ponatinib HCl) is explicitly mentioned.

As used herein, the term “mean steady state trough concentration” means the average plasma concentration of a compound disclosed herein observed for a group of subjects as part of a dosing regimen for a therapy of the invention administered over a period of time sufficient to produce steady state pharmacokinetics (e.g., a period of 23 days of daily dosing), wherein the mean trough concentration is the average circulating concentration over all of the subjects at a time just prior to (i.e., within 1 hour of) the next scheduled administration in the regimen (e.g., for a daily regimen the trough concentration is measured about 24 hours after an administration of a compound disclosed herein and just prior to the subsequent daily administration).

As used herein, the terms “administration” or “administering” mean a route of administration for a compound disclosed herein. Exemplary routes of administration include, but are not limited to, oral, intravenous, intraperitoneal, intraarterial, and intramuscular. The preferred route of administration can vary depending on various factors, e.g., the components of the pharmaceutical composition comprising a compound disclosed herein, site of the potential or actual disease and severity of disease. While ponatinib will generally be administered per orally, other routes of administration can be useful in carrying out the methods of the invention.

As used herein, the term “unit dosage form” means a physically discrete unit containing a predetermined quantity of a compound disclosed herein that is suitable for administration. Exemplary unit dosage forms include, but are not limited to, a pill, tablet, caplet, hard capsule or soft capsule.

As used herein, the term “pharmaceutically acceptable salt” means salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, phosphonates and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the isolation and purification of the compounds of the invention, or separately by reacting the free base or free acid of a compound of the invention with a suitable base or acid, respectively. Examples of pharmaceutically acceptable, nontoxic acid addition salts of a compound disclosed herein are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methane-sulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “pharmaceutically acceptable carrier” or “pharmaceutically acceptable adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound. Pharmaceutically acceptable carriers, adjuvants and vehicles that can be used in the pharmaceutical compositions of this invention include, but are not limited to, ion exchangers, alumina, aluminum stearate, lecithin, self emulsifying drug delivery systems (SEDDS) such as d-atocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as u-, P-, and y-cyclodextrin, or chemically modified derivatives such as hydroxyalkylcyclodextrins, including 2 and 3-hydroxypropyl-cyclodextrins, or other solubilized derivatives may also be advantageously used to enhance delivery of compounds of the formulae described herein.

As used herein, the terms “treatment” or “treating” mean: (1) improving or stabilizing the subject's condition or disease or (2) preventing or relieving the development or worsening of symptoms associated with the subject's condition or disease.

As used herein, the terms “amount effective” or “effective amount” mean the amount of a compound disclosed herein that when administered to a subject for treating a disease, is sufficient to effect such treatment of the disease. Any improvement in the patient is considered sufficient to achieve treatment. An effective amount of a compound disclosed herein, used for the treatment of a RAF kinase mediated disease or condition can vary depending upon the manner of administration, the age, body weight, and general health of the patient. Ultimately, the prescribers or researchers will decide the appropriate amount and dosage regimen.

As used herein, the term “RAF kinase mediated disease or condition” means a disease or condition in which the biological function of a RAF kinase (defined immediately below), including any mutations thereof, affects the development and/or course of the disease or condition, and/or in which modulation of the RAF kinase alters the development, course, and/or symptoms of the disease or condition. A RAF kinase mediated disease or condition includes a disease or condition for which RAF inhibition provides a therapeutic benefit, e.g. wherein treatment with a RAF inhibitor, including a compound described herein, provides a therapeutic benefit to the subject suffering from or at risk of the disease or condition. Exemplary diseases or conditions that are mediated by RAF include, but are not limited to, certain hematological cancers including acute myeloid leukemia and solid tumors such as melanoma, colorectal cancer, medullary thyroid cancer, carcinoid, small cell lung cancer and pheochromocytoma. Subtypes of these disorders or conditions are also included within the definition of “RAF kinase mediated disease or condition”. For example, a subtype of melanoma is B-RAF^{V600E} mutation-positive metastatic melanoma.

As used herein, the terms “RAF kinase” or simply “RAF” includes, but is not limited to, A-RAF, mutations of A-RAF, B-RAF, mutations of B-Raf, C-RAF or c-RAF-1 and mutations of C-RAF or c-RAF-1. An exemplary B-RAF mutation is V600E. Another exemplary B-RAF mutation is V600K.

As used herein, the terms “subject” and “patient” are used herein interchangeably. They refer to a human or another mammal (e.g., mouse, rat, rabbit, dog, cat, cattle, swine, sheep, horse or primate) that can be afflicted with or is susceptible to a disease or disorder but may or may not have the disease or disorder. In certain embodiments, the subject is a human being.

As used herein, the term “alkyl” is intended to include linear (i.e., unbranched or acyclic), branched, cyclic, or polycyclic non aromatic hydrocarbon groups, which are optionally substituted with one or more functional groups. Unless otherwise specified, “alkyl” groups contain one to eight, and preferably one to six carbon atoms. C₁₋₆ alkyl, is intended to include C₁, C₂, C₃, C₄, C₅, and C₆ alkyl groups. Lower alkyl refers to alkyl groups containing 1 to 6 carbon atoms. Examples of Alkyl include, but are not limited to, methyl, ethyl, n-propyl, isopropyl, cyclopropyl, butyl, isobutyl, sec-butyl, tert-butyl, cyclobutyl, pentyl, isopentyl tert-pentyl, cyclopentyl, hexyl, isohexyl, cyclohexyl, etc. Alkyl may be substituted or unsubstituted. Illustrative substituted alkyl groups include, but are not limited to, fluoromethyl, difluoromethyl, trifluoromethyl, 2-fluoroethyl, 3-fluoropropyl, hydroxymethyl, 2-hydroxyethyl, 3-hydroxypropyl, benzyl, substituted benzyl, phenethyl, substituted phenethyl, etc.

As used herein, the term “Alkoxy” means a subset of alkyl in which an alkyl group as defined above with the indicated number of carbons attached through an oxygen bridge. For example, “alkoxy” refers to groups —O-alkyl, wherein the alkyl group contains 1 to 8 carbons atoms of a linear, branched, cyclic configuration. Examples of “alkoxy” include, but are not limited to, methoxy, ethoxy, n-propoxy, i-propoxy, t-butoxy, n-butoxy, s-pentoxy and the like.

As used herein, the term “Haloalkyl” is intended to include both branched and linear chain saturated hydrocarbon having one or more carbon substituted with a Halogen. Examples of haloalkyl, include, but are not limited to, trifluoromethyl, trichloromethyl, pentafluoroethyl and the like.

As used herein, the term “alkenyl” is intended to include hydrocarbon chains of linear, branched, or cyclic configuration having one or more unsaturated Carbon-carbon bonds that may occur in any stable point along the chain or cycle. Unless otherwise specified, “alkenyl” refers to groups usually having two to eight, often two to six carbon atoms. For example, “alkenyl” may refer to prop-2-enyl, but-2-enyl, but-3-enyl, 2-methylprop-2-enyl, hex-2-enyl, hex-5-enyl, 2,3-dimethylbut-2-enyl, and the like. Furthermore, alkenyl groups may be substituted or unsubstituted.

As used herein, the term “alkynyl” is intended to include hydrocarbon chains of either linear or branched configuration, having one or more carbon-carbon triple bond that may occur in any stable point along the chain. Unless otherwise specified, “alkynyl” groups refer refers to groups having two to eight, preferably two to six carbons. Examples of “alkynyl” include, but are not limited to prop-2-ynyl, but-2-ynyl, but-3-ynyl, pent-2-ynyl, 3-methylpent-4-ynyl, hex-2-ynyl, hex-5-ynyl, etc. Furthermore, alkynyl groups may be substituted or unsubstituted.

As used herein, the term “Cycloalkyl” is a subset of alkyl and includes any stable cyclic or polycyclic hydrocarbon groups of from 3 to 13 carbon atoms, any of which is saturated. Examples of such cycloalkyl include, but are not limited to cyclopropyl, norbornyl, [2.2.2]bicyclooctane, [4.4.0]bicyclodecane, and the like, which, as in the case of other alkyl moieties, may optionally be substituted. The term “cycloalkyl” may be used interchangeably with the term “carbocycle”.

As used herein, the term “Cycloalkenyl” is a subset of alkenyl and includes any stable cyclic or polycyclic hydrocarbon groups of from 3 to 13 carbon atoms, preferably from 5 to 8 carbon atoms, which contains one or more unsaturated carbon-carbon double bonds that may occur in any point along the cycle. Examples of such cycloalkenyl include, but are not limited to cyclopentenyl, cyclohexenyl and the like.

As used herein, the term “Cycloalkynyl” is a subset of alkynyl and includes any stable cyclic or polycyclic hydrocarbon groups of from 5 to 13 carbon atoms, which contains one or more unsaturated carbon-carbon triple bonds that may occur in any point along the cycle. As in the case of other alkenyl and alkynyl moieties, cycloalkenyl and cycloalkynyl may optionally be substituted.

As used herein, the terms “Heterocycle”, “heterocyclyl”, or “heterocyclic” as used herein refers to non-aromatic ring systems having five to fourteen ring atoms, preferably five to ten, in which one or more ring carbons, preferably one to four, are each replaced by a heteroatom such as N, O, or S. Non-limiting examples of heterocyclic rings include 3-1H-benzimidazol-2-one, (1-substituted)-2-oxo-benzimidazol-3-yl, 2-tetrahydrofuranyl, 3-tetrahydrofuranyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocyclyl” or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocycle”, “heterocyclyl”, or “heterocyclic” whether saturated or partially unsaturated, also refers to rings that are optionally substituted.

As used herein, the term “aryl” used alone or as part of a larger moiety as in “aralkyl”, “aralkoxy”, or “aryloxy-alkyl”, refers to aromatic ring groups having six to fourteen ring atoms, such as phenyl, 1-naphthyl, 2-naphthyl, 1-anthracyl and 2-anthracyl. An “aryl” ring may contain one or more substituents. The term “aryl” may be used interchangeably with the term “aryl ring”. “Aryl” also includes fused polycyclic aromatic ring systems in which an aromatic ring is fused to one or more rings. Non-limiting examples of useful aryl ring groups include phenyl, hydroxyphenyl, halophenyl, alkoxyphenyl, dialkoxyphenyl, trialkoxyphenyl, alkylenedioxyphenyl, naphthyl, phenanthryl, anthryl, phenanthro and the like, as well as 1-naphthyl, 2-naphthyl, 1-anthracyl and 2-anthracyl. Also included within the scope of the term “aryl”, as it is used herein, is a group in which an aromatic ring is fused to one or more non-aromatic rings, such as in a indanyl, phenanthridinyl, or tetrahydronaphthyl, where the radical or point of attachment is on the aromatic ring.

As used herein, the term “heteroaryl” as used herein refers to stable heterocyclic, and polyheterocyclic aromatic moieties having 5-14 ring atoms. Heteroaryl groups may be substituted or unsubstituted and may comprise one or more rings. Examples of typical heteroaryl rings include 5-membered monocyclic ring groups such as thienyl, pyrrolyl, imidazolyl, pyrazolyl, furyl, isothiazolyl, furazanyl, isoxazolyl, thiazolyl and the like; 6-membered monocyclic groups such as pyridyl, pyrazinyl, pyrimidinyl, pyridazinyl, triazinyl and the like; and polycyclic heterocyclic ring groups such as benzo[b]thienyl, naphtho[2,3-b]thienyl, thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxalinyl, quinazolinyl, benzothiazole, benzimidazole, tetrahydroquinoline cinnolinyl, pteridinyl, carbazolyl, beta-carbolinyl, phenanthridinyl, acridinyl, perimidinyl, phenanthrolinyl, phenazinyl, isothiazolyl, phenothiazinyl, phenoxazinyl, and the like (see e.g. Katritzky, Handbook of Heterocyclic Chemistry). Further specific examples of heteroaryl rings include 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indolyl, isoindolyl, acridinyl, or benzoisoxazolyl. Heteroaryl groups further include a group in which a heteroaromatic ring is fused to one or more aromatic or nonaromatic rings where the radical or point of attachment is on the heteroaromatic ring. Examples include tetrahydroquinoline, tetrahydroisoquinoline, and pyrido[3,4-d]pyrimidinyl, imidazo[1,2-a]pyrimidyl, imidazo[1,2-a]pyrazinyl, imidazo[1,2-a]pyiridinyl, imidazo[1,2-c]pyrimidyl, pyrazolo[1,5-a][1,3,5]triazinyl, pyrazolo[1,5-c]pyrimidyl, imidazo[1,2-b]pyridazinyl, imidazo[1,5-a]pyrimidyl, pyrazolo[1,5-b][1,2,4]triazine, quinolyl, isoquinolyl, quinoxalyl, imidazotriazinyl, pyrrolo[2,3-d]pyrimidyl, triazolopyrimidyl, pyridopyrazinyl. The term “heteroaryl” also refers to rings that are optionally substituted. The term “heteroaryl” may be used interchangeably with the term “heteroaryl ring” or the term “heteroaromatic”.

### Methods

As discussed herein, this disclosure provides a method for treating or preventing a RAF kinase mediated disease or condition in a subject in need thereof by administering to the subject an effective amount of a RAF inhibitor, wherein the RAF inhibitor is a compound of Formula I:

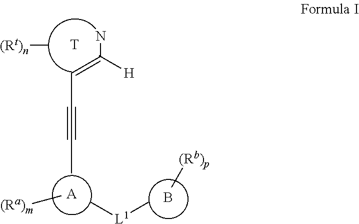

or a tautomer, or an individual isomer or a mixture of isomers thereof wherein:

Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;

Ring A is a 5- or 6-membered aryl or heteroaryl ring and is optionally substituted with 1-4 R^{a} groups;

Ring B is a 5- or 6-membered aryl or heteroaryl ring;

L¹ is selected from NR¹C(O), C(O)NR¹, NR¹C(O)O, NR¹C(O)NR¹, and OC(O)NR¹;

each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;

R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;

R¹, R² and R³ are independently selected from H, alkyl, alkenyl, alkynyl, cycloalkyl, cycloalkenyl, cycloalkynyl, aryl, heterocyclic and heteroaryl;

alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;

each occurrence of R⁴ is independently selected from alkyl, alkenyl, alkynyl, cycloalkyl, cycloalkenyl, cycloalkynyl, aryl, heterocyclic and heteroaryl;

each of the alkyl, alkenyl, alkynyl, cycloalkyl, cycloalkenyl, cycloalkynyl, aryl, heterocyclic and heteroaryl moieties is optionally substituted;

m is 0, 1, 2, 3 or 4;

n is 2 or 3;

p is 0, 1, 2, 3, 4 or 5; and,

r is 0, 1 or 2;

or a pharmaceutically acceptable salt, solvate or hydrate thereof.

In certain embodiments, the RAF inhibitor is ponatinib or a pharmaceutically acceptable salt thereof. In certain of these embodiments, the RAF inhibitor is ponatinib hydrochloride.

In certain embodiments, the RAF kinase mediated disease or condition treated or prevented with a compound of Formula I is a hematological cancer that is known to be implicated by the inhibition of a RAF tyrosine kinase such as acute myeloid leukemia (AML). In alternative embodiments, the RAF kinase mediated disease or condition treated or prevented with a compound of Formula I is a solid tumor that is known to be implicated by the inhibition of a RAF tyrosine kinase such as melanoma, colorectal cancer, medullary thyroid cancer, carcinoid, small cell lung cancer and pheochromocytoma. In certain embodiments, the RAF kinase mediated disease or condition treated or prevented with a compound of Formula I is a cancer that is implicated by a mutation or genetic aberration of a RAF tyrosine kinase. In further embodiments, the disease or condition is a cancer that is amenable to treatment by an inhibitor of the V600E mutant B-RAF.

In certain embodiments, this disclosure provides a method for treating or preventing an A-RAF kinase mediated disease or condition in a subject in need thereof by administering to the subject an effective amount of an A-RAF inhibitor, wherein the A-RAF inhibitor is a compound of Formula I as disclosed herein.

In certain embodiments, this disclosure provides a method for treating or preventing a B-RAF kinase mediated disease or condition in a subject in need thereof by administering to the subject an effective amount of a B-RAF inhibitor, wherein the B-RAF inhibitor is a compound of Formula I as disclosed herein.

Target kinase B-RAF (i.e., v-RAF murine sarcoma viral oncogene homolog B1) is a 84.4 kDa serine/threonine kinase encoded by chromosome 7q34 (symbol: BRAF). The mature protein comprises RBD (i.e., Ras binding domain), C1 (i.e., protein kinase C conserved region 1) and STK (i.e., serine/threonine kinase) domains.

Target kinase B-RAF is involved in the transduction of mitogenic signals from the cell membrane to the nucleus and may play a role in the postsynaptic responses of hippocampal neurons. As such, genes of the RAF family encode kinases that are regulated by Ras and mediate cellular responses to growth signals. Indeed, B-RAF kinase is a key component of the RAS→RAF→MEK→ERK/MAP kinase signaling pathway, which plays a fundamental role in the regulation of cell growth, division and proliferation, and, when constitutively activated, causes tumorigenesis. Among several isoforms of RAF kinase, the B-type, or B-RAF, is the strongest activator of the downstream MAP kinase signaling.

The BRAF gene is frequently mutated in a variety of human tumors, especially in malignant melanoma and colon carcinoma. The most common reported mutation was a missense thymine (T) to adenine (A) transversion at nucleotide 1796 (T1796A; amino acid change in the B-RAF protein is Val<600> to Glu<600>) observed in 80% of malignant melanoma tumors. Functional analysis reveals that this transversion is the only detected mutation that causes constitutive activation of B-RAF kinase activity, independent of RAS activation, by converting B-Raf into a dominant transforming protein.

Niihori et al., report that in 43 individuals with cardio-facio-cutaneous (CFC) syndrome, they identified two heterozygous KRAS mutations in three individuals and eight BRAF mutations in 16 individuals, suggesting that dysregulation of the RAS-RAF-ERK pathway is a common molecular basis for the three related disorders (Niihori et al., Nat. Genet., 38(3): 294-6 (2006).

In certain embodiments, this disclosure provides a method for treating or preventing a C-RAF kinase mediated disease or condition in a subject in need thereof by administering to the subject an effective amount of a C-RAF inhibitor, wherein the C-RAF inhibitor is a compound of Formula I as disclosed herein. In certain embodiments, the C-RAF (or c-RAF-1) kinase mediated disease is selected from colorectal, ovarian, lung and renal cell carcinoma, acute myeloid leukemia, myelodysplastic syndromes, tumor angiogenesis, and neuroendocrine tumors such as medullary thyroid cancer, carcinoid, small cell lung cancer and pheochromocytoma.

Target kinase c-Raf-1 (i.e., v-RAF murine sarcoma viral oncogene homolog 1) is a 73.0 kDa STK encoded by chromosome 3p25 (symbol: RAF1). c-RAF-1 can be targeted to the mitochondria by BCL2 (i.e., oncogene B-cell leukemia 2) which is a regulator of apoptotic cell death. Active c-RAF-1 improves BCL2-mediated resistance to apoptosis, and c-RAF-1 phosphorylates BAD (i.e., BCL2-binding protein). c-RAF-1 is implicated in carcinomas, including colorectal, ovarian, lung and renal cell carcinoma. C-RAF-1 is also implicated as an important mediator of tumor angiogenesis (Hood, J. D. et al., Science 296, 2404 (2002). C-Raf-1 inhibitors may also be useful for the treatment of acute myeloid leukemia and myelodysplastic syndromes (Crump, Curr Pharm Des, 8(25):2243-8 (2002). RAF-1 activators may be useful as treatment for neuroendocrine tumors, such as medullary thyroid cancer, carcinoid, small cell lung cancer and pheochromocytoma (Kunnimalaiyaan et al., Anticancer Drugs, 17(2):139-42 (2006). C-RAF-1 inhibitors may be useful in treating colorectal, ovarian, lung and renal cell carcinoma, acute myeloid leukemia, myelodysplastic syndromes, tumor angiogenesis, and neuroendocrine tumors such as medullary thyroid cancer, carcinoid, small cell lung cancer and pheochromocytoma.

In certain embodiments, the present disclosure provides for the use of an effective amount of a compound of Formula I for the preparation of a medicament for treating or preventing a RAF kinase mediated disease or condition. In related embodiments, the present disclosure provides a pharmaceutical composition for use in a method to treat or prevent a RAF kinase mediated disease or condition, wherein the pharmaceutical composition comprises a compound of Formula I or a pharmaceutically acceptable salt thereof and a pharmaceutically acceptable carrier.

Further to any of the above mentioned embodiments, a compound of the invention may also inhibit the effects of a mutation of the kinase, including, but not limited to, a mutation that is related to a disease state, such as a cancer. For example, B-Raf V600E mutant is present in a high percentage of some cancers, such as melanoma, and compounds of the invention will inhibit the kinase activity of this mutant.

Further to any of the above embodiments, a presently disclosed RAF inhibitor, i.e., a presently disclosed compound that inhibits one or more RAF kinases, may selectively inhibit one kinase relative to one or more other kinases, where preferably inhibition is selective with respect to any of the other kinases, whether a kinase discussed herein, or other kinases. In some embodiments, the compound may selectively inhibit the effects of a mutation of the kinase relative to the wild type kinase, for example B-RAF V600E relative to wild type B-RAF. Selective inhibition of one kinase relative to another is such that the IC50 for the one kinase may be at least about 2-fold, also 5-fold, also 10-fold, also 20-fold, also 50-fold, or at least about 100-fold less than the 1050 for any of the other kinases as determined in a generally accepted kinase activity assay.

Those of skill in the art given the benefit of the instant disclosure will appreciate that a presently disclosed RAF inhibitor can be used for the treatment or prevention of a disease or condition that is mediated by the RAF kinase to which the RAF inhibitor selectively inhibits. For instance, a B-RAF inhibitor disclosed herein is potentially useful for the treatment or prevention of a disease or condition medicated by B-RAF or a mutation thereof. Melanoma is an exemplary disease or condition medicated by B-RAF. Accordingly, a B-RAF inhibitor, such as ponatinib, is potentially useful for the treatment of melanoma.

In certain embodiments of any of the methods and compositions disclosed herein, the presently disclosed compound of Formula I can exhibit pan-inhibition against a particular RAF kinase (such as B-RAF). That is, the compound can exhibit activity against the wild-type target RAF kinase and all known mutations of that RAF-kinase. In alternative embodiments, the presently disclosed compounds of Formula I can exhibit pan-inhibition against all RAF-kinases (wild type) and all known mutations of all RAF-kinases.

In another aspect, this disclosure provides methods of treating a RAF kinase mediated disease or condition by administering to the subject an effective amount of a composition including a compound of Formula I in combination with one or more other therapies or medical procedures effective in treating the cancer. Other therapies or medical procedures include suitable anticancer therapy (e.g. drug therapy, vaccine therapy, gene therapy, photodynamic therapy) or medical procedure (e.g. surgery, radiation treatment, hyperthermia heating, bone marrow or stem cell transplant). In one aspect, the one or more suitable anticancer therapies or medical procedures is selected from treatment with another tyrosine kinase inhibitor (e.g., vemurafenib), a chemotherapeutic agent (e.g. chemotherapeutic drug such as decarbazine), radiation treatment (e.g. x-ray, γ-ray, or electron, proton, neutron, or a particle beam), hyperthermia heating (e.g. microwave, ultrasound, radiofrequency ablation), Vaccine therapy (e.g. AFP gene hepatocellular carcinoma vaccine, AFP adenoviral vector vaccine, AG-858, allogeneic GM-CSF-secretion breast cancer vaccine, dendritic cell peptide vaccines), gene therapy (e.g. Ad5CMV-p53 vector, adenovector encoding MDA7, adenovirus 5-tumor necrosis factor alpha), photodynamic therapy (e.g. aminolevulinic acid, motexafin lutetium), surgery, and bone marrow and stem cell transplantation.

### Therapy

Therapy according to the invention may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the patient's disease, and how the patient responds to the treatment. Additionally, a person having a greater risk of developing a RAF kinase mediated disease or condition (e.g., a person who is genetically predisposed) may receive ponatinib therapy to inhibit or delay symptoms of the disease.

Methods of diagnosing patients as having or being at risk of having a RAF kinase mediated disease or condition are well-known in the art. Review of a patient's symptoms, activity, medications, concurrent medical problems, or possible toxic exposures can be useful in making a RAF kinase mediated disease diagnosis. In addition, a patient may be tested for the presence or absence of genetic mutations that can indicate an increased likelihood of having a RAF kinase mediated disease. For example, the presence of one or more specific mutations or polymorphisms in the B-RAF gene such as V600E may be used to diagnose a patient as having or being at risk of having melanoma. See, e.g., package insert for ZELBORAF, which is hereby incorporated by reference.

### Compounds of Formula I

As discussed herein, certain Abl inhibitors have been found to be suitable candidates for their ability to inhibit RAF and thus treat or prevent a RAF kinase mediated disease or condition. One class of such inhibitors includes the compounds disclosed in WO 2007/075869.

RAF inhibitors suitable for the presently disclosed methods and pharmaceutical compositions are compounds of Formula I:

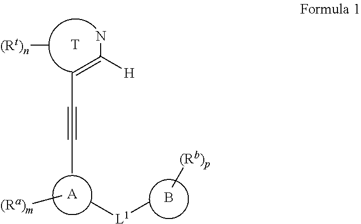

or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;

or a pharmaceutically acceptable salt, solvate or hydrate thereof.

The following portions of this section disclose various subgenuses of compounds of Formula I. In each subgenus, any variable not explicitly mentioned has the meaning defined by the genus immediately above, unless explicitly indicated otherwise.

In certain embodiments in the compound of Formula I, Ring T is:

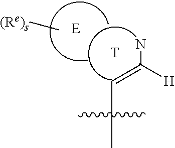

wherein Ring E is a 5- or 6-membered unsaturated ring comprising 0-3 heteroatoms selected from O, N, and S, and s is 0, 1, 2, 3 or 4.

Compounds useful for methods and pharmaceutical compositions disclosed herein include those in which Ring T has the following structure:

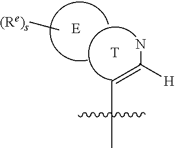

where Ring E is a 5- or 6-membered unsaturated ring (formed by two R^{t} groups together with the Ring T atoms to which they are attached, as described above) and s is 0, 1, 2, 3 or 4. These are illustrated by the compounds of Formula I in which the fused Ring T ring system is one of the following (in which one of the optional R^{e} substituents is depicted):

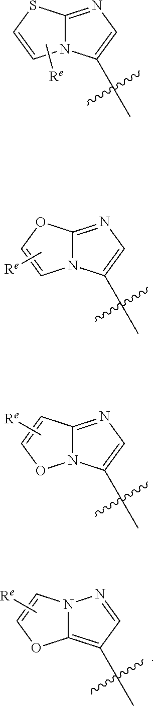

In certain embodiments in the compounds of Formula I, Ring T is a bicyclic heteroaryl ring selected from:

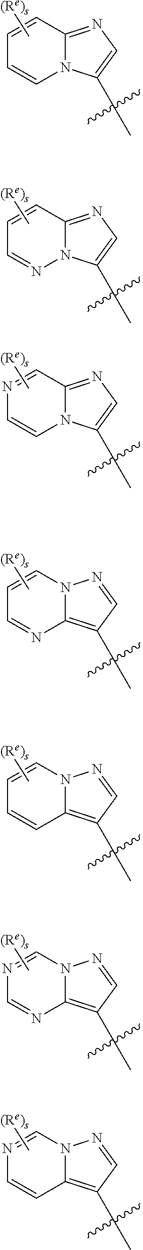

and s is 0, 1, 2, 3 or 4.

For the previously described class and subclasses of compounds, as in all compounds of this invention, Ring A and Ring B are as previously defined.

In certain of these embodiments, Ring A is selected from:

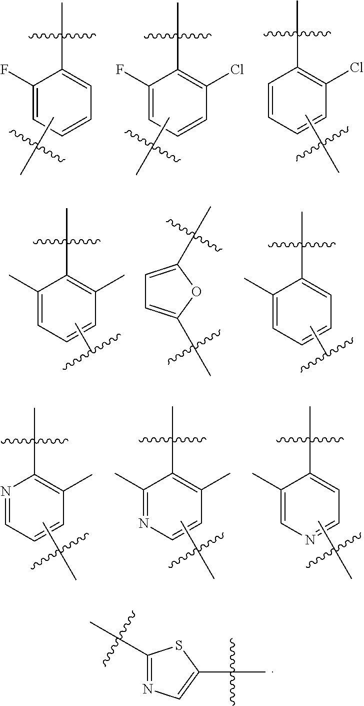

In certain embodiments in the compounds of Formula I, Ring B is a 5 or 6-membered aryl or heteroaryl ring as defined herein.

In certain of these embodiments. Ring B is:

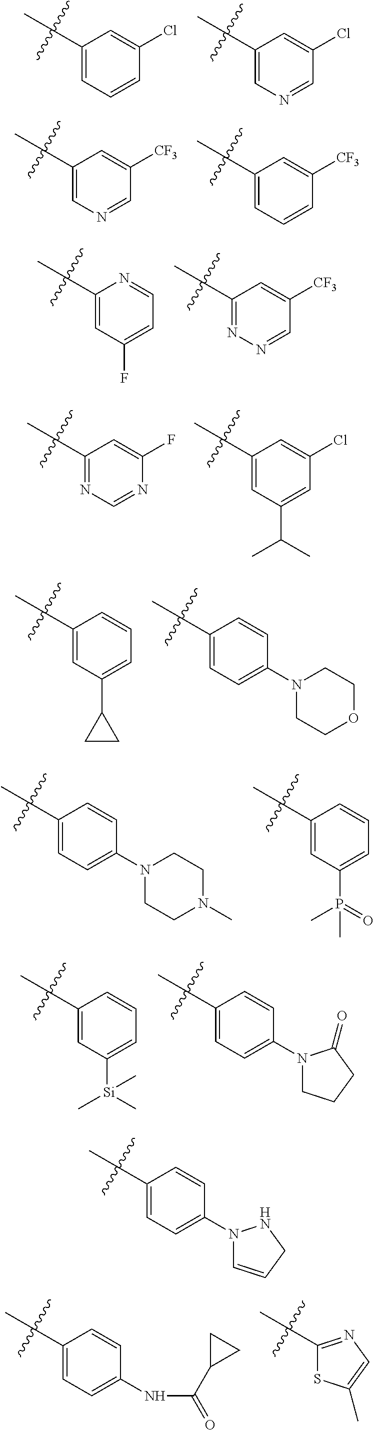

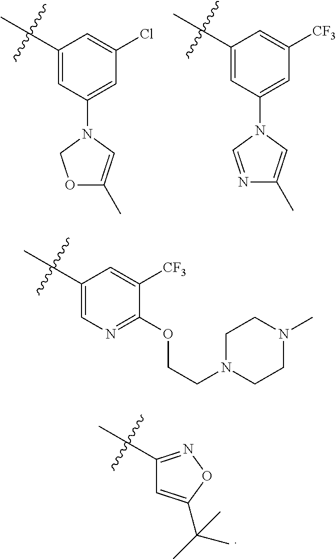

In certain embodiments in the compounds of Formula I, Rings A and B are aryl.

In certain embodiments in the compounds of Formula I, one of the R^{b} substituents is a 5- or 6-membered ring (Ring C), which may be heteroaryl or heterocyclic, comprising carbon atoms and 1-3 heteroatoms independently selected from O, N and S(O)ᵣ, and Ring C being optionally substituted on carbon or heteroatom(s) with 1 to 5 substituents R^{c}.

In certain embodiments, the RAF inhibitor is a compound of the Formula II:

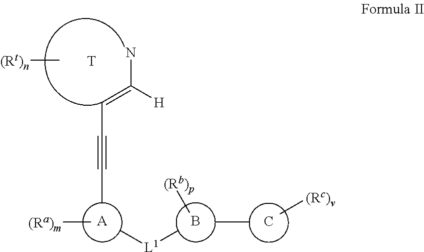

wherein:

Ring C is a 5- or 6-membered heterocyclic or heteroaryl ring, comprising carbon atoms and 1-3 heteroatoms independently selected from O, N and S(O)ᵣ;

R^{c}, at each occurrence, is independently selected from halo, ═O, —CN, —NO₂, —R⁴, —OR², —NR²R³, —C(O)YR², —OC(O)YR², —NR²C(O)YR², —Si(R²)₃, —SC(O)YR², —NR²C(═S)YR², —OC(═S)YR², —C(═S)YR², —YC(═NR³)YR², —YP(═O)(YR⁴)(YR⁴), —NR²SO₂R², —S(O)ᵣR², —SO₂NR²R³ and —NR²SO₂NR²R³, wherein each Y is independently a bond, —O—, —S— or —NR³—; and,

v is 0, 1, 2, 3, 4 or 5.

In certain of these embodiments, Ring C is selected from the group consisting of:

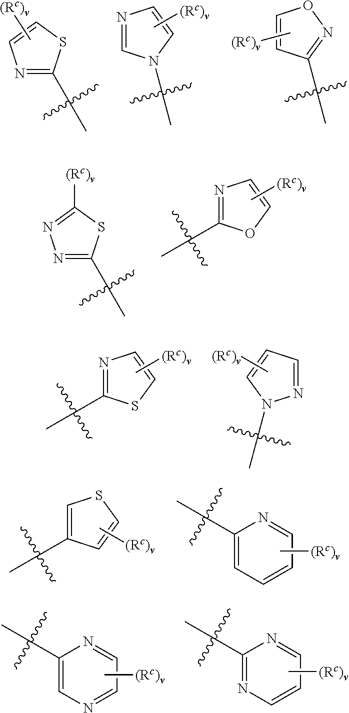

in which R^{c} and v are as defined above.

In certain embodiments in the compounds of Formula I where Ring C is present, Rings A and B are aryl.

In certain embodiments in the compound of Formula I where Ring C is present, Ring T is:

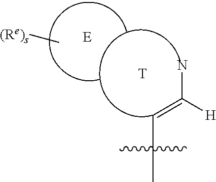

wherein Ring E is a 5- or 6-membered unsaturated ring comprising 0-3 heteroatoms selected from O, N, and S, and s is 0, 1, 2, 3 or 4.

Illustrative subsets of such compounds of Formula I include those having the following structures:

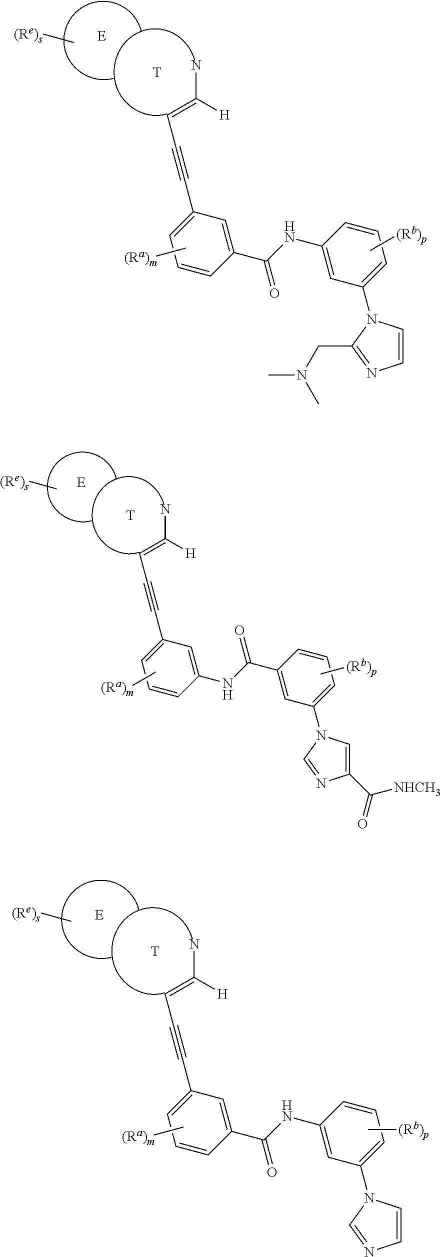

as embodied by the following non-limiting illustrative examples:

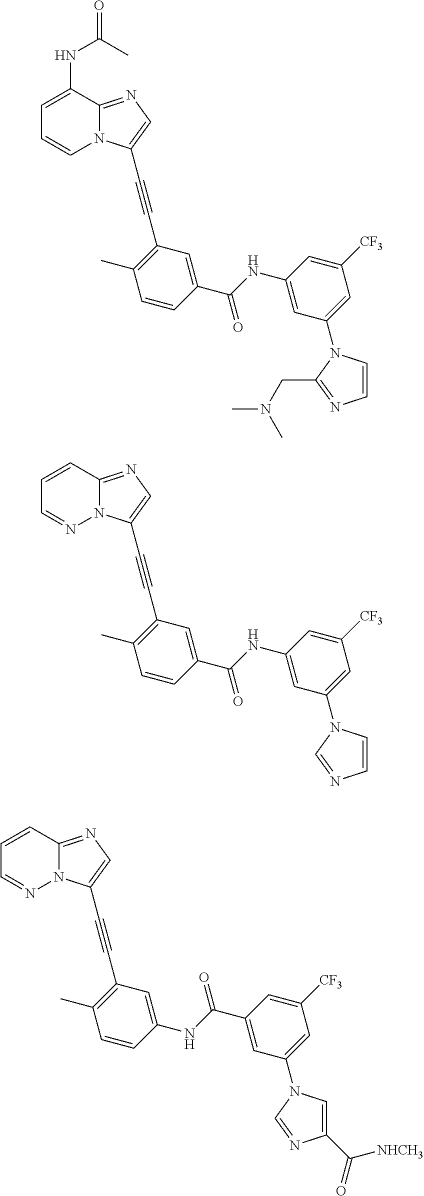 [SMILES: CC(=O)Nc1cccn2c(C#Cc3cc(C(=O)Nc4cc(-n5ccnc5CN(C)C)cc(C(F)(F)F)c4)ccc3C)cnc12 ; Cc1ccc(C(=O)Nc2cc(-n3ccnc3)cc(C(F)(F)F)c2)cc1C#Cc1cnc2cccnn12 ; CNC(=O)c1cn(-c2cc(C(=O)Nc3ccc(C)c(C#Cc4cnc5cccnn45)c3)cc(C(F)(F)F)c2)cn1]

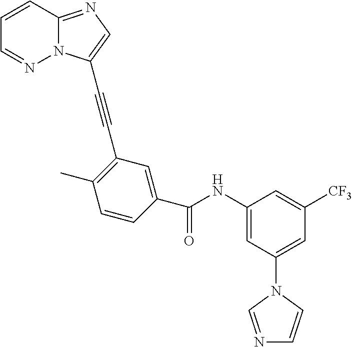 [SMILES: Cc1ccc(C(=O)Nc2cc(-n3ccnc3)cc(C(F)(F)F)c2)cc1C#Cc1cnc2cccnn12]

in which several illustrative -[Ring A]-[L¹]-[Ring B]-[Ring C]- portions are depicted.

In certain embodiments in the compounds of Formula I, Ring C is imidazolyl. Compounds of interest include among others, compounds of Formula II in which Ring C is an imidazole ring, optionally substituted with one or more R^{c} groups. Of particular interest, are compounds of this subclass in which Ring C bears a single lower alkyl (e.g., methyl) R^{c} group.

In certain of these embodiments where Ring C is imidazolyl, the RAF inhibitor is a compound selected from Formulae IIa, IIb, or IIc:

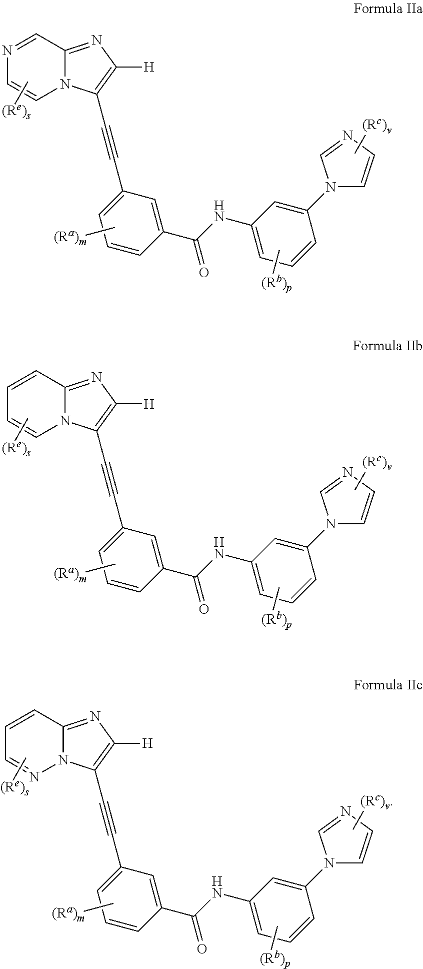

In certain embodiments within these embodiments, s is 0; m, p and v are 1; R^{a} and R^{c} are methyl; and R^{b} is CF₃.

In certain embodiments in the compounds of Formula I, the RAF inhibitor is a compound of the formula:

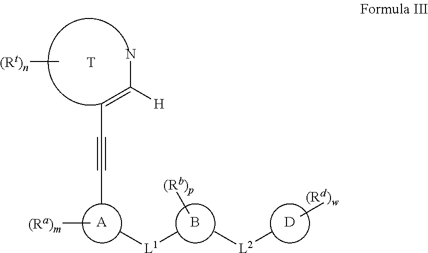

wherein:

Ring D represents a 5-, 6-heterocyclic or heteroaryl ring comprising carbon atoms and 1-3 heteroatoms independently selected from O, N and S(O)ᵣ;

L² is (CH₂)_{z}, O(CH₂)ₓ, NR³(CH₂)ₓ, S(CH₂)ₓ or (CH₂)ₓNR³C(O)(CH₂)ₓ in either direction;

R^{d}, at each occurrence, is selected from the group consisting of H, halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;

R¹, R² and R³ are independently selected from H, alkyl, alkenyl, alkynyl, cycloalkyl, cycloalkenyl, cycloalkynyl, aryl, heterocyclic and heteroaryl;

alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;

each occurrence of R⁴ is independently selected from alkyl, alkenyl, alkynyl, cycloalkyl, cycloalkenyl, cycloalkynyl, aryl, heterocyclic and heteroaryl;

each of the alkyl, alkenyl, alkynyl, cycloalkyl, cycloalkenyl, cycloalkynyl, aryl, heterocyclic and heteroaryl moieties is optionally substituted;

p is 0, 1, 2, 3 or 4;

w is 0, 1, 2, 3, 4 or 5;

x is 0, 1, 2 or 3; and,

z is 1, 2, 3 or 4.

In certain of these embodiments where Ring D is present, Ring T has the following structure:

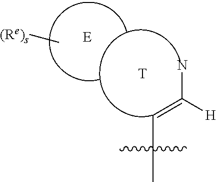

wherein Ring E is a 5- or 6-membered unsaturated ring comprising 0-3 heteroatoms selected from O, N, and S, and s is 0, 1, 2, 3 or 4.

Non-limiting examples of such compounds include those having the following structures:

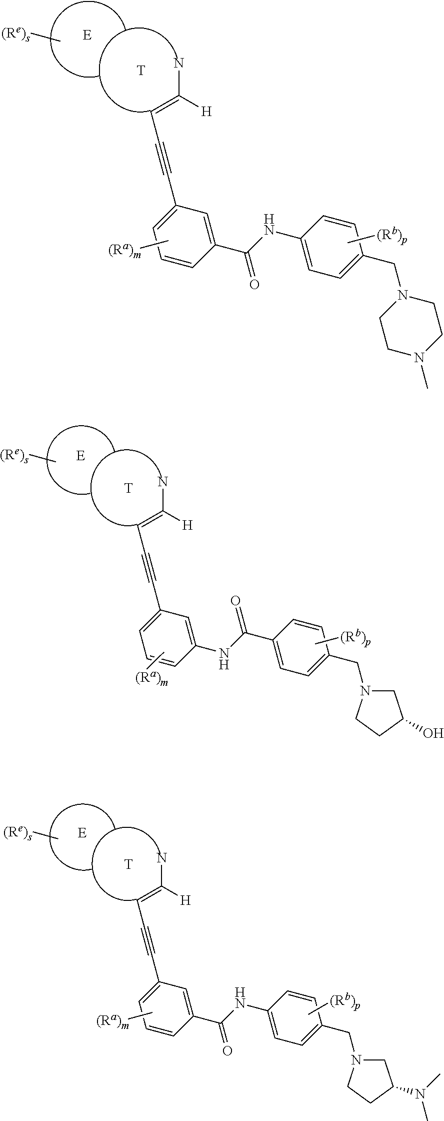

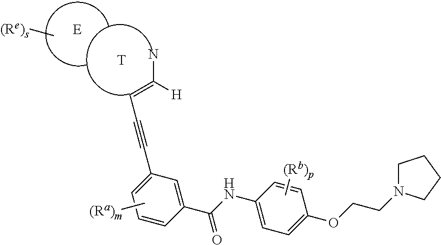

as illustrated by the following examples:

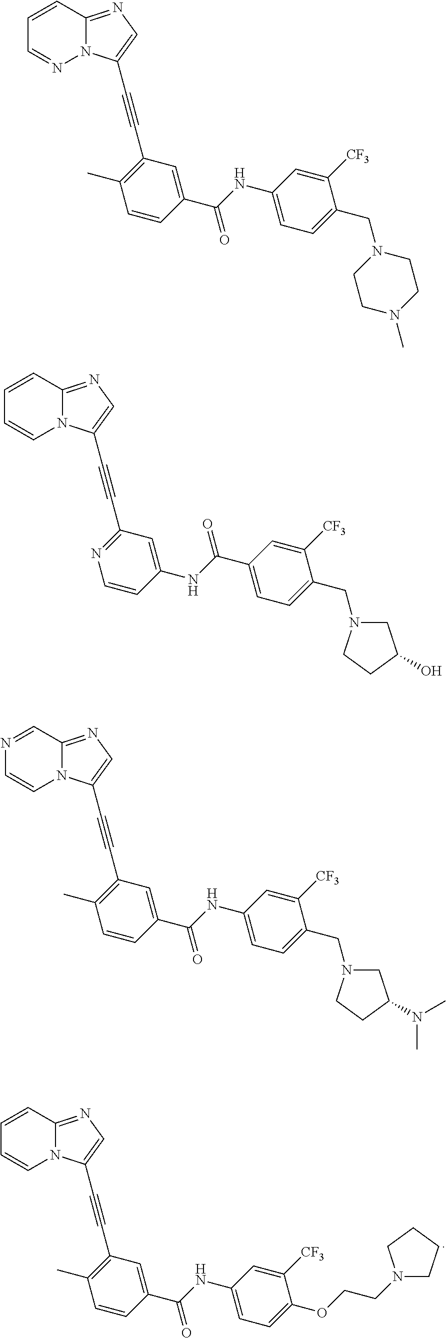 [SMILES: Cc1ccc(C(=O)Nc2ccc(CN3CCN(C)CC3)c(C(F)(F)F)c2)cc1C#Cc1cnc2cccnn12 ; O=C(Nc1ccnc(C#Cc2cnc3ccccn23)c1)c1ccc(CN2CC[C@@H](O)C2)c(C(F)(F)F)c1 ; Cc1ccc(C(=O)Nc2ccc(CN3CC[C@@H](N(C)C)C3)c(C(F)(F)F)c2)cc1C#Cc1cnc2cnccn12 ; Cc1ccc(C(=O)Nc2ccc(OCCN3CCCC3)c(C(F)(F)F)c2)cc1C#Cc1cnc2ccccn12]

In certain of these embodiments where Ring D is present, Rings A and B are aryl.

In certain of these embodiments where Ring D is present, Ring T is a bicyclic heteroaryl ring selected from:

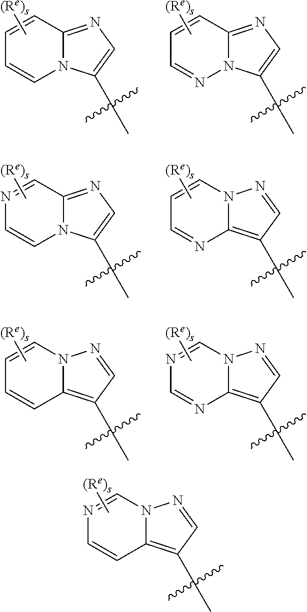

and s is 0, 1, 2, 3 or 4.

Non-limiting, illustrative examples of -[Ring B]-[L²]-[Ring D] moieties in compounds of Formula III include among others:

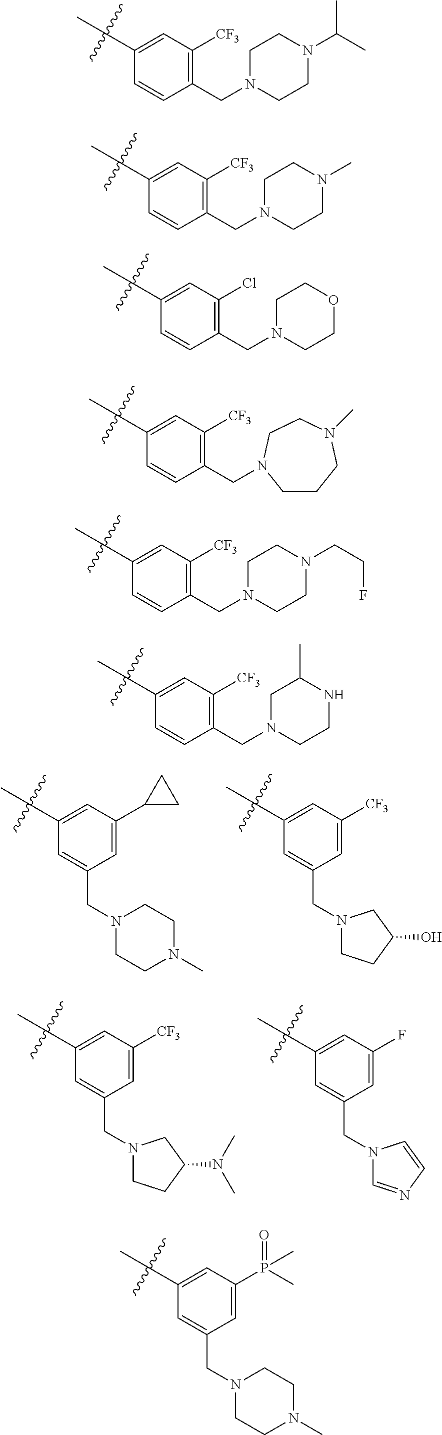

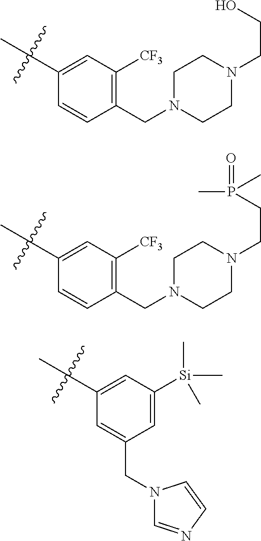

In certain embodiments in the compounds of Formula I, compounds of interest include among others, compounds of Formula III in which Ring D is a piperazine ring, substituted on nitrogen with R^{d}. Of particular current interest, are compounds of this subclass in which R^{d} is a substituted or unsubstituted lower (i.e., 1-6 carbon) alkyl as illustrated by N-methylpiperazine moieties in some of the following examples.

In certain of these embodiments where Ring D is present, Ring D is piperazinyl and L² is CH₂. In certain of these embodiments, the RAF inhibitor is a compound selected from Formulae IIIa, IIIb, and IIIc:

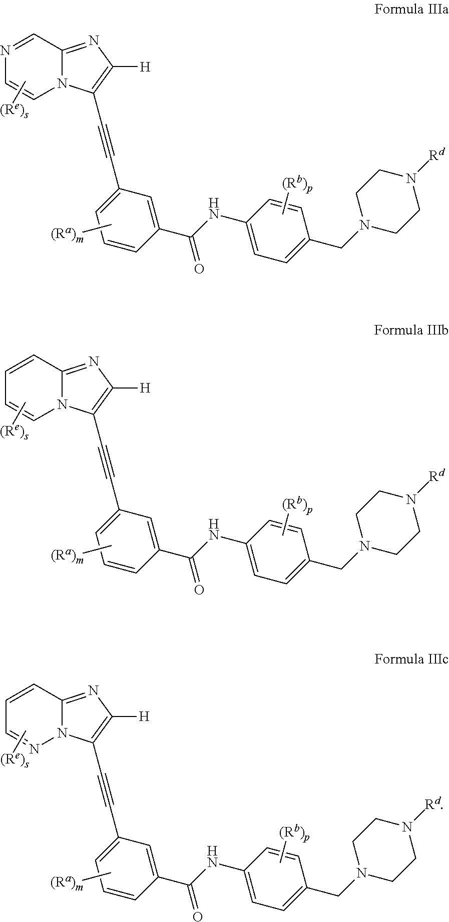

In certain embodiments within these embodiments, s is 0, m is 1, p is 1, R^{e} is methyl, R^{b} is CF₃, and R^{d} is methyl or —CH₂CH₂OH.

In certain embodiments in the compounds of Formula II and Formula III, Ring T is any 6/5 fused heteroaryl ring system, optionally substituted with up to three R^{e} groups. Of particular interest are compounds in which s is 0. Also of interest are those in which s is 1-3 and at least one R^{e} is halo, lower alkyl, alkoxy, amino, —NH-alkyl, —C(O)NH-alkyl, —NHC(O)-alkyl, —NHC(O)NH-alkyl, —NHC(NH)-alkyl, —NHC(NH)NH₂, —NH(CH₂)ₓ-heteroaryl, —NH(CH₂)ₓ-heterocycle, —NH(CH₂)ₓ-aryl or —(CH₂)ₓC(O)NH₂, in which x is 0, 1, 2 or 3 and “alkyl” includes straight (i.e., unbranched and acyclic), branched and cyclic alkyl groups and in which aryl, heteroaryl, heterocyclyl rings are optionally substituted. Illustrative, non-limiting, examples of the foregoing include compounds of Formulas II and III in which Ring T is one of the following:

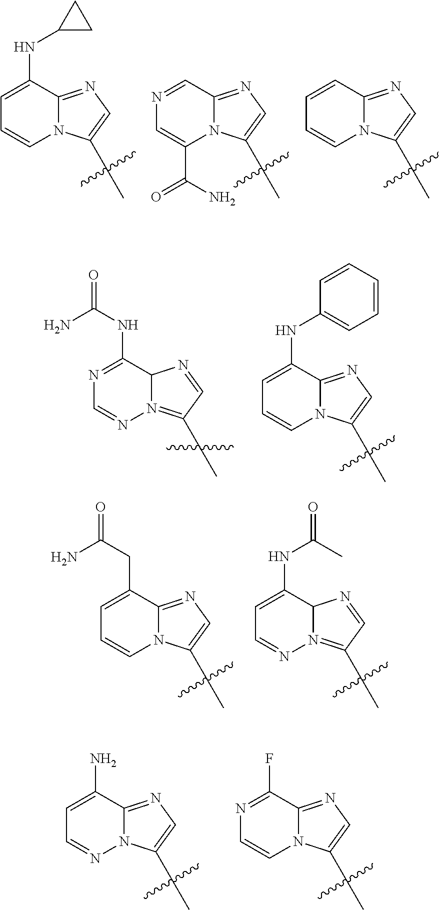

In certain embodiments in the compounds of Formula II and Formula III, Ring T is an optionally substituted imidazo[1,2-a]pyridine, imidazo[1,2-b]pyridazine, imidazo[1,2-a]pyrazine, pyrazolo[1,5-a]pyrimidine, pyrazolo[1,5-a]pyridine, pyrazolo[1,5-c]pyrimidine, and pyrazolo[1,5-a][1,3,5]triazine.

In certain of these embodiments in the compounds of Formula II and Formula III, Rings A and B are aryl.

Illustrative, non-limiting examples of this subclass include compounds of Formulas IIa, IIb, IIe, IIIa, IIIb and IIIc:

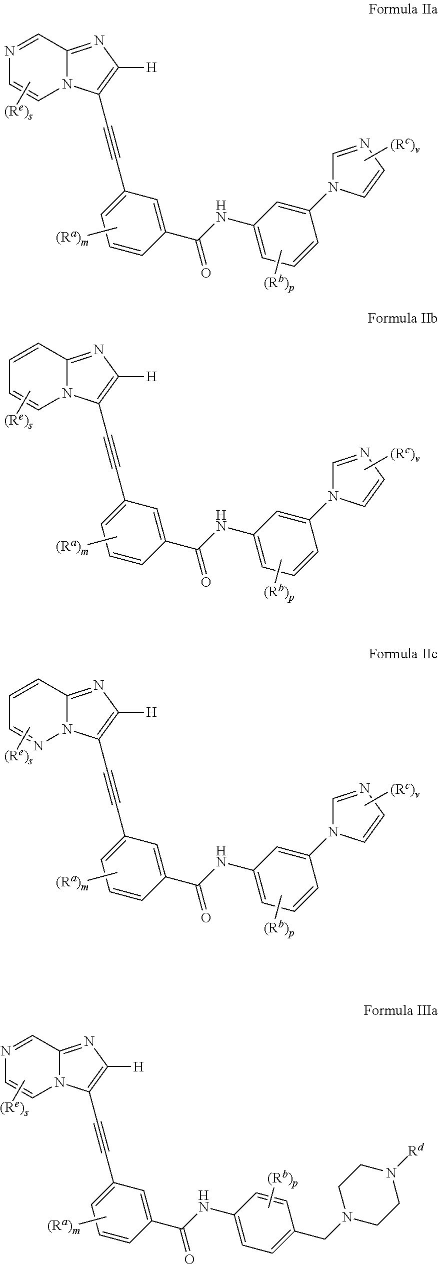

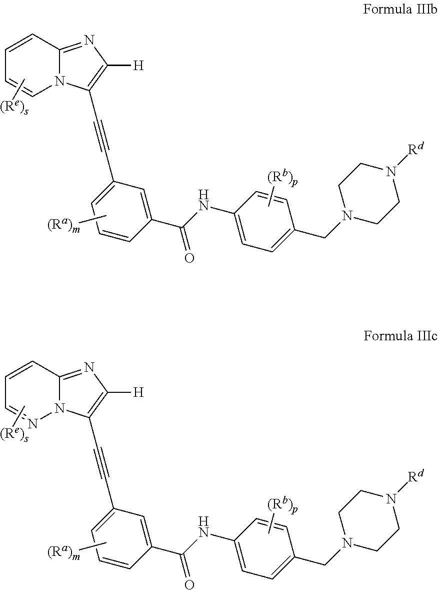

in which the variables, e.g., R^{a}, R^{b}, R^{c}, R^{d}, R^{e}, m and p, are as previously defined and s is an integer from 0 to 4.

In certain embodiments in the compounds of Formulas IIa, IIb and IIc, s is 0; m, p and v are 1; and, R^{a} is CH₃, R^{b} is CF₃ and R^{e} is methyl.

In certain embodiments in the compounds of Formulas Ma, IIIb, IIIc, s is 0; m and p are 1; and, R^{a} is CH₃, R^{b} is CF₃ and R^{d} is CH₃ or CH₂CH₂OH.

In certain embodiments, the RAF inhibitor is a compound selected from the group consisting of:
N-(3-(1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyrazin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyridin-3-ylethynyl)-4-methyl-N-(3-(4-methyl-1H-imidazol-1-yl)-5-(trifluoromethyl)phenyl)benzamide;
N-(3-(1H-imidazol-1-yl)-5-(trifluoromethyl)phenyl)-3-(imidazo[1,2-a]pyridin-3-ylethynyl)-4-methylbenzamide;
3-(Imidazo[1,2-a]pyridin-3-ylethynyl)-4-methyl-N-(4-(trifluoromethyl)pyridin-2-yl)benzamide;
N-(5-tert-butylisoxazol-3-yl)-3-(imidazo[1,2-a]pyridin-3-ylethynyl)-4-methylbenzamide;
3-(Imidazo[1,2-a]pyridin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide;
3-((8-Acetamidoimidazo[1,2-a]pyridin-3-yl)ethynyl)-4-methyl-N-(4-(trifluoromethyl)pyridin-2-yl)benzamide;
N-(3-(1H-imidazol-1-yl)-5-(trifluoromethyl)phenyl)-3-((8-acetamidoimidazo[1,2-a]pyridin-3-yl)ethynyl)-4-methylbenzamide;
4-Methyl-3-((8-(4-(methylsulfonyl)phenylamino)imidazo[1,2-a]pyridin-3-yl)ethynyl)-N-(4-(trifluoromethyl)pyridin-2-yl)benzamide;
4-methyl-3-((8-(4-sulfamoylphenylamino)imidazo[1,2-a]pyridin-3-yl)ethynyl)-N-(4-(trifluoromethyl)pyridin-2-yl)benzamide;
(R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide;
N-(3-(Imidazo[1,2-b]pyridazin-3-ylethynyl)-4-methylphenyl)-4-((4-methylpiperazin-1-yl)methyl)-3-(trifluoromethyl)benzamide;
3-(Imidazo[1,2-b]pyridazin-3-ylethynyl)-4-methyl-N-(4-((4-methylpiperazin-1-yl)methyl)-3-(trifluoromethyl)phenyl)benzamide;
N-(3-Chloro-4-((4-methylpiperazin-1-yl)methyl)phenyl)-3-(imidazo[1,2-b]pyridazin-3-ylethynyl)-4-methylbenzamide;
N-(3-Cyclopropyl-4-((4-methylpiperazin-1-yl)methyl)phenyl)-3-(imidazo[1,2-b]pyridazin-3-ylethynyl)-4-methylbenzamide;
3-(Imidazo[1,2-b]pyridazin-3-ylethynyl)-N-(4-((4-methylpiperazin-1-yl)methyl)-3-(trifluoromethyl)phenyl)benzamide;
N-(4-((4-(2-Hydroxyethyl)piperazin-1-yl)methyl)-3-(trifluoromethyl)phenyl)-3-(imidazo[1,2-b]pyridazin-3-ylethynyl)-4-methylbenzamide; and
3-(Imidazo[1,2-b]pyridazin-3-ylethynyl)-4-methyl-N-(4-(piperazin-1-ylmethyl)-3-(trifluoromethyl)phenyl)benzamide,
   or a pharmaceutically acceptable salt thereof.

A RAF inhibitor of particular interest that is useful for the presently disclosed methods and pharmaceutical compositions is 3-(Imidazo[1,2-b]pyridazin-3-ylethynyl)-4-methyl-N-(4-((4-methylpiperazin-1-yl)methyl)-3-(trifluoromethyl)phenyl)benzamide or a pharmaceutically acceptable salt thereof. A pharmaceutically acceptable salt of particular interest for this compound (ponatinib) is its hydrochloride salt.

In certain embodiments in the compounds of Formula I, the RAF inhibitor is a compound of the formula:

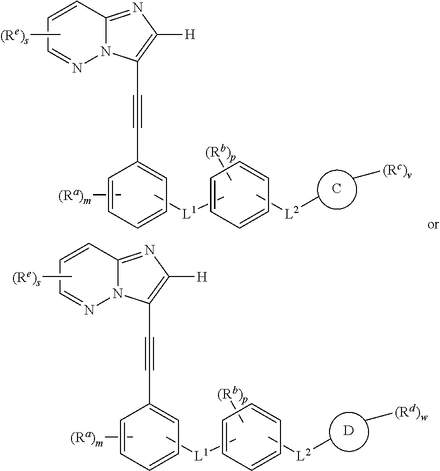

wherein:

L¹ is NR¹C(O) or C(O)NR¹;

Ring D is a 5- or 6-membered heterocyclyl or heteroaryl ring comprising carbon atoms and 1-3 heteroatoms independently selected from O, N, and S(O)ᵣ;

Ring C is a 5- or 6-membered heterocyclyl or heteroaryl ring, comprising carbon atoms and 1-3 heteroatoms independently selected from O, N, and S(O)ᵣ;

L² is —(CH₂)_{z}—;

each occurrence of R^{a} is independently selected from the group consisting of halo, alkyl, and cycloalkyl;

each occurrence of R^{b} is independently selected from the group consisting of halo, alkyl, and cycloalkyl;

each occurrence of R^{e} is independently selected from the group consisting of halo, alkyl, and cycloalkyl;

each occurrence of R^{d} is independently selected from the group consisting of halo, alkyl, cycloalkyl, and —NR²R³;

each occurrence of R^{e} is independently selected from the group consisting of halo, alkyl, cycloalkyl, —NR²R³, alkoxy, amino, —NH-alkyl, —C(O)NH-alkyl, —NHC(O)-alkyl, —NHC(O)NH-alkyl, —NHC(NH)-alkyl, —NHC(NH)NH₂, —NH(CH₂)ₓ-heteroaryl, —NH(CH₂)ₓ-heterocyclyl, —NH(CH₂)ₓ-aryl, and —(CH₂)ₓC(O)NH₂, wherein x is 0, 1, 2 or 3;

each of R¹, R² and R³ is independently selected from H, alkyl, alkenyl, alkynyl, cycloalkyl, cycloalkenyl, cycloalkynyl, aryl, heterocyclyl, and heteroaryl, or R² and R³, taken together with the nitrogen atom to which at least one of R² and R³ is attached, form a 5- or 6-membered heterocyclyl or heteroaryl ring;

each of the alkyl, alkenyl, alkynyl, cycloalkyl, cycloalkenyl, cycloalkynyl, and heterocyclyl moieties is unsubstituted or substituted with one or more groups selected from amino, alkylamino, dialkylamino, aminocarbonyl, halogen, alkyl, alkenyl, alkynyl, aryl, heteroaryl, carbocyclyl, heterocyclyl, alkylaminocarbonyl, dialkylaminocarbonyl, alkylaminocarbonyloxy, dialkylaminocarbonyloxy, nitro, cyano, carboxy, alkoxycarbonyl, alkylcarbonyl, hydroxy, alkoxy, acyloxy, haloalkoxy, ═O, ═S, ═NH, ═NNR²R³, ═NNHC(O)R², ═NNHCO₂R², and ═NNHSO₂R², and each of the aryl and heteroaryl moieties is unsubstituted or substituted with one or more groups selected from amino, alkylamino, dialkylamino, aminocarbonyl, halogen, alkyl, alkenyl, alkynyl, aryl, heteroaryl, carbocyclyl, heterocyclyl, alkylaminocarbonyl, dialkylaminocarbonyl, alkylaminocarbonyloxy, dialkylaminocarbonyloxy, nitro, cyano, carboxy, alkoxycarbonyl, alkylcarbonyl, hydroxy, alkoxy, acyloxy, and haloalkoxy;

m is 0, 1, 2, 3, or 4;

p is 0, 1, 2, 3, or 4;

r is 0, 1, or 2;

s is 0, 1, 2, or 3;

v is 0, 1, 2, 3, 4, or 5;

w is 0, 1, 2, 3, 4, or 5; and

z is 1, 2, 3 or 4;

or a pharmaceutically acceptable salt thereof.

### Formulations, Dosage and Administration

Compounds of Formula I can be formulated into a pharmaceutical composition that comprises a compound of Formula I (as an active pharmaceutical ingredient) or a pharmaceutically acceptable salt thereof and a pharmaceutically acceptable carrier. Similarly, ponatinib, or a pharmaceutically acceptable salt thereof, such as the mono HCl salt, can be formulated for administration, such as oral administration, using any of the materials and methods useful for such purposes.

Pharmaceutically acceptable compositions containing a compound of Formula I suitable for administration may be formulated using conventional materials and methods, a wide variety of which are well known. While the composition may be in solution, suspension or emulsion form, solid oral dosage forms such as capsules, tablets, gel caps, caplets, etc. are of particular interest. Methods well known in the art for making formulations, including the foregoing unit dosage forms, are found, for example, in “Remington: The Science and Practice of Pharmacy” (20th ed., ed. A. R. Gennaro, 2000, Lippincott Williams & Wilkins). A compound of Formula I such as ponatinib (or a pharmaceutically acceptable salt thereof) may be provided neat in capsules, or combined with one or more optional, pharmaceutically acceptable excipients such as fillers, binders, stabilizers, preservatives, glidants, disintegrants, colorants, film coating, etc., as illustrated below.

For example, white opaque capsules were prepared containing nominally 2 mg of ponatinib free base, provided as the hydrochloride salt, with no excipients. White opaque capsules were also prepared containing 5 mg, 15 mg, or 20 mg of ponatinib free base, provided as the hydrochloride salt, mixed with conventional excipients. Inactive ingredients used as excipients in an illustrative capsule blend include one or more of a filler, a flow enhancer, a lubricant, and a disintegrant. For instance, a capsule blend was prepared for the 5, 15 and 20 mg capsules, containing the ponatinib HCl salt plus colloidal silicon dioxide (ca. 0.3% w/w, a flow enhancer), lactose anhydrous (ca. 44.6% w/w, a filler), magnesium stearate (ca. 0.5% w/w, a lubricant), microcrystalline cellulose (ca. 44.6% w/w, a filler), and sodium starch glycolate (ca. 5% w/w, a disintegrant). The capsule shell contains gelatin and titanium dioxide.

The formulation process used conventional blending and encapsulation processes and machinery. The hydrochloride salt of ponatinib and all blend excipients except magnesium stearate were mixed in a V-blender and milled through a screening mill. Magnesium stearate was added and the material was mixed again. The V-blender was sampled to determine blend uniformity. The blend was tested for bulk density, tap density, flow, and particle size distribution. The blend was then encapsulated into size “3”, size “4”, or size “1” capsule shells, depending upon the strength of the unit dosage form.

Ponatinib was also formulated into tablets using conventional pharmaceutical excipients, including one or more of a filler or a mixture of fillers, a disintegrant, a glidant, a lubricant, a film coating, and a coating solvent in a blend similar to that used in the higher strength capsules. For example, tablets may be prepared using the following relative amounts and proportions (weight/weight): ponatinib (90 g provided as the HCl salt, 15.0% w/w), colloidal silicon dioxide (1.2 g, 0.2% w/w), lactose monohydrate (240.9 g, 40.15% w/w), magnesium stearate (3 g, 0.5% w/w), microcrystalline cellulose (240.9 g, 40.15% w/w), and sodium starch glycolate (24 g, 4.0% w/w), with the amount of lactose monohydrate adjusted based on the amount of drug used.

Ponatinib and the excipients may be mixed using the same sort of machinery and operations as was used in the case of capsules. The resultant, uniform blend may then be compressed into tablets by conventional means, such as a rotary tablet press adjusted for target tablet weight, e.g. 300 mg for 45 mg tablets or 100 mg for 15 mg tablets; average hardness of e.g., 13 kp for 45 mg tablets and 3 kp for 15 mg tablets; and friability no more than 1%. The tablet cores so produced may be sprayed with a conventional film coating material, e.g., an aqueous suspension of Opadry® 11 White, yielding for example a ˜2.5% weight gain relative to the tablet core weight.

After formulation with an appropriate pharmaceutically acceptable carrier in a desired dosage, the compositions of disclosed herein can be administered to humans and other animals orally, rectally, parenterally, intracisternally, intravaginally, intraperitoneally, topically (as by transdermal patch, powders, ointments, or drops), sublingually, bucally, as an oral or nasal spray, or the like.

In accordance with the methods, kits, and pharmaceutical compositions of the invention, a treatment will typically consist of a plurality of doses of a compound of Formula I that is administered over a period of time. Administration may be one or multiple times daily, weekly (or at some other multiple day interval) or on an intermittent schedule, with that cycle repeated a given number of times (e.g., 2-10 cycles) or indefinitely.

Optimal dosing will depend in part on the route of administration. Effective doses may be calculated according to the body weight or body surface area. Optimization of the appropriate dosages can readily be made by one skilled in the art in light of pharmacokinetic data observed in human clinical trials. The final dosage regimen will be determined by the attending physician, considering various factors which modify the action of the drugs, e.g., the drug's specific activity, the severity of the damage and the responsiveness of the subject, the age, condition, body weight, sex and diet of the subject, and other clinical factors.

In certain embodiments, a compound of Formula I is administered at a unit dose of 5-80 mg (e.g., from 5 to 10 mg, 10 to 25 mg, 25 to 35 mg, 35 to 50 mg, 50 to 60 mg, or 60 to 80 mg). In certain of these embodiments, the unit dose is 5-45 mg or 15-45 mg. Preferred dosage strengths for ponatinib include, but are not limited to 15 mg, 30 mg, and 45 mg.

Oral administration is of particular interest in the practice of the various embodiments of this invention, including oral administration on a daily schedule or on an intermittent schedule as mentioned above and at the dose levels mentioned above. By way of non-limiting example, daily oral administration of 5-80 mg of ponatinib, and in some cases, 5-45 mg of ponatinib, are of particular current interest.

The amount and dosing schedule for ponatinib administered in any of the embodiments of the invention may be chosen or adjusted to produce a mean steady state trough concentration for ponatinib in plasma of from 5 to 200 nM (e.g., a mean steady state trough concentration for ponatinib of 5±2 nM, 8±3 nM, 12±3 nM, 15±3 nM, 20±5 nM, 30±5 nM, 40±5 nM, 50±10 nM, 60±10 nM, 80±20 nM, 100±20 nM, 120±20 nM, 150±25 nM, 175±25 nM, or 200±25 nM).

The amount and dosing schedule for ponatinib administered in any of the embodiments of the invention may be chosen or adjusted to be effective to measurably reduce RAF kinase activity in the subject.

In certain embodiments, the compound of Formula I is administered to the subject at an average daily dose of 3±1 mg, 5±2 mg, 8±2 mg, 12±3 mg, 15±3 mg, 20±4 mg, 25±5 mg, 30±6 mg, 40±8 mg, 45±9 mg, 50±10 mg, or 55±11 mg.

In certain embodiments, the compound of Formula I is administered to the subject on one or more days per week, including in some cases every day, every other day, every third day as well as schedules, such as, e.g., QDx6, QDx5 QDx4 QDx3 and QDx2 (i.e., 6, 5, 4, 3 or 2 days per week, respectively). On a given day, the drug may be given in one dose or may be divided into two or three doses administered during the course of the day (i.e., qd, bid or tid).

Because compounds of Formula I are orally bioavailable, a compound of Formula I such as ponatinib may be given orally as well as parenterally (e.g., i.v.) or by other pharmaceutically acceptable routes of administration. Thus, the active compounds of the disclosure may be formulated for oral, buccal, intranasal, parenteral (e.g., intravenous, intramuscular or subcutaneous), rectal administration, in a form suitable for administration by inhalation or insufflation, or the active compounds may be formulated for topical administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

For intranasal administration or administration by inhalation, the active compounds of the disclosure are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the disclosure and a suitable powder base such as lactose or starch.

The active compounds of the disclosure may be formulated for parenteral administration by injection, including using conventional catheterization techniques or infusion. Routes of parenteral administration also include intravenous, intramuscular and subcutaneous. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the disclosure may also be formulated in rectal compositions such as suppositories or retention enemas, e.g., containing conventional suppository bases such as cocoa butter or other glycerides.

For topical administration, a presently disclosed compound may be formulated as an ointment or cream.

Suitable modes of administration also include, but are not limited to, transdermal, vaginal, and ophthalmic.

### Synthesis of Compounds of Formula I

The synthesis of compounds of Formula I have been reported in WO 2007/075,869. For the convenience of the reader, the synthetic scheme is reproduced immediately below.

A compound of the present invention could be prepared as outlined in Scheme I to Scheme XIX and via standard methods known to those skilled in the art.

A palladium catalyzed Sonogashira coupling reaction is used to link the ‘top’ Ring T to the ‘bottom’ [Ring A]-[L¹]-[Ring B] moiety as illustrated in Scheme I and II. In Scheme I the Sonogashira coupling reaction is performed with an acetylenic ‘top’ Ring T and a ‘bottom’ [Ring A]-[L¹]-[Ring B] moiety which has been activated by the presence of a reactive group, W, which is an I, a Br or another reactive group permitting the desired coupling reaction. The variables in the W-[Ring A]-[L¹]-[Ring B] are as defined previously, Rings A and B being substituted with permitted R^{a} and R^{b} groups, respectively.

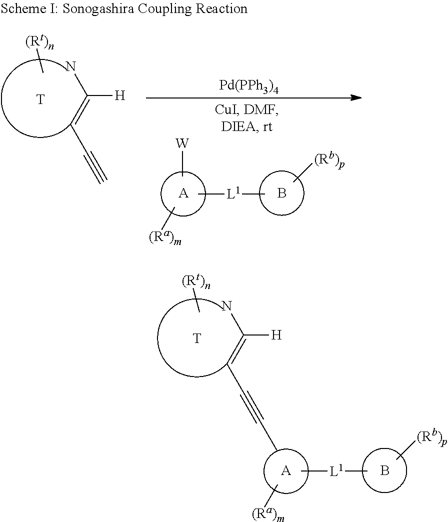

An alternative coupling reaction is described in Scheme II, in which Ring T is “activated” by the presence of a reactive group W (such as I or Br) and is coupled to the ‘bottom’ acetylenic [RingA]-L¹-[RingB] under similar Palladium catalyzed coupling conditions.

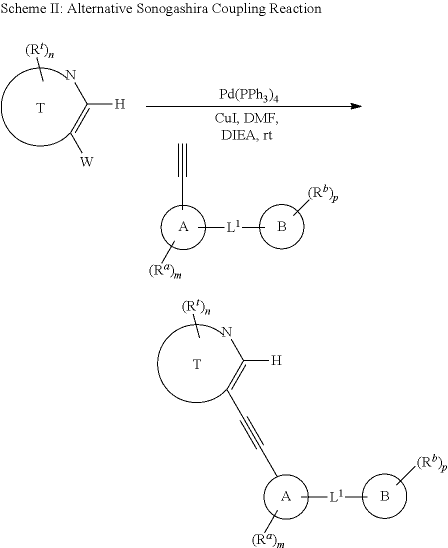

The Sonogashira coupling conditions described in Scheme I and II are applicable to all bicyclic heteroaryl Ring T's and useful to synthesize the compounds disclosed herein.

Several illustrative overall synthetic approaches to the preparation of the acetylenic Ring T moieties, based on known transformations, are illustrated below in Schemes III to VIII:

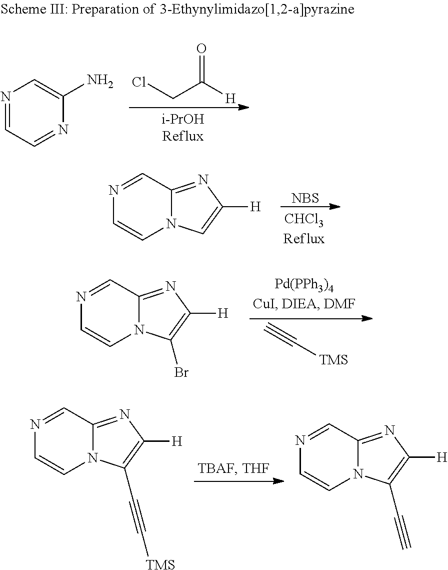

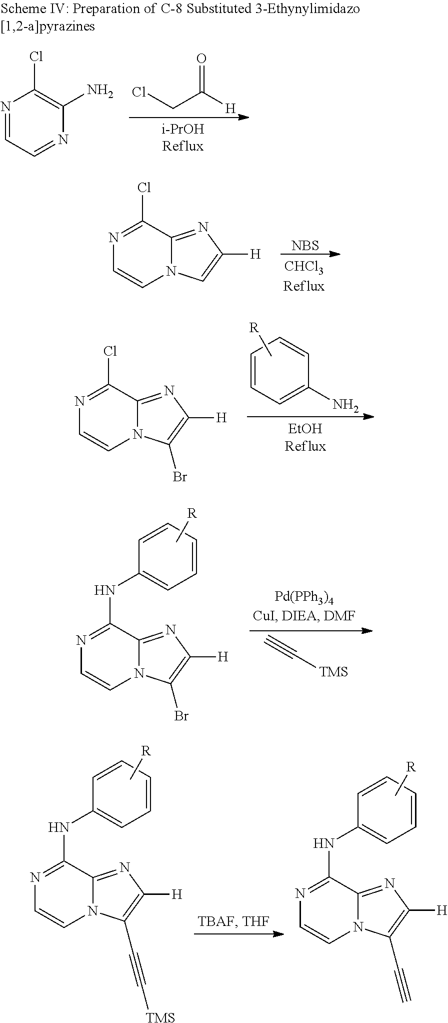

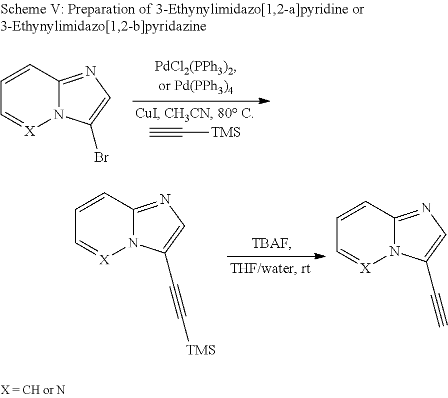

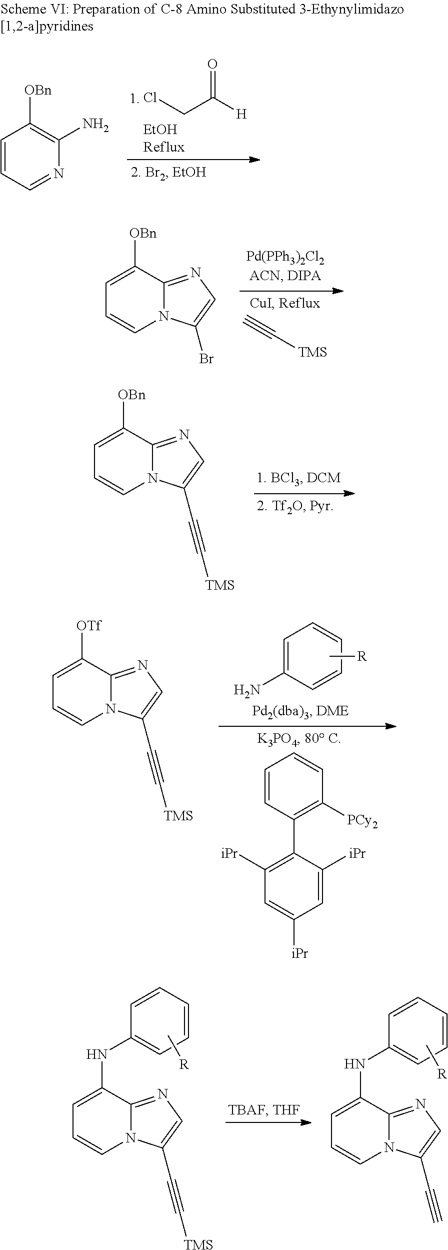

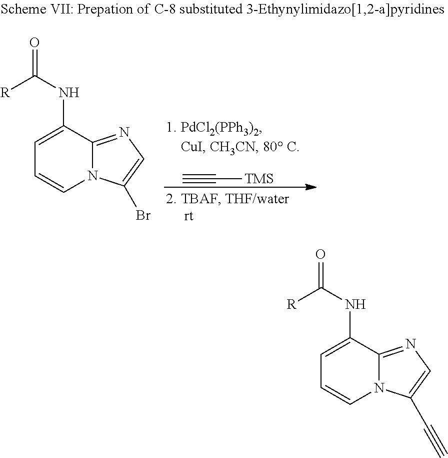

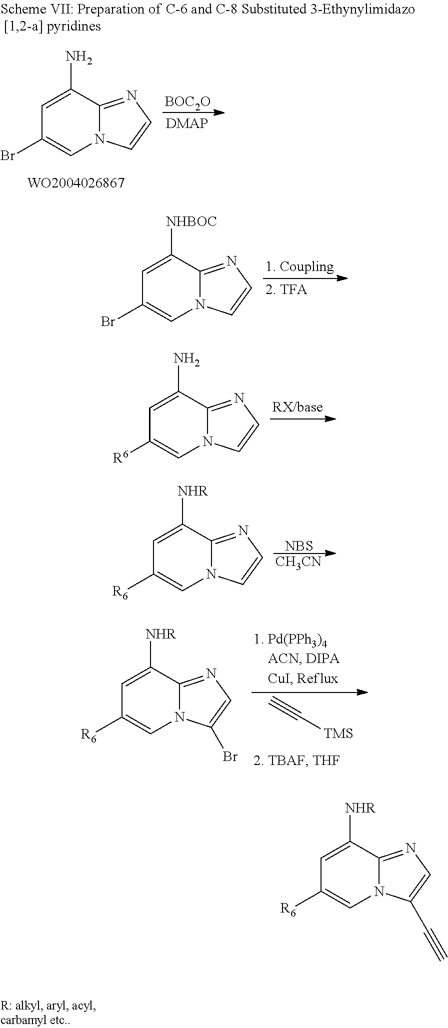

For the coupling step, see Malleron, J-L., Fiaud, J-C., Legros, J-Y. Handbook of Palladium Catalyzed Organic Reactions. San Diego: academic Press, 1997.

As one of ordinary skill in the art would recognize, these methods for the preparation of various substituted acetylenic Ring T groups, are widely applicable to various other fused bicyclic ring systems not shown.

Schemes IX to XIII below depict the synthesis of compounds of the formula W-[Ring A]-[L¹]-[Ring B] which are useful as intermediates in the coupling reaction described in Schemes I and II.

It should be apparent that intermediates of the formula:

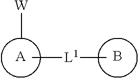

are of particular interest as their coupling reaction with the ‘top’ heteroaryl rings produces compounds of the present invention. The variable groups A, L¹ and B are as previously defined and are optionally substituted as described herein, and W is I or an alternative reactive group permitting the desired coupling reaction.

Illustrative such intermediates include among others those of those following structures:

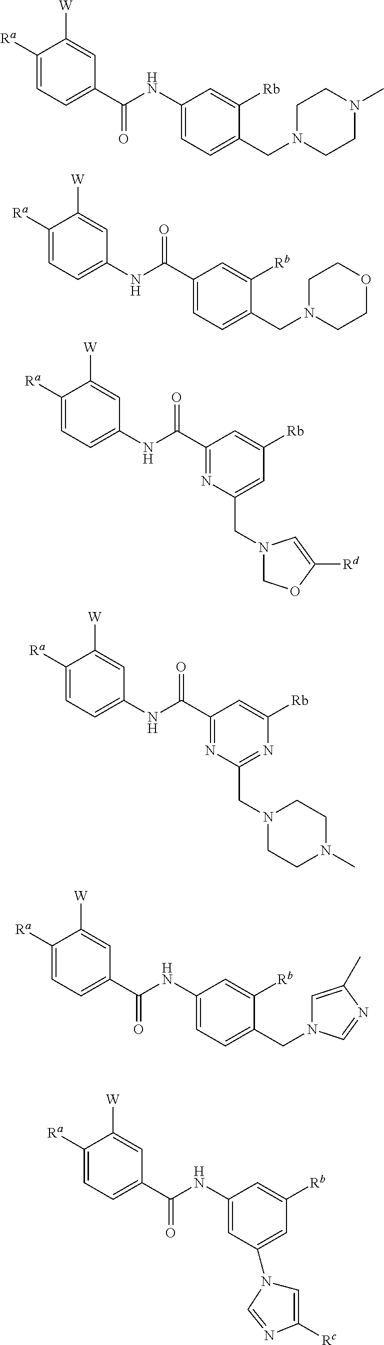

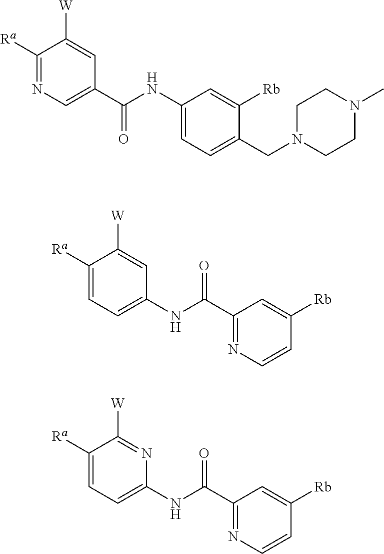

wherein the variables, e.g., R^{a}, R^{b}, R^{c} and R^{d}, are as previously defined. For instance, R^{a} in some embodiments is chosen from F or alkyl, e.g., Me, among others, and Rb in some embodiments is chosen from Cl, F, Me, t-butyl, —CF3 or —OCF3 among others. Those and other compounds of the formula W-[Ring A]-[L¹]-[Ring B] with the various permitted substituents are useful for preparing the corresponding compounds of the invention as are defined in the various formulae, classes and subclasses disclosed herein.

Some illustrative synthetic routes for the preparation of reagents and representative intermediates are presented below:

Scheme IX describes an illustrative synthesis of W-[Ring A]-[L¹]-[Ring B] in which Rings A and B are phenyl and L¹ is NHC(O).

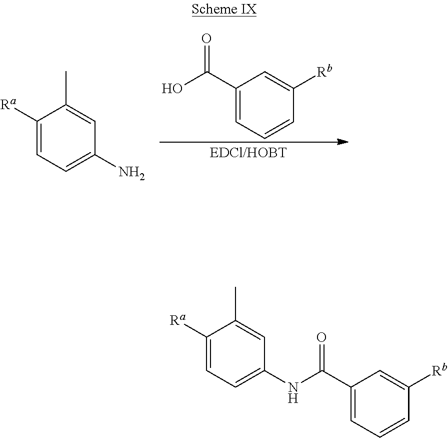

Scheme X depicts the synthesis of a variant of the foregoing in which Ring B is a 2-pyridine and L¹ is C(O)NH (i.e., in the other orientation).

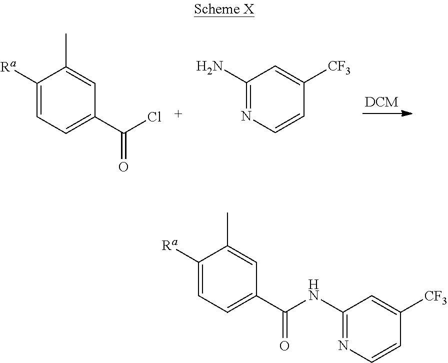

Schemes XI and XII, below, illustrate the synthesis of W-[Ring A]-[L¹]-[Ring B] in which Rings A and B are phenyl and Ring C is a heteroaryl ring. These intermediates are useful for making compounds of Formula II.

More specifically, Scheme XI describes the preparation of intermediates in which Ring C is an imidazole ring.

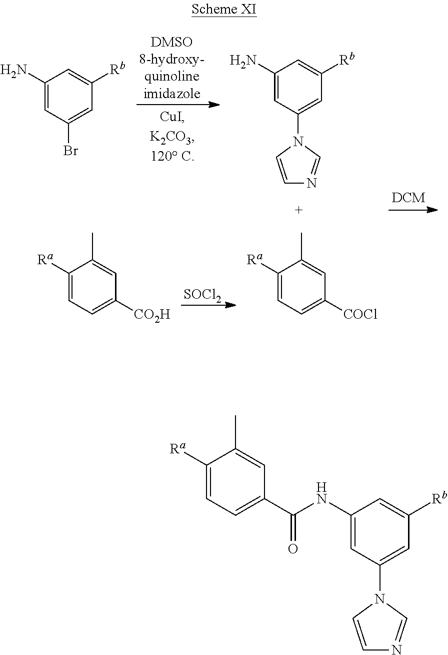

Scheme XII describes the preparation of intermediates in which Ring C is a pyrrole or an oxazole ring.

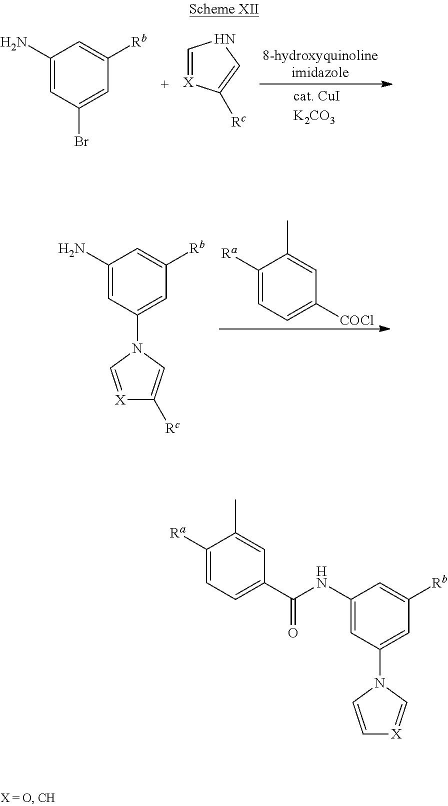

Scheme XIII illustrates the synthesis of W-[Ring A]-[L¹]-[Ring B] in which Rings A and B are phenyl and an R^{b} substituent is -L²-[Ring D]. These intermediates are useful for making compounds of Formula III in which Ring D is a 5 or 6-membered heterocycle, containing one or two heteroatoms.

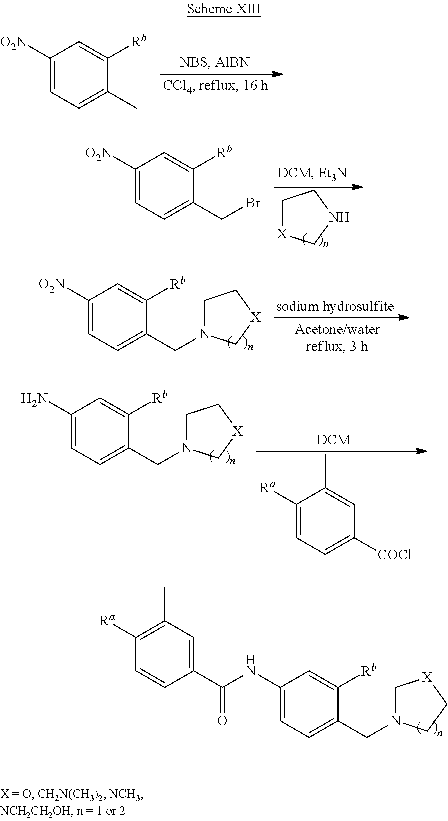

In this scheme, non-limiting examples of substituents R^{b} on Ring B are halo, e.g., Cl; lower alkyl groups, e.g., isopropyl; and substituted lower alkyl groups, e.g. —CF3; and non-limiting examples of Ring D are N,N-dimethylpyrrolidine, N-(2-hydroxyethyl)piperazine, and N-methylpiperazine.

Intermediates W-[Ring A]-[L¹]-[Ring B], such as those presented in the various synthetic schemes above, can be reacted with an acetylenic Ring T using the Sonogashira coupling conditions described in the general Scheme I.

An example is depicted below in Scheme XIV, in which Ring T moiety can be further derivatized after the Sonogashira coupling step, to generate various interesting substituted analogs of this invention.

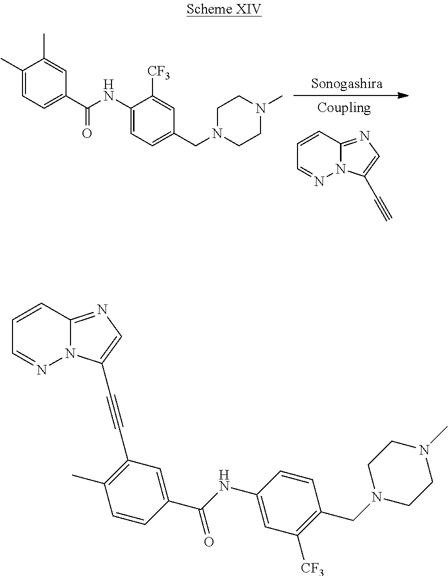

Alternatively, the W-[Ring A]-[L¹]-[Ring B] can be reacted under Sonogashira conditions with trimethylsilylacetylene, prior to the coupling with an iodo- or a bromo-activated Ring T as otherwise described in the general Scheme II.

An example is depicted in Scheme XV:

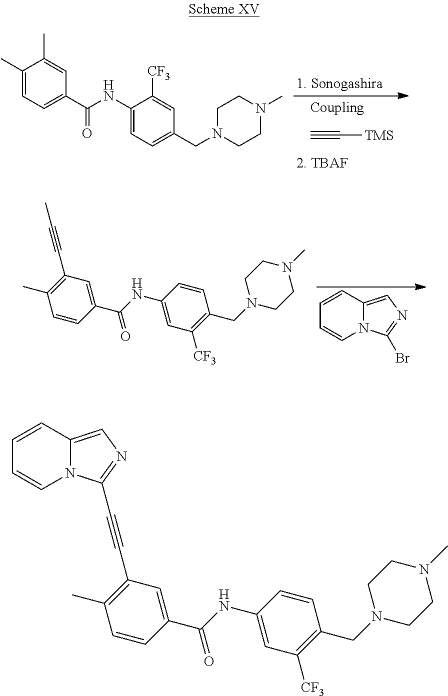

In other embodiments, the steps can be carried out in a different order. For example, the Sonogashira Coupling reaction can be used to Ring T to Ring A prior to linking that portion to Ring B and/or [Ring B]-[L²]-[Ring D] and/or [Ring B]-[Ring C] as shown in Scheme XVI.

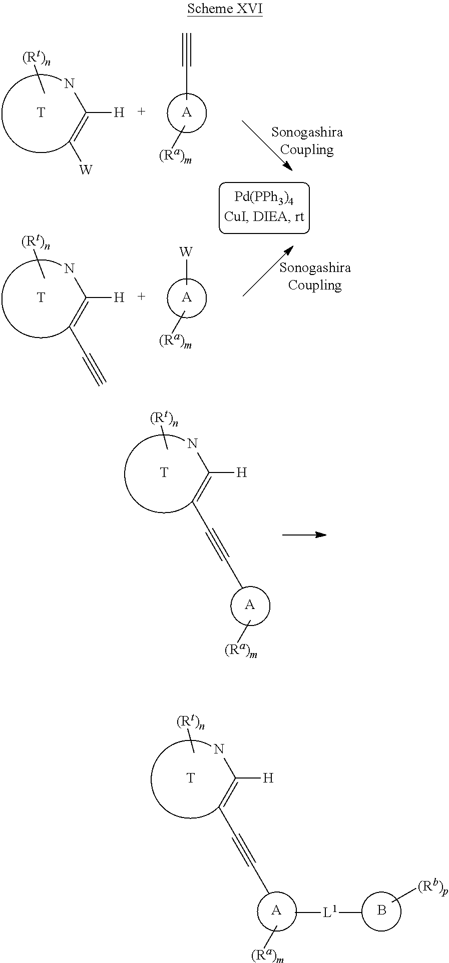

In a non-limiting example in which Ring A and Ring B are phenyl and L¹ is CONH, Scheme XVII describes Sonogashira Coupling of an acetylenic Ring T with 3-iodo-4-methylbenzoic acid (a Ring A moiety) to generate a [Ring T]-[Ring A] intermediate which then undergoes an amide coupling with an optionally substituted Ring B moiety:

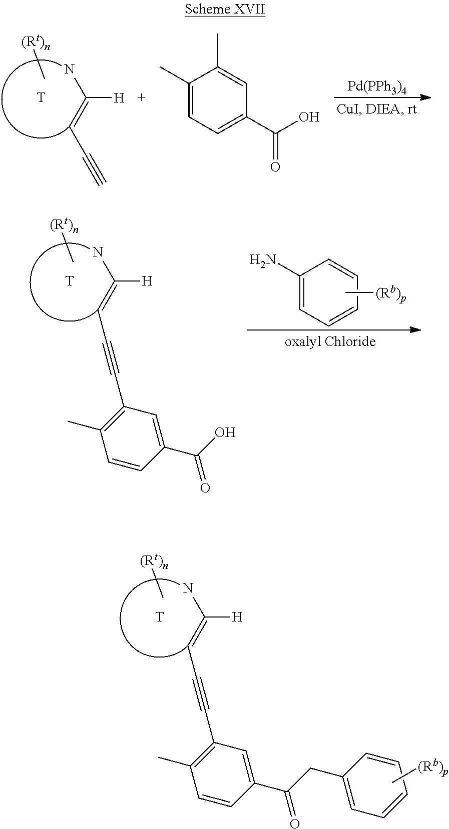

This approach is illustrated in Scheme XVIII which depicts the coupling of an acetylenic Ring T (i.e., 3-ethynylimidazo[1,2-b]pyridazine) with a substituted W-[Ring A] (i.e., 3-iodo-4-methylbenzoic acid), followed by an amide coupling of the resultant [Ring T]-[Ring A]-COON intermediate with a H2N-[Ring B]-L2-[Ring C] moiety (i.e., 4-((4-methylpiperazin-1-yl)methyl)-3-(trifluoromethylaniline):

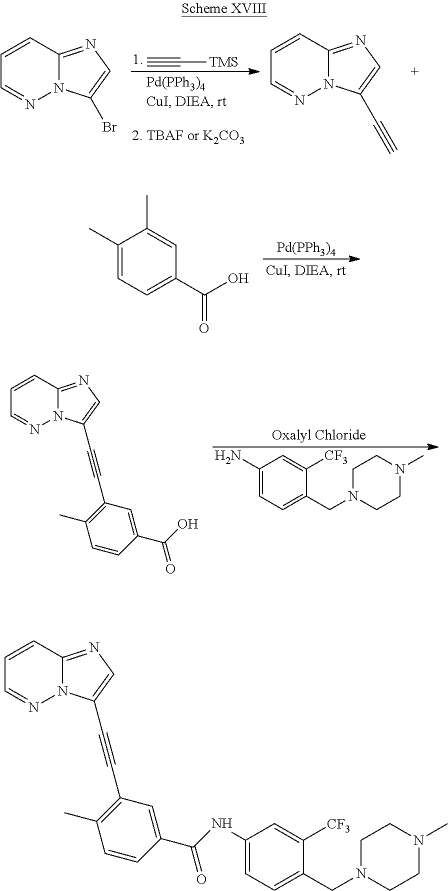

Alternatively, as another illustration of the practitioner's range of assembly options, the 3-iodo-4-methylbenzoic acid Ring A intermediate can be reacted in a Sonogashira reaction with trimethylsilylacetylene, which after silyl deprotection, can a second Sonogashira coupling reaction with an activated Ring T as illustrated in Scheme XIX.

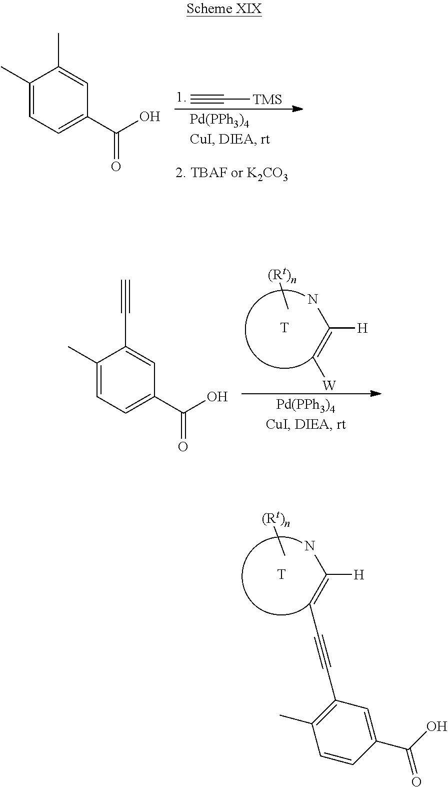

With synthetic approaches such as the foregoing, combined with the examples which follow, additional information provided herein and conventional methods and materials, the practitioner can prepare the full range of compounds disclosed herein.

In addition to the general synthetic approaches disclosed above, the synthesis of ponatinib free base and ponatinib hydrochloride have been specifically reported in Applicant's own WO 2011/053,938, which is incorporated here by reference. For the convenience of the reader, the synthetic scheme is reproduced immediately below.

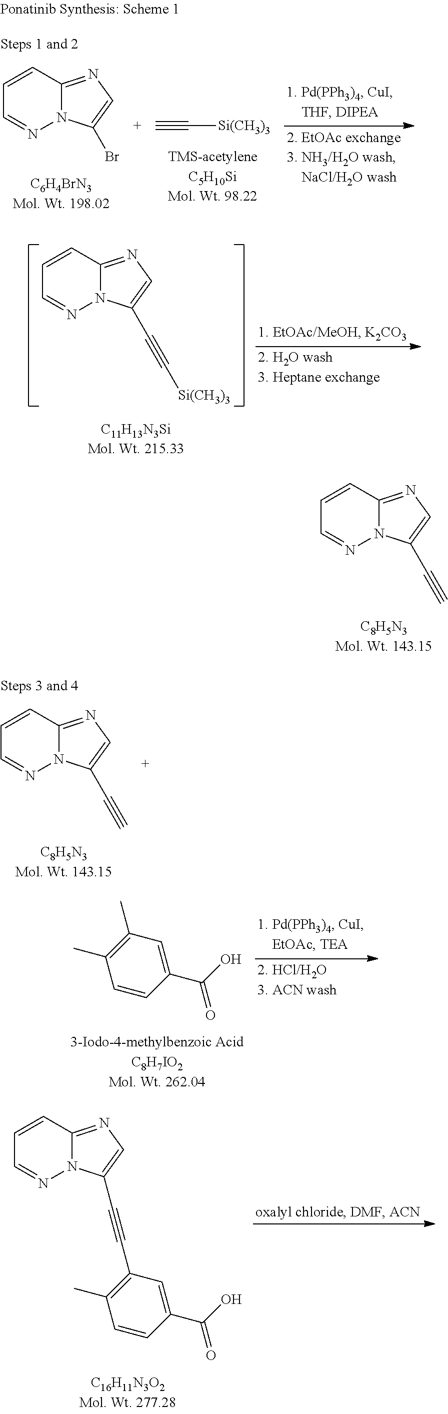

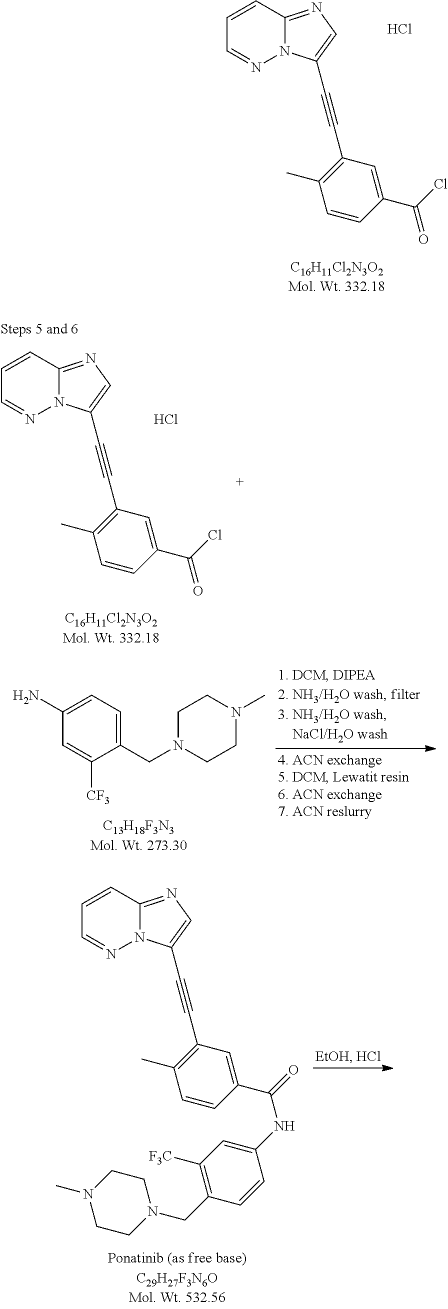 [SMILES: Cc1ccc(C(=O)Cl)cc1C#Cc1cnc2cccnn12 ; Cl ; Cc1ccc(C(=O)Cl)cc1C#Cc1cnc2cccnn12 ; Cl ; CN1CCN(Cc2ccc(N)cc2C(F)(F)F)CC1 ; Cc1ccc(C(=O)Nc2ccc(CN3CCN(C)CC3)c(C(F)(F)F)c2)cc1C#Cc1cnc2cccnn12]

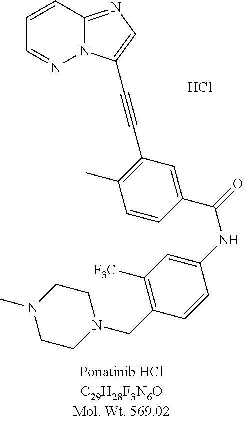 [SMILES: Cc1ccc(C(=O)Nc2ccc(CN3CCN(C)CC3)c(C(F)(F)F)c2)cc1C#Cc1cnc2cccnn12 ; Cl]

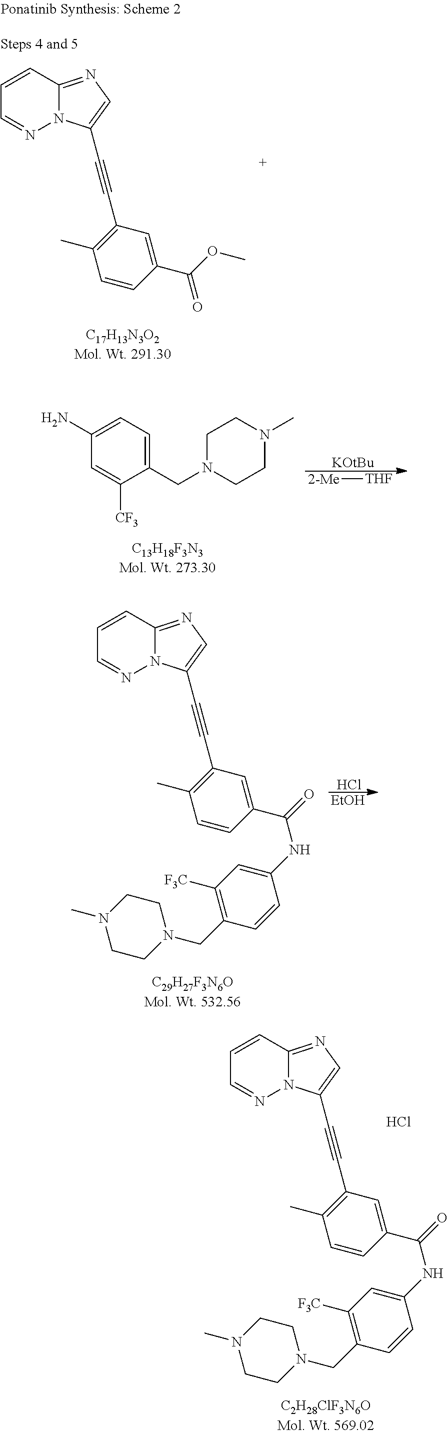

The mono-hydrochloride salt of ponatinib has been used for carrying out clinical trials. Further identifying information for ponatinib includes:

Chemical name: 3-(Imidazo[1,2-b]pyridazin-3-ylethynyl)-4-methyl-N-(4-((4-methylpiperazin-1-yl)methyl)-3-(trifluoromethyl)phenyl)benzamide, hydrochloride salt;

USAN: ponatinib;

USANM: ponatinib hydrochloride;

CAS Registry No.: 1114544-31-8 (HCl Salt) and 943319-70-8 (free base);

CAS Index name: Benzamide,3-(2-imidazo[1,2-b]pyridazin-3-ylethnyl)-4-methyl-N-[4-[(4-methyl-1-piperazinyl)methyl]-3-(trifluoromethyl)phenyl]-hydrochloride (1:1);

Molecular Formula: C₂₉H₂₈ClF₃N₆O(HCl salt) and C₂₉H₂₇F₃N₆O (free base) (no chiral centers); and

Molecular Weight: 569.02 g/mol (HCl salt) and 532.56 g/mol (free base).

### Exemplary Compounds

Some of the compounds described in the following examples have been converted into an HCl salt. The general procedure for generating HCl salts is described below:

To the final product was added just enough MeOH saturated with HCl (g) to dissolve, cooled to 0° C. for 0.5-1 h, filtered, washed solid with ice cold MeOH then Et₂O, and the resulting solid dried in a vacuum desiccator to provide in most cases the tris HCl salt.

### Example 1

### N-(3-(1H-imidazol-1-yl)-5-(trifluoromethyl)phenyl)-3-(imidazo[1,2-a]pyrazin-3-ylethynyl)-4-methylbenzamide

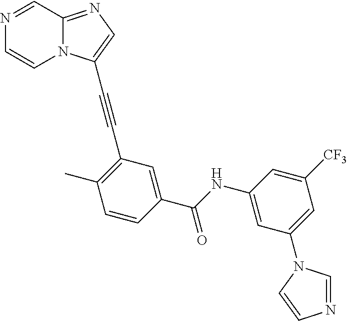 [SMILES: Cc1ccc(C(=O)Nc2cc(-n3ccnc3)cc(C(F)(F)F)c2)cc1C#Cc1cnc2cnccn12]

### Imidazo[1,2-a]pyrazine

A solution of aminopyrazine (1 g, 10.5 mmol) and chloroacetaldehyde (50% wt in H2O; 1.98 g, 12.6 mmol) in 1.6 mL of EtOH was heated at 90° C. in a sealed tube for 5 h. Upon cooling to ambient temperature, the reaction mixture was concentrated and diluted with dichloromethane (DCM). The organic layer washed with saturated aqueous NaHCO₃ then dried over MgSO₄ and concentrated. The crude product was purified by silica gel flash chromatography (eluted with 10% MeOH/DCM) to provide 0.8 g of product.

### 3-((Trimethylsilyl)ethynyl)imidazo[1,2-a]pyrazine

A mixture of 3-bromoimidazo[1,2-a]pyrazine (0.15 g, 0.76 mmol; prepared according to J. Bradac, et al. J. Org. Chem. (1977), 42, 4197-4201), 0.09 g (0.91 mmol) of ethynyltrimethylsilane, 0.044 g (0.038 mmol) of Pd(PPh₃)₄, 0.014 g (0.076 mmol) of CuI, and 0.26 mL (1.52 mmol) of diisopropylethylamine in 3.8 mL of DMF was heated at 50° C. overnight under an atmosphere of N₂. Upon cooling to ambient temperature, the reaction mixture was concentrated and the crude product was purified by silica gel flash chromatography (eluted with 50% EtOAc/hexanes) to provide 0.15 g of product: 216 m/z (M+H).

### 3-Ethynylimidazo[1,2-a]pyrazine

To a solution of 3-((Trimethylsilyl)ethynyl)imidazo[1,2-a]pyrazine (0.15 g, 0.7 mmol) in 3.5 mL of THF was added 1.05 mL (1.05 mmol) of tetrabutylammonium fluoride (1.0M in THF) at ambient temperature. The solution was stirred for 15 min, concentrated, and the crude product purified by silica gel flash chromatography (eluted with 50% EtOAc/hexanes) to provide 0.078 g of product.

### 3-(1H-imidazol-1-yl)-5-(trifluoromethyl)aniline

A mixture of 3-Amino-5-bromobenzotrifluoride (4.0 g, 0.0167 mol), 8-hydroxy quinoline (0.362 g, 0.0025 mol), CuI (0.476 g, 0.025 mol), imidazole (1.36 g, 0.0199 mol), and potassium carbonate (2.52 g, 0.0183 mol) in 17 mL of DMSO (degassed with argon for ˜10 min) was heated at 120° C. under an atmosphere of argon for 15 h; the HPLC indicated no starting material. A 14% aqueous solution of ammonium hydroxide was added to the cooled mixture and this was stirred for 1 h at ambient temperature. Water (50 mL) and EtOAc (200 mL) were added and the aqueous layer was extracted with EtOAc (3×30 mL). The combined organic layers were dried over Na₂SO₄ and concentrated. The crude product was purified by silica gel flash chromatography (eluted with EtOAc/hexanes) to provide 2.51 g of product.

### N-(3-(1H-imidazol-1-yl)-5-(trifluoromethyl)phenyl)-3-iodo-4-methylbenzamide

To 3-Iodo-4-methylbenzoic acid (3.07 g, 0.0117 mol) was added thionyl chloride (10 mL) and refluxed for 2 h. The excess thionyl chloride was carefully removed and the resulting acid chloride was dried in vacuo for 2 h. The residue was then dissolved in DCM (anhydrous, 25 mL) and cooled on ice. To the cooled solution was added 3-(1H-imidazol-1-yl)-5-(trifluoromethyl)aniline 5 (3.46 g, 0.0152 mol) in DCM followed by the dropwise addition of diisopropylethylamine (8.2 mL, 0.047 mol). This was stirred at ambient temperature for 21 h. The white solid that separated was filtered and washed with water and dried to provide 4.65 g of product. Additional product could be obtained from the filtrate following concentration and purification by silica gel flash chromatography in EtOAc/hexanes.

### N-(3-(1H-imidazol-1-yl)-5-(trifluoromethyl)phenyl)-3-(imidazo[1,2-a]pyrazin-3-ylethynyl)-4-methylbenzamide

A mixture of 3-Ethynylimidazo[1,2-a]pyrazine (0.075 g, 0.52 mmol), 0.245 g (0.52 mmol) of N-(3-(1H-imidazol-1-yl)-5-(trifluoromethyl)phenyl)-3-iodo-4-methylbenzamide, 0.030 g (0.026 mmol) of Pd(PPh₃)₄, 0.007 g (0.039 mmol) of CuI, and 0.14 mL (0.78 mmol) of diisopropylethylamine in 3.0 mL of DMF was stirred at ambient temperature overnight under an atmosphere of N₂. The reaction mixture was concentrated and the crude product was purified by silica gel flash chromatography (eluted with 10% EtOAc/hexanes, then 100% EtOAc, then 10% MeOH/EtOAc) to provide 0.090 g of product as a solid: 487 m/z (M+H).

### Alternative Synthesis of N-(3-(1H-imidazol-1-yl)-5-(trifluoromethyl)phenyl)-3-(imidazo[1,2-a]pyrazin-3-ylethynyl)-4-methylbenzamide

3-((Trimethylsilyl)ethynyl)imidazo[1,2-a]pyrazine can be prepared as described previously. In one variation, the reaction can also be carried out in THF instead of DMF. The crude product can also be purified by silica gel pad chromatography (eluted with ethyl acetate/hexane) and a brief treatment with activated charcoal (Darco) can be carried out to help further reduce contamination with the homo coupling product.

### 3-Ethynylimidazo[1,2-a]pyrazine

To a solution of 3-((trimethylsilyl)ethynyl)imidazo[1,2-a]pyrazine (1.39 mol) in 10× volume of Ethyl acetate and 1.5× volume of Methanol is added two and a half equivalents of potassium carbonate at ambient temperature and the solution stirred for 1 hour. Potassium carbonate is filtered off and the organic stream is washed with water and with saturated sodium chloride solution (two or more times). Aqueous phases can be combined and re-extracted with ethyl acetate. Organic streams can then be combined and concentrated under vacuum to about 0.5 L. Solids can be allowed to precipitate out upon concentration. Slurry is cooled, e.g. to about −5° C., stored overnight, filtered, and washed with about 0.3 L of cold ethyl acetate. The solids can then be dried under vacuum.

3-(imidazo[1,2-a]pyrazin-3-ylethynyl)-4-methylbenzoic acid can be prepared in a manner similar to that described above for the Sonogashira reaction. 3-Ethynylimidazo[1,2-a]pyrazine and 3-iodo-4-methylbenzoic acid are used as coupling partners. Alternatively, the solvent (DMF) can be replaced by ethyl acetate and the base (Hunig base) can be replaced by triethylamine. The product can be isolated by filtration of the crude reaction mixture. The filter cake is washed sequentially with a solvent such as ethyl acetate and then water, then dried in a vacuum oven. Further purification can be achieved by slurrying the solids in water adjusted to pH 3 with the addition of concentrated HCl. After filtration and water wash, the product can be dried in a vacuum oven.

### N-(3-(1H-imidazol-1-yl)-5-(trifluoromethyl)phenyl)-3-(imidazo[1,2-a]pyrazin-3-ylethynyl)-4-methylbenzamide

3-(imidazo[1,2-a]pyrazin-3-ylethynyl)-4-methylbenzoic acid (18 mmol) is dissolved in methylene chloride (100 mL). To this solution is added 3 equivalents of 4-methylmorpholine (NMM) followed by 1.05 equivalents of oxalyl chloride. After stirring at ambient temperature for 30 minutes, 0.8 equivalents of 3-(1H-imidazol-1-yl)-5-(trifluoromethyl)aniline (prepared as above) is added along with 5 mole % of DMAP. After initially stirring at ambient temperature, the mixture is brought to reflux and stirred overnight. After 16 h an additional 0.2 equivalents of the aniline is added, bringing the total charge to 1 equivalent. The mixture can then be stirred for an additional 2 h, quenched with water, and the layers separated. The aqueous layer can be extracted with methylene chloride (2×50 mL) and the combined extracts can be washed with water. The combined methylene chloride layers can then be evaporated and the residue dissolved in 100 mL of ethyl acetate (20 mL). After standing for 1 h, the product is allowed to crystallize. The mixture is cooled, e.g. to 0° C., filtered, and the solid product is washed with cold ethyl acetate.

### N-(3-(1H-imidazol-1-yl)-5-(trifluoromethyl)phenyl)-3-(imidazo[1,2-a]pyrazin-3-ylethynyl)-4-methylbenzamide mono hydrochloride salt

N-(3-(1H-imidazol-1-yl)-5-(trifluoromethyl)phenyl)-3-(imidazo[1,2-a]pyrazin-3-ylethynyl)-4-methylbenzamide (0.94 mmol) can be suspended in MeCN (10 ml) and heated with stirring to a temperature of 45 to 55° C. (hot plate temperature). Hydrochloric acid (1.1 eq 1M solution in EtOH) is added to obtain dissolution. Within a few minutes, a precipitate is allowed to form. The suspension can be cooled to ambient temperature and then filtered and washed with MeCN (1×1.5 ml liquors+1×1.5 ml fresh). The solid can be dried at 50° C. under vacuum to constant weight.

### Example 2

### 3-(Imidazo[1,2-a]pyrazin-3-ylethynyl)-4-methyl-N-(4-((4-methylpiperazin-1-yl)methyl)-3-(trifluoromethyl)phenyl)benzamide

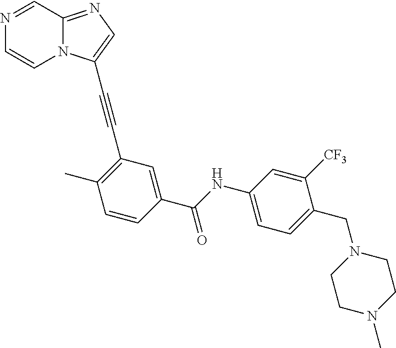 [SMILES: Cc1ccc(C(=O)Nc2ccc(CN3CCN(C)CC3)c(C(F)(F)F)c2)cc1C#Cc1cnc2cnccn12]

The title compound was synthesized from 3-ethynylimidazo[1,2-a]pyrazine and 3-iodo-4-methyl-N-(4-((4-methylpiperazin-1-yl)methyl)-3-(trifluoromethyl)phenyl)benzamide in a manner similar to that described for Example 1. The product was obtained as a solid: 533 m/z (M+H).

### 1-(Bromomethyl)-4-nitro-2-(trifluoromethyl)benzene

A suspension of 2-methyl-5-nitrobenzotrifluoride (3.90 g, 19 mmol), N-bromosuccinimide (NBS, 3.56 g, 20 mmol), 2,2′-azobis(2-methylpropionitrile) (AIBN, 94 mg, 0.6 mmol) in CCl₄ (40 mL) was refluxed under N₂ for 16 h. HPLC indicated ca. 50% conversion. More NBS (10 mmol) and AIBN (0.6 mmol) was added, and the mixture was refluxed for another 14 h. HPLC indicated ca. 80% conversion. The reaction mixture was cooled down, and the solid was filtered off and washed with EtOAc. The combined filtrate was washed with aq. NaHCO₃, dried over Na₂SO₄, filtered, concentrated on rotovap and further dried under vacuum. ¹H NMR shows the ratio of desired product to unreacted 2-methyl-5-nitrobenzotrifluoride is 75:25. This material was not purified but used directly in the next step.

### 1-Methyl-4-(4-nitro-2-(trifluoromethyl)benzyl)piperazine

To a solution of crude 1-(bromomethyl)-4-nitro-2-(trifluoromethyl)benzene (13.33 mmol, 75% pure) in DCM (10 mL) was added Et₃N (1.4 mL, 10 mmol) and 1-methylpiperazine (1.1 mL, 10 mmol). After stirring for 3 h at rt, aq. NaHCO₃ was added, and the mixture was extracted with DCM. The combined organic layer was dried over Na₂SO₄, filtered, concentrated, and the resulting residue was purified by silica gel chromatography (eluted with 10% MeOH/DCM) to provide 2.21 g of product as a pale yellow oil.

### 4-((4-Methylpiperazin-1-yl)methyl)-3-(trifluoromethyl)aniline

A suspension of 1-methyl-4-(4-nitro-2-(trifluoromethyl)benzyl)piperazine (1.23 g, 4 mmol) and sodium hydrosulfite (7.0 g, 85% pure from Aldrich, 40 mmol) in acetone and water (1:1, 20 mL) was refluxed for 3 h. Upon cooling, the volatile components (mainly acetone) were removed on rotavap, and the resulting mixture was subjected to filtration. The solid was thoroughly washed with EtOAc. The combined filtrate was extracted with n-BuOH (4×), and the combined organic layer was washed with saturated aq. NaHCO₃, dried (Na₂SO₄), filtered, concentrated, and the resulting residue was purified by silica gel chromatography (eluted with 5% MeOH/DCM, MeOH was pre-saturated with ammonia gas) to provide 0.71 g of product as a pale yellow solid.

### 3-Iodo-4-methyl-N-(4-((4-methylpiperazin-1-yl)methyl)-3-(trifluoromethyl)phenyl)Benzamide

3-Iodo-4-methylbenzoyl chloride (0.48 g, 1.7 mmol), prepared from the reaction of 3-iodo-4-methylbenzoic acid and SOCl₂ (as previously described), was added to a solution of 4-((4-methylpiperazin-1-yl)methyl)-3-(trifluoromethyl)aniline (0.47 g, 1.7 mmol), N,N-diisopropylethylamine (0.26 g, 2.0 mmol), and a catalytic amount of DMAP in THF (10 mL). After stirring at rt for 2 h, the reaction was quenched with water. EtOAc was added and the layers separated. The combined organic layers were concentrated to dryness and purified by silica gel chromatography (eluted with 5% MeOH/DCM, MeOH was pre-saturated with ammonia gas), to provide 0.51 g of product as an off-white solid.

### Alternative synthesis of 3-(Imidazo[1,2-a]pyrazin-3-ylethynyl)-4-methyl-N-(4-((4-methylpiperazin-1-yl)methyl)-3-(trifluoromethyl)phenyl)benzamide

3-(Imidazo[1,2-a]pyrazin-3-ylethynyl)-4-methyl-N-(4-((4-methylpiperazin-1-yl)methyl)-3-(trifluoromethyl)phenyl)benzamide and its mono hydrochloride salt can be prepared in an alternative synthesis similar to that described in Example 1 from 3-(imidazo[1,2-a]pyrazin-3-ylethynyl)-4-methylbenzoic acid and 4-((4-methylpiperazin-1-yl)methyl)-3-(trifluoromethyl)aniline (as prepared above).

### Example 3

### N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide

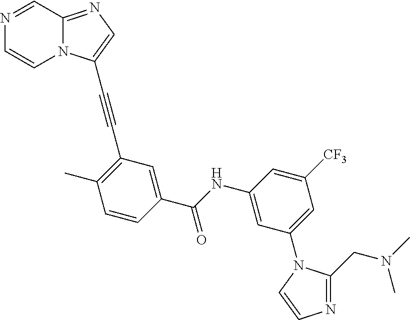 [SMILES: Cc1ccc(C(=O)Nc2cc(-n3ccnc3CN(C)C)cc(C(F)(F)F)c2)cc1C#Cc1cnc2cnccn12]

The title compound was synthesized from 3-ethynylimidazo[1,2-a]pyrazine and N-(3-(2-((dimethylamino)methyl)-1H-imidazol-1-yl)-5-(trifluoromethyl)phenyl)-3-iodo-4-methylbenzamide in a manner similar to that described for Example 1. The product was obtained as a solid: 544 m/z (M+H).

### 1-(1H-imidazol-2-yl)-N,N-dimethylmethanamine

To a two-necked round-bottomed flask equipped with a reflux condenser and a pressure-equalizing addition funnel, was added 2-imidazolecarboxaldehyde (6 g, 62.5 mmol) in MeOH (60 mL). To this suspension (ambient temperature) was added a solution of dimethylamine (40% aqueous, 60 mL) at a fast dropping rate (20 min). After the addition was complete, solid sodium borohydride (7 g, 186.8 mmol,) was CAUTIOUSLY added portionwise over 45 min. Foaming occurred after each portion, and the internal temperature was allowed to maintain ˜50° C. without external cooling. The reaction mixture was then heated to 65° C. for 3 h and allowed to cool to ambient temperature for overnight. The reaction contents were concentrated in vacuo and the resultant residue was taken up in EtOAc (2×30 mL) washed with brine and with CHCl₃ (4×100 mL). The EtOAc extract was discarded. The CHCl₃ extract was dried over (NaSO₄), filtered, and concentrated in vacuo to give 3.7 g of the desired product as a waxy solid.

### 3-(2-((Dimethylamino)methyl)-1H-imidazol-1-yl)-5-(trifluoromethyl)aniline

3-Amino-5-bromobenzotrifluoride (6 g, 25 mmol) and 1-(1H-imidazol-2-yl)-N,N-dimethylmethanamine (3.7 g, 29.6 mmol) were dissolved in anhydrous DMSO (25 mL). To this was added CuI (0.95 g, 7.5 mmol), 8-hydroxy quinoline (0.72 g, 7.5 mmol) and K₂CO₃ (6.9 g, 50 mmol). The mixture was stirred vigorously and degassed with N₂ for 15 minutes. The flask was then equipped with a condenser and heated at 120° C. for 18 h. The resultant heterogeneous mixture was cooled to rt, poured into 14% aq. NH₄OH (100 mL) and extracted with EtOAc (3×300 ml). The combined extracts were dried over NaSO₄ and concentrated in vacuo. The residue was chromatographed over silica gel eluting with MeOH/DCM (5:95) to furnish 3.5 g of the desired product as a tan colored material: 285 m/z (M+H).

### N-(3-(2-((dimethylamino)methyl)-1H-imidazol-1-yl)-5-(trifluoromethyl)phenyl)-3-iodo-4-methylbenzamide

3-Iodo-4-methylbenzoyl chloride (2.2 g, 7.88 mmol), dissolved in anhydrous THF (13 mL), was added dropwise to a solution of 3-(2-((dimethylamino)methyl)-1H-imidazol-1-yl)-5-(trifluoromethyl)aniline (1.5 g, 5.5 mmol), DIPEA (2.1 mL, 11.8 mmol) in THF (30 mL) at ˜5° C. The resultant solution was stirred at ambient temperature overnight. The solvent was removed in vacuo and the crude residue was redissolved in CH₂Cl₂ and washed with 1N NaOH. The organic layer was then washed with water, and brine then dried over NaSO₄ before being concentrated in vacuo. The brown colored residue was then triturated in a mixture of hexanes/DCM to precipitate 1.4 g of the desired product as an off-white powder: 529 m/z (M+H).

### Alternative Synthesis of N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide

N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide and its mono hydrochloride salt can be prepared in an alternative synthesis similar to that described in Example 1 from 3-(imidazo[1,2-a]pyrazin-3-ylethynyl)-4-methylbenzoic acid and 3-(2-((Dimethylamino)methyl)-1H-imidazol-1-yl)-5-(trifluoromethyl)aniline (as prepared above).

### Example 4

### 3-(Imidazo[1,2-a]pyridin-3-ylethynyl)-4-methyl-N-(3-(4-methyl-1H-imidazol-1-yl)-5-(trifluoromethyl)phenyl)benzamide

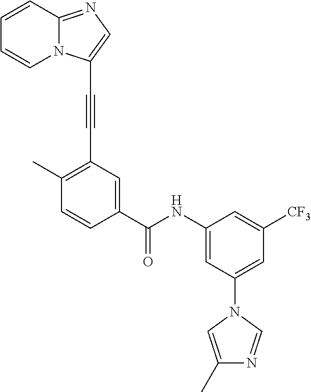 [SMILES: Cc1cn(-c2cc(NC(=O)c3ccc(C)c(C#Cc4cnc5ccccn45)c3)cc(C(F)(F)F)c2)cn1]

### 3-Ethynylimidazo[1,2-a]pyridine

To 3-bromoimidazo[1,2-a]pyridine (5 g, 0.0254 mol) in acetonitrile (50 mL) in a sealed tube was added bis(triphenylphosphine) palladium(II) dichloride (0.445 g, 0.634 mmol), CuI (0.17 g, 0.89 mmol), dicyclohexylamine (5.6 mL, 0.028 mol) and ethynyltrimethylsilane (7.2 mL, 0.051 mol). The solution was purged with argon for 15 minutes, sealed and heated at 80° C. for 3 h. At this point the HPLC did not show any starting bromide. The solvents were concentrated and to the residue was added water and dichloromethane (25 mL each). The organic layer was separated and the aqueous layer was repeatedly extracted with dichloromethane (3×20 mL). The combined extracts were dried (Na₂SO₄), and concentrated (Rf, 0.47 in 1/1 hexanes/ethyl acetate). The resulting residue was dissolved in THF (100 mL) and treated with tetrabutyl ammonium fluoride monohydrate (8.3 g, 0.032 mol) in water (5 mL) and the mixture was stirred at rt for 2 h. The solvents were concentrated and the resulting residue was partitioned between water (25 mL) and dichloromethane (150 mL). The aqueous layer was extracted with dichloromethane (2×30 mL). The combined extracts were dried (Na₂SO₄), and concentrated. The resulting residue was purified by combiflash on silica gel using hexanes/ethyl acetate. The desired product was eluted with 50/50 hexane/ethyl acetate and isolated as an off-white solid: MS (M+H)⁺ 200.

### 3-(4-Methyl-1H-imidazol-1-yl)-5-(trifluoromethyl)aniline

A suspension of 3-bromo-5-(trifluoromethyl)aniline (4.8 g, 20 mmol), 4-methylimidazole (1.97 g, 24 mmol), potassium carbonate (3.04 g, 22 mmol), CuI (0.57 g, 3 mmol), and 8-hydroxyquinoline (0.44 g, 3 mmol,) in dry DMSO (20 mL) in a pressure tube was degassed by bubbling N₂ into the suspension for 10 minutes while stirring. The tube was sealed tightly. The mixture was heated at 120° C. (oil bath temperature) for 15 h. The mixture was cooled down to 45-50° C. and 14% aq. NH₄OH (20 mL) was added. The mixture was maintained at this temperature for 1 h. After cooling to rt, water and ethyl acetate were added. The aqueous layer was extracted with ethyl acetate and the combined organic layers were passed through a short silica gel column to remove most of green/blue Cu salts. The filtrate was dried over sodium sulfate and concentrated on a rotavap. The crude product was recrystallized from EtOAc/hexanes, giving pure pale yellow needles. The mother liquor was concentrated and the residue was purified on silica gel column (5% methanol/methylene chloride), yielding a second crop as pale yellow needles.

### 3-Iodo-4-methyl-N-(3-(4-methyl-1H-imidazol-1-yl)-5-(trifluoromethyl)phenyl)Benzamide

3-Iodo-4-methylbenzoic acid (2.62 g, 10 mmol) was refluxed in SOCl₂ (10 mL) for 1 h. The volatile components were removed on a rotavap and the residue was dissolved in benzene (10 mL), concentrated to dryness on a rotavap and further dried under vacuum. The resulting acyl chloride was added to a solution 3-(4-methyl-1H-imidazol-1-yl)-5-(trifluoromethyl)benzeneamine (2.46 g, 10.2 mmol), N,N-diisopropylethylamine (1.56 g, 12 mmol), and a catalytic amount of DMAP in THF (20 mL). After stirring at rt for 2 h, the reaction was quenched with water. EtOAc was added and the layers separated. The combined organic layers were concentrated to dryness and used without purification in next step.

### 3-(Imidazo[1,2-a]pyridin-3-ylethynyl)-4-methyl-N-(3-(4-methyl-1H-imidazol-1-yl)-5-(trifluoromethyl)phenyl)benzamide

To a solution of 3-iodo-4-methyl-N-(3-(4-methyl-1H-imidazol-1-yl)-5-(trifluoromethyl)phenyl)benzamide (0.11 g, 0.22 mmol.) in DMF (1 mL) in a sealed tube was added Pd[(PPh₃)₄] (0.013 g, 0.011 mmol), CuI (3 mg, 0.016 mmol), diethylisopropylamine (0.057 mL, 0.33 mmol.), followed by 3-ethynylimidazo[1,2-a]pyridine (0.040 g, 0.28 mmol.). The mixture was purged with argon for 15 minutes, sealed and stirred at rt for 28 h. The solvent was concentrated and the residue was taken up in methylene chloride (50 mL). The organic layer was washed with water, dried (Na₂SO₄) and evaporated to leave a brown residue which was purified by combiflash (hexane/ethyl acetate/methanol) to yield the desired material: MS (M+H)⁺ 500.

### Alternative Synthesis of 3-(Imidazo[1,2-a]pyridin-3-ylethynyl)-4-methyl-N-(3-(4-methyl-1H-imidazol-1-yl)-5-(trifluoromethyl)phenyl)benzamide

3-(Imidazo[1,2-a]pyridin-3-ylethynyl)-4-methyl-N-(3-(4-methyl-1H-imidazol-1-yl)-5-(trifluoromethyl)phenyl)benzamide and its mono hydrochloride salt can be prepared in an alternative synthesis similar to that described in Example 1 from 3-(imidazo[1,2-a]pyridin-3-ylethynyl)-4-methylbenzoic acid and 3-(4-Methyl-1H-imidazol-1-yl)-5-(trifluoromethyl)aniline (as prepared above). The 3-(imidazo[1,2-a]pyridin-3-ylethynyl)-4-methylbenzoic acid is prepared in a manner similar to that described in Example 1 using 3-Ethynylimidazo[1,2-a]pyridine and 3-iodo-4-methylbenzoic acid as Sonogashira coupling partners.

### Example 5

### N-(3-(1H-imidazol-1-yl)-5-(trifluoromethyl)phenyl)-3-(imidazol-[1,2-a]pyridin-3-ylethynyl)-4-methylbenzamide

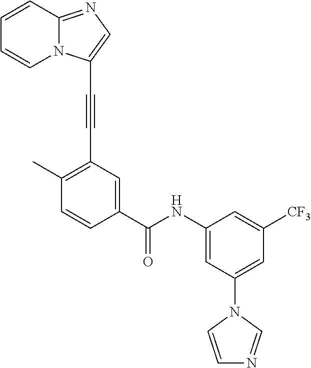 [SMILES: Cc1ccc(C(=O)Nc2cc(-n3ccnc3)cc(C(F)(F)F)c2)cc1C#Cc1cnc2ccccn12]

The titled compound was made as for example 1 using N-(3-(1H-imidazol-1-yl)-5-(trifluoromethyl)phenyl)-3-iodo-4-methylbenzamide and 3-ethynylimidazo[1,2-a]pyridine: MS (M+H)⁺ 486. The titled compound can also be prepared according to the alternative synthesis described in example 1 from 3-(imidazo[1,2-a]pyridin-3-ylethynyl)-4-methylbenzoic acid and 3-(1H-imidazol-1-yl)-5-(trifluoromethyl)aniline (as prepared in Example 1). The 3-(imidazo[1,2-a]pyridin-3-ylethynyl)-4-methylbenzoic acid is prepared in a manner similar to that described in Example 1 using 3-Ethynylimidazo[1,2-a]pyridine and 3-iodo-4-methylbenzoic acid as Sonogashira coupling partners.

### Example 6

### 3-(Imidazo[1,2-a]pyridin-3-ylethynyl)-4-methyl-N-(4-(trifluoromethyl)pyridin-2-yl)benzamide

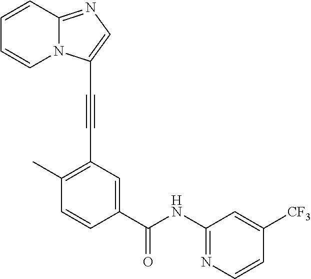 [SMILES: Cc1ccc(C(=O)Nc2cc(C(F)(F)F)ccn2)cc1C#Cc1cnc2ccccn12]

The titled compound was made as for example 1 using 3-iodo-4-methyl-N-(4-(trifluoromethyl)pyridin-2-yl)benzamide and 3-ethynylimidazo[1,2-a]pyridine: MS (M+H)⁺ 421.39.

### Example 7

### N-(5-tert-butylisoxazol-3-yl)-3-(imidazo[1,2-a]pyridin-3-ylethynyl)-4-methylbenzamide

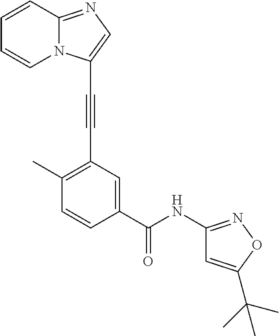 [SMILES: Cc1ccc(C(=O)Nc2cc(C(C)(C)C)on2)cc1C#Cc1cnc2ccccn12]

The titled compound was made as for example 1 using N-(5-tert-butylisoxazol-3-yl)-3-iodo-4-methylbenzamide and 3-ethynylimidazo[1,2-a]pyridine: MS (M+H)⁺ 399.

### Example 8

### 3-(Imidazo[1,2-a]pyridin-3-ylethynyl)-4-methyl-N-(4-((4-methylpiperazin-1-yl)methyl)-3-(trifluoromethyl)phenyl)benzamide

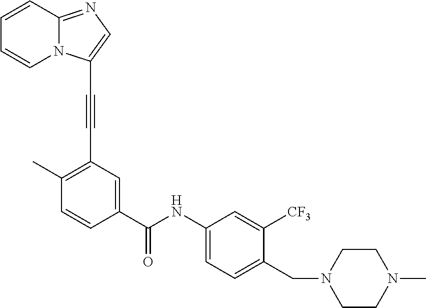 [SMILES: Cc1ccc(C(=O)Nc2ccc(CN3CCN(C)CC3)c(C(F)(F)F)c2)cc1C#Cc1cnc2ccccn12]

3-Ethynylimidazo[1,2-a]pyridine (37 mg, 0.26 mmol), 3-iodo-4-methyl-N-(4-((4-methylpiperazin-1-yl)methyl)-3-(trifluoromethyl)phenyl)benzamide (103.4 mg, 0.2 mmol), (prepared as in Example 2), Pd[(PPh₃)₄] (11.6 mg, 5 mol %), and CuI (2.9 mg, 7.5 mmol %) was placed in a vial with rubber septum. The mixture underwent 3 cycles of vacuum/filling with N₂, and DMF (1.5 ml) and N,N-diisopropylethylamine (53 mL, 0.3 mmol) was added. The mixture was stirred at rt for 16 h, and the reaction was quenched with H₂O. EtOAc and more water were added for extraction. The combined organic layer was dried (Na₂SO₄), filtered, concentrated, and the resulting residue was purified by silica gel chromatography (eluent: 5% MeOH in methylene chloride, MeOH was pre-saturated with ammonia gas), giving the titled compound as an off-white solid (53%, 56 mg): MS (M+H)⁺ 532.

### Alternative Synthesis of 3-(Imidazo[1,2-a]pyridin-3-ylethynyl)-4-methyl-N-(4-((4-methylpiperazin-1-ylmethyl)-3-(trifluoromethyl)phenyl)benzamide

3-(Imidazo[1,2-a]pyridin-3-ylethynyl)-4-methyl-N-(4-((4-methylpiperazin-1-yl)methyl)-3-(trifluoromethyl)phenyl)benzamide and its mono hydrochloride salt can be prepared in an alternative synthesis similar to that described in Example 1 from 3-(imidazo[1,2-a]pyridin-3-ylethynyl)-4-methylbenzoic acid and 4-((4-methylpiperazin-1-yl)methyl)-3-(trifluoromethyl)aniline (as prepared in example 2). The 3-(imidazo[1,2-a]pyridin-3-ylethynyl)-4-methylbenzoic acid is prepared in a manner similar to that described in Example 1 using 3-Ethynylimidazo[1,2-a]pyridine and 3-iodo-4-methylbenzoic acid as Sonogashira coupling partners.

### Example 9

### N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide

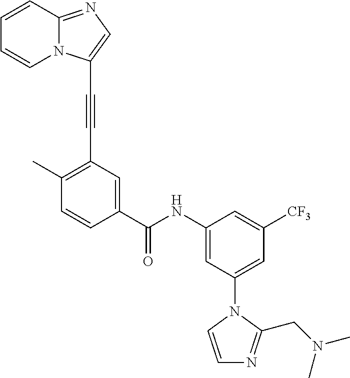 [SMILES: Cc1ccc(C(=O)Nc2cc(-n3ccnc3CN(C)C)cc(C(F)(F)F)c2)cc1C#Cc1cnc2ccccn12]

To 3-ethynylimidazo[1,2-a]pyridine (0.032 g, 0.22 mmol) in anhydrous DMF (1.26 mL) was added N-(3-(2-((dimethylamino)methyl)-1H-imidazol-1-yl)-5-(trifluoromethyl)phenyl)-3-iodo-4-methylbenzamide (prepared as in Example 3), Pd(PPh₃)₄ (0.013 g, 0.011 mmol), CuI (0.0032 mg, 0.0165 mmol) and DIPEA (0.064 mL, 0.44 mmol). The solution was degassed with argon for 15 minutes then stirred overnight at rt. The solvent was removed and the resultant residue was chromatographed over silica gel eluting initially with EtOAc and then with methanol/methylene chloride (5:95) to furnish the desired product: (0.07 g, 59%) MS (M+H)⁺ 542.

### Alternative Synthesis of N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide

N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide and its mono hydrochloride salt can be prepared in an alternative synthesis similar to that described in Example 1 from 3-(imidazo[1,2-a]pyridin-3-ylethynyl)-4-methylbenzoic acid and 3-(2-((Dimethylamino)methyl)-1H-imidazol-1-yl)-5-(trifluoromethyl)aniline (as prepared in Example 3). The 3-(imidazo[1,2-a]pyridin-3-ylethynyl)-4-methylbenzoic acid is prepared in a manner similar to that described in Example 1 using 3-Ethynylimidazo[1,2-a]pyridine and 3-iodo-4-methylbenzoic acid as Sonogashira coupling partners.

### Example 10

### 3-((8-Acetamidoimidazo[1,2-a]pyridin-3-yl)ethynyl)-4-methyl-N-(4-(trifluoromethyl)pyridin-2-yl)benzamide

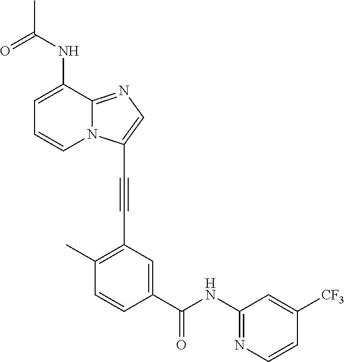 [SMILES: CC(=O)Nc1cccn2c(C#Cc3cc(C(=O)Nc4cc(C(F)(F)F)ccn4)ccc3C)cnc12]

### N-(3-Ethynylimidazo[1,2-a]pyridin-8-yl)acetamide

N-(3-Ethynylimidazo[1,2-a]pyridin-8-yl)acetamide was synthesized as for example 1A from N-(3-bromoimidazo[1,2-a]pyridin-8-yl)acetamide (E. Smakula Hand and William W. Paudler, J. Org. Chem., 1978, 43, 2900-2906). The titled compound was isolated as an off-white solid, Rf, 0.6 (hexane/ethylacetate 50/50): MS (M+H)⁺ 200.

### 3-((8-Acetamidoimidazo[1,2-a]pyridin-3-yl)ethynyl)-4-methyl-N-(4-(trifluoromethyl)pyridin-2-yl)benzamide

The titled compound was made as for example 1 using 3-iodo-4-methyl-N-(4-(trifluoromethyl)pyridin-2-yl)benzamide and N-(3-ethynylimidazo[1,2-a]pyridin-8-yl)acetamide: MS (M+H)⁺ 478.4.

### Example 11

### N-(3-(1H-imidazol-1-yl)-5-(trifluoromethyl)phenyl)-3-((8-acetamidoimidazo[1,2-a]pyridin-3-yl)ethynyl)-4-methylbenzamide

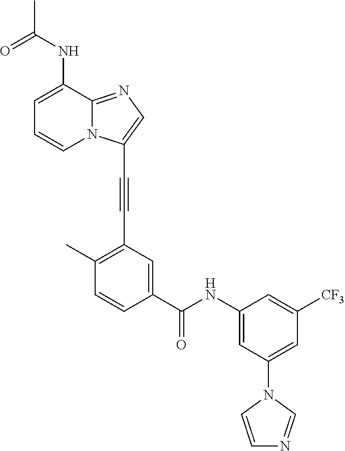 [SMILES: CC(=O)Nc1cccn2c(C#Cc3cc(C(=O)Nc4cc(-n5ccnc5)cc(C(F)(F)F)c4)ccc3C)cnc12]

The titled compound was made as for example 10 using N-(3-(1H-imidazol-1-yl)-5-(trifluoromethyl)phenyl)-3-iodo-4-methylbenzamide and N-(3-ethynylimidazo[1,2-a]pyridin-8-yl)acetamide: MS (M+H) 543.

### Example 12

### 4-Methyl-3-((8-(4-(methylsulfonyl)phenylamino)imidazo[1,2-a]pyridin-3-yl)ethynyl)-N-(4-(trifluoromethyl)pyridin-2-yl)benzamide

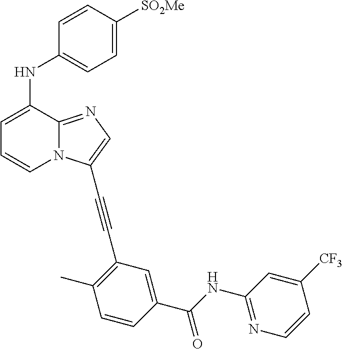 [SMILES: Cc1ccc(C(=O)Nc2cc(C(F)(F)F)ccn2)cc1C#Cc1cnc2c(Nc3ccc(S(C)(=O)=O)cc3)cccn12]

### 8-(Benzyloxy)-3-bromoimidazo[1,2-a]pyridine

To a solution of 2-amino-3-benzyloxypyridine (25.0 g, 124.9 mmol) and chloroacetaldehyde (50% wt in H₂O; 16.7 mL, 131.2 mmol) in 250 mL of EtOH was heated at reflux in a sealed tube for 19 h. Upon cooling to ambient temperature, the reaction mixture was concentrated and the resulting brown oil added 125 mL 1N NaOH then extracted with dichloromethane (DCM). The combined organic layers were washed with H₂O, dried over Na₂SO₄ and concentrated. Upon concentrating the solution, a tan solid formed which was filtered and dried to provide 25.8 g of crude product.

To a solution of crude 8-(benzyloxy)imidazo[1,2-a]pyridine (8.73 g, 38.9 mmol) in 100 mL of EtOH was added, dropwise, 4.8 mL (46.7 mmol) of a solution of 1:1 Br₂/H₂O at ambient temperature under an atmosphere of N₂. The resulting dark orange suspension was stirred at ambient temperature for 30 min, added 60 mL 1N NaOH, and the reaction mixture extracted with DCM. The combined organic layers were dried over Na₂SO₄ and concentrated. The crude product was purified by silica gel flash chromatography (eluted with 30% EtOAc/hexanes) to provide 7.04 g of product.

### 8-(Benzyloxy)-3-((trimethylsilyl)ethynyl)imidazo[1,2-a]pyridine

A mixture of 8-(benzyloxy)-3-bromoimidazo[1,2-a]pyridine (10.0 g, 33.0 mmol), 9.39 mL (66.0 mmol) of ethynyltrimethylsilane, 0.580 g (0.825 mmol) of Pd(PPh₃)₂Cl₂, 0.230 g (1.19 mmol) of CuI, and 5.09 mL (36.3 mmol) of diisopropylamine in 100 mL of acetonitrile was heated at reflux for 3 h under an atmosphere of N₂. Upon cooling to ambient temperature, the reaction mixture was concentrated and the crude product was purified by silica gel flash chromatography (eluted with 20-50% EtOAc/hexanes) to provide 6.74 g of product: 321 m/z (M+H).

### 3-((Trimethylsilyl)ethynyl)imidazo[1,2-a]pyridin-8-yl trifluoromethanesulfonate

To a cooled (0° C.) solution of 8-(benzyloxy)-3-((trimethylsilyl)ethynyl)imidazo[1,2-a]pyridine (3.44 g, 10.7 mmol) in 400 mL of DCM, under an atmosphere of N₂, was added via cannulation 100 mL (100 mmol) of boron trichloride (1.0M solution in hexanes). The reaction solution was stirred at 0° C./N₂ for 30 min, to which was added (0° C.) 200 mL H₂O followed by extraction with DCM. The combined organic layers were washed with brine, dried over Na₂SO₄ and concentrated. The crude product was purified by silica gel flash chromatography (eluted with 30% EtOAc/hexanes then 10% MeOH/DCM) to provide 2.32 g of deprotected product: 231 m/z (M+H).

To a cooled (−78° C.) solution of 8-(hydroxy)-3-((trimethylsilyl)ethynyl)imidazo[1,2-a]pyridine (2.32 g, 10.1 mmol) and 1.63 mL (20.1 mmol) of pyridine in 50 mL of DCM, under an atmosphere of N₂, was added 2.03 mL (12.1 mmol) of trifluoromethanesulfonic anhydride via syringe. Upon removing the cooling bath, the reaction solution was stirred at ambient temperature (N₂) for 2 h. The reaction mixture was poured into a stirring solution of 100 mL 1.0N HCl, the layers separated, and the organic layer washed successively with 1.0N HCl, H₂O, saturated aqueous NaHCO₃, and brine. The organic layer was dried over Na₂SO₄ and concentrated. The crude product was filtered through a small plug of silica gel (eluted with 30% EtOAc/hexanes), concentrated, and further dried in vacuo to provide 3.63 g of product: 363 m/z (M+H).

### N-(4-(Methylsulfonyl)phenyl)-3-((trimethylsilyl)ethynyl)imidazo[1,2-c]pyridin-8-amine

A mixture of 3-((trimethylsilyl)ethynyl)imidazo[1,2-a]pyridin-8-yl trifluoromethanesulfonate (0.329 g, 0.91 mmol), 0.186 (1.09 mmol) of 4-(methylsulfonyl)aniline, 0.083 g (0.091 mmol) of Pd₂(dba)₂, 0.087 g (0.181 mmol) of 2-dicyclohexylphosphino-2′,4′,6′-triisopropylbiphenyl, and 0.385 g (1.81 mmol) of potassium phosphate in 8 mL of DME was heated at 80° C. in a sealed tube overnight under an atmosphere of N₂. Upon cooling to ambient temperature, the reaction mixture was concentrated and the crude product was purified by silica gel flash chromatography (triethylamine-treated silica gel; eluted with 0-80% EtOAc/hexanes) to provide 0.058 g of product: 384 m/z (M+H).

### 3-Ethynyl-N-(4-(methylsulfonyl)phenyl)imidazo[1,2-a]pyridin-8-amine

To a solution of N-(4-(methylsulfonyl)phenyl)-3-((trimethylsilyl)ethynyl)imidazo[1,2-a]pyridin-8-amine (0.058 g, 0.15 mmol) in 1.5 mL of THF was added 0.23 mL (0.23 mmol) of tetrabutylammonium fluoride (1.0M in THF) at ambient temperature. The solution was stirred for 15 min, concentrated, and the crude product purified by silica gel flash chromatography (triethylamine-treated silica gel; eluted with 100% DCM then 5% MeOH/DCM) to provide a quantitative yield (0.047 g) of product: 312 m/z (M+H).

### 4-Methyl-3-((8-(4-(methylsulfonyl)phenylamino)imidazo[1,2-a]pyridin-3-yl)ethynyl)-N-(4-(trifluoromethyl)pyridin-2-yl)benzamide

A mixture of 3-ethynyl-N-(4-(methylsulfonyl)phenyl)imidazo[1,2-a]pyridin-8-amine 5 (0.048 g, 0.154 mmol), 0.069 g (0.170 mmol) of 3-iodo-4-methyl-N-(4-(trifluoromethyl)pyridin-2-yl)benzamide, 0.009 g (0.008 mmol) of Pd(PPh₃)₄, 0.002 g (0.012 mmol) of CuI, and 0.04 mL (0.23 mmol) of diisopropylethylamine in 0.8 mL of DMF was stirred at ambient temperature overnight under an atmosphere of N₂. The reaction mixture was concentrated and the crude product was purified by silica gel flash chromatography (triethylamine-treated silica gel; eluted with 10% EtOAc/hexanes to 100% EtOAc) to provide 0.047 g of product as a solid: 590 m/z (M+H).

### Example 13

### 4-methyl-3-((8-(4-sulfamoylphenylamino)imidazo[1,2-a]pyridin-3-yl)ethynyl)-N-(4-(trifluoromethyl)pyridin-2-yl)benzamide

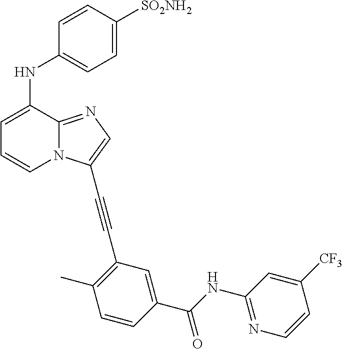 [SMILES: Cc1ccc(C(=O)Nc2cc(C(F)(F)F)ccn2)cc1C#Cc1cnc2c(Nc3ccc(S(N)(=O)=O)cc3)cccn12]

The title compound was synthesized from 3-ethynyl-N-(4-sulfamoylphenyl)imidazo[1,2-a]pyridin-8-amine and 3-iodo-4-methyl-N-(4-(trifluoromethyl)pyridin-2-yl)benzamide in a manner similar to that described for Example 12. The product was obtained as a solid: 591 m/z (M+H).

### Example 14

### (R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide

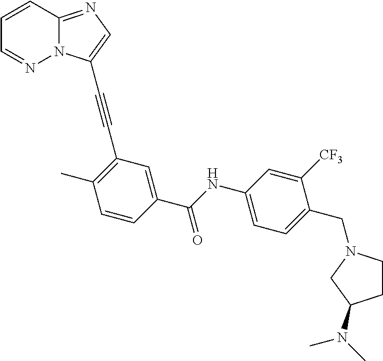 [SMILES: Cc1ccc(C(=O)Nc2ccc(CN3CC[C@@H](N(C)C)C3)c(C(F)(F)F)c2)cc1C#Cc1cnc2cccnn12]

### 3-((Trimethylsilyl)ethynyl)imidazo[1,2-b]pyridazine

A mixture of 3-bromoimidazo[1,2-b]pyridazine (36.78 g, 0.186 mol; prepared according to Stanovnik, B. et al. Synthesis (1981), 12, 987-989), ethynyltrimethylsilane (21.89 g, 0.223 mol), Pd(PPh₃)₄ (10.73 g, 9.29 mmol), CuI (5.30 g, 0.028 mol), and diisopropylethylamine (32.4 mL, 0.279 mol) in 150 mL of DMF was stirred at ambient temperature, under an atmosphere of N₂, for 1 h. The reaction mixture was concentrated and the crude product was purified by silica gel flash chromatography (eluted with 0-5% MeOH/DCM) to provide 28.46 g of product.

### 3-Ethynylimidazo[1,2-b]pyridazine

To a solution of 3-((trimethylsilyl)ethynyl)imidazo[1,2-b]pyridazine (28.46 g, 0.132 mol) in 200 mL of THF was added 145 mL (0.145 mol) of tetrabutylammonium fluoride (1.0M in THF) at ambient temperature. The solution was stirred for 15 min, concentrated, and the crude product purified by silica gel flash chromatography (eluted with 0-5% MeOH/DCM) to provide 17.84 g of product.

### 1-(Bromomethyl)-4-nitro-2-(trifluoromethyl)benzene

A suspension of 2-methyl-5-nitrobenzotrifluoride (3.90 g, 19 mmol), N-bromosuccinimide (NBS, 3.56 g, 20 mmol), and 2,2′-azobis(2-methylpropionitrile) (AIBN, 0.094 g, 0.6 mmol) in 40 mL of CCl₄ was heated at reflux under N₂ for 16 h. HPLC indicated ca. 50% conversion. Additional NBS (10 mmol) and AIBN (0.6 mmol) were added and the mixture was heated at reflux for another 14 h. HPLC indicated ca. 80% conversion. The reaction mixture was cooled to ambient temperature, and the solid was filtered and washed with EtOAc. The combined filtrate was washed with aq. NaHCO₃, dried over Na₂SO₄, filtered, concentrated on rotovap, and further dried under vacuum. ¹H NMR indicated the ratio of desired product to unreacted 2-methyl-5-nitrobenzotrifluoride to be 75:25. This material was used directly in the next step.

### (R)—N,N-Dimethyl-1-(4-nitro-2-(trifluoromethyl)benzyl)pyrrolidin-3-amine

To a solution of crude 1-(bromomethyl)-4-nitro-2-(trifluoromethyl)benzene (17.5 mmol, 75% pure) in 40 mL of DCM was added Et₃N (2.69 mL, 19.3 mmol) and (R)-(+)-3-(dimethylamino)pyrrolidine (2.0 g, 17.5 mmol). After stirring overnight at ambient temperature under an atmosphere of N₂, the reaction solution was concentrated, added aq. NaHCO₃ (100 mL), and the resulting mixture extracted with DCM (4×50 mL). The combined organic layer was dried over Na₂SO₄, filtered, concentrated, and the resulting residue was purified by silica gel chromatography (eluted with 0-10% MeOH/DCM) to provide 3.35 g of product as a yellow oil.

### (R)-1-(4-Amino-2-(trifluoromethyl)benzyl)-N,N-dimethylpyrrolidin-3-amine

To a solution of (R)—N,N-dimethyl-1-(4-nitro-2-(trifluoromethyl)benzyl)pyrrolidin-3-amine (1.20 g, 3.79 mmol) in 20 mL of wet EtOH was added 0.26 g of Pd/C (10% Pd on C) and the mixture shaken in a Parr apparatus (pressure reaction vessel purged thoroughly with H₂ and pressure regulated at 45 psi throughout) for 2-3 h. The reaction mixture was filtered through a small pad of celite, washed with EtOAc, and the combined organics concentrated to provide a quantitative yield of a light yellow oil. This material was used directly in the next step.

### (R)—N-(4-((3-(Dimethylamino)pyrrolidin-1-yl)methyl)-3-(trifluoromethyl)phenyl)-3-iodo-4-methylbenzamide

To a cooled (0° C.) solution of (R)-1-(4-amino-2-(trifluoromethyl)benzyl)-N,N-dimethylpyrrolidin-3-amine (3.79 mmol) in 14 mL DCM, under an atmosphere of N₂, was added 3-Iodo-4-methylbenzoyl chloride (1.17 g, 4.17 mmol; CAS#52107-98-9, prepared from the reaction of 3-iodo-4-methylbenzoic acid and SOCl₂) followed by dropwise addition of N,N-diisopropylethylamine (2.64 mL, 15.2 mmol). After stirring to ambient temperature over 1.5 h, the reaction mixture was concentrated and the crude product was purified by silica gel chromatography (eluted with 0-8% MeOH/DCM; MeOH was pre-saturated with ammonia gas), to provide 0.71 g of product as a thick yellow oil.

### (R)—N-(4-((3-(dimethylamino)pyrrolidin-1-yl)methyl)-3-(trifluoromethyl)phenyl)-3-(imidazo[1,2-b]pyridazin-3-ylethynyl)-4-methylbenzamide

A mixture of 3-ethynylimidazo[1,2-b]pyridazine (0.051 g, 0.34 mmol), 0.150 g (0.28 mmol) of (R)—N-(4-((3-(dimethylamino)pyrrolidin-1-yl)methyl)-3-(trifluoromethyl)phenyl)-3-iodo-4-methylbenzamide, 0.016 g (0.014 mmol) of Pd(PPh₃)₄, 0.004 g (0.021 mmol) of CuI, and 0.09 mL (0.51 mmol) of N,N-diisopropylethylamine in 3.5 mL of DMF was stirred at ambient temperature, under an atmosphere of N₂, for 3 days (reaction pushed to completion with additional equivalents of reagents and heating to 80° C.). The reaction mixture was concentrated and the crude product was purified by silica gel chromatography (eluted with 0-10% MeOH/DCM; MeOH was pre-saturated with ammonia gas) to provide 0.020 g of product as a solid: 547 m/z (M+H).

### Alternative Synthesis of (R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide

(R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide and its mono hydrochloride salt can be prepared in an alternative synthesis similar to that described in Example 1 from 3-(imidazo[1,2-b]pyridazin-3-ylethynyl)-4-methylbenzoic acid and (R)-1-(4-Amino-2-(trifluoromethyl)benzyl)-N,N-dimethylpyrrolidin-3-amine (as prepared above). The 3-(imidazo[1,2-b]pyridazin-3-ylethynyl)-4-methylbenzoic acid is prepared in a manner similar to that described in Example 1 using 3-Ethynylimidazo[1,2-b]pyridazine and 3-iodo-4-methylbenzoic acid as Sonogashira coupling partners.

### Example 15

### N-(3-(Imidazo[1,2-b]pyridazin-3-ylethynyl)-4-methylphenyl)-4-((4-methylpiperazin-1-yl)methyl)-3-(trifluoromethyl)benzamide

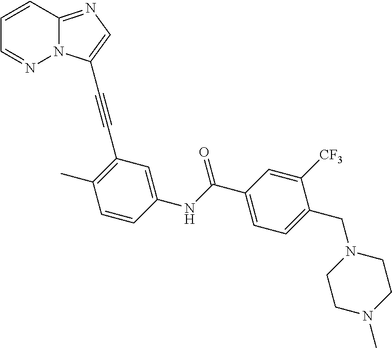 [SMILES: Cc1ccc(NC(=O)c2ccc(CN3CCN(C)CC3)c(C(F)(F)F)c2)cc1C#Cc1cnc2cccnn12]

The title compound was synthesized from 3-ethynylimidazo[1,2-b]pyridazine and N-(3-iodo-4-methylphenyl)-4-(4-methylpiperazin-1-yl)methyl)-3-(trifluoromethyl)benzamide in a manner similar to that described for Example 14. The product was obtained as a solid: 533 m/z (M+H).

### N-(3-Iodo-4-methylphenyl)-4-((4-methylpiperazin-1-yl)methyl)-3-(trifluoromethyl)benzamide

To a flask containing 1.0 g (2.67 mmol) of 4-[(4-methyl-1-piperazinyl)methyl]-3-(trifluoromethyl)-benzoic acid (CAS#859027-02-4; prepared according to Asaki, T. et al. Bioorg. Med. Chem. Lett. (2006), 16, 1421-1425), 0.62 g (2.67 mmol) of 3-Iodo-4-methylaniline, 0.77 g (4.0 mmol) of N-(3-dimethylaminopropyl)-N′-ethylcarbodiimide hydrochloride (EDAC), and 0.43 g (3.2 mmol) of N-hydroxybenzotriazole monohydrate (HOBt.H₂O) was added 5 mL of DCM and 5 mL of triethylamine. The solution was stirred at ambient temperature under an atmosphere of N₂ for 3 days, concentrated, and the crude product purified by silica gel chromatography (eluted with 100% EtOAc then 10% MeOH/EtOAc), to provide 0.69 g of product as a white solid.

### Example 16

### 3-(Imidazo[1,2-b]pyridazin-3-ylethynyl)-4-methyl-N-(4-((4-methylpiperazin-1-yl)methyl)-3-(trifluoromethyl)phenyl)benzamide

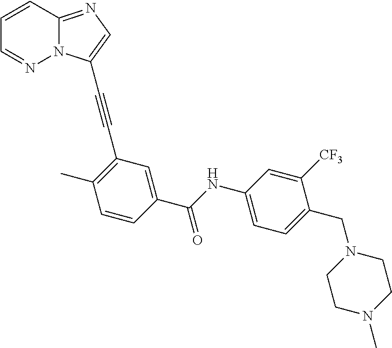 [SMILES: Cc1ccc(C(=O)Nc2ccc(CN3CCN(C)CC3)c(C(F)(F)F)c2)cc1C#Cc1cnc2cccnn12]

The title compound was synthesized in a manner similar to that described for Example 14, from 3-ethynylimidazo[1,2-b]pyridazine and 3-iodo-4-methyl-N-(4-((4-methylpiperazin-1-yl)methyl)-3-(trifluoromethyl)phenyl)benzamide (Prepared as described in Example 2). The product was obtained as a solid: 533 m/z (M+H).

### Alternative Synthesis of 3-(Imidazo[1,2-b]pyridazin-3-ylethynyl)-4-methyl-N-(4-((4-methylpiperazin-1-yl)methyl)-3-(trifluoromethyl)phenyl)benzamide

3-(Imidazo[1,2-b]pyridazin-3-ylethynyl)-4-methyl-N-(4-((4-methylpiperazin-1-yl)methyl)-3-(trifluoromethyl)phenyl)benzamide and its mono hydrochloride salt can be prepared in an alternative synthesis similar to that described in Example 1 from 3-(imidazo[1,2-b]pyridazin-3-ylethynyl)-4-methylbenzoic acid and 4-((4-methylpiperazin-1-yl)methyl)-3-(trifluoromethyl)aniline (as prepared in example 2). The 3-(imidazo[1,2-b]pyridazin-3-ylethynyl)-4-methylbenzoic acid is prepared in a manner similar to that described in Example 1 using 3-Ethynylimidazo[1,2-b]pyridazine and 3-iodo-4-methylbenzoic acid as Sonogashira coupling partners.

### Example 17

### N-(3-Chloro-4-((4-methylpiperazin-1-yl)methyl)phenyl)-3-(imidazol-[1,2-b]pyridazin-3-ylethynyl)-4-methylbenzamide

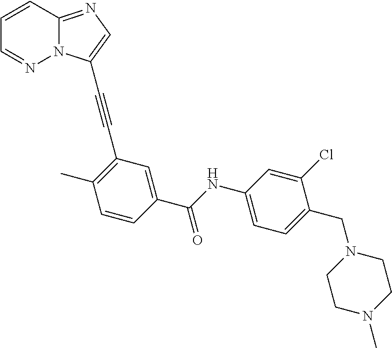 [SMILES: Cc1ccc(C(=O)Nc2ccc(CN3CCN(C)CC3)c(Cl)c2)cc1C#Cc1cnc2cccnn12]

The title compound was synthesized according to Example 14, from 3-ethynylimidazo[1,2-b]pyridazine and N-(3-chloro-4-((4-methylpiperazin-1-yl)methyl)phenyl)-3-iodo-4-methylbenzamide. The product was obtained as a solid: 499 m/z (M+H).

### 1-(Bromomethyl)-2-chloro-4-nitro-benzene

A suspension of 2-chloro-4-nitrotoluene (10.0 g, 58.3 mmol), N-bromosuccinimide (NBS, 10.9 g, 61.2 mmol), and 2,2′-azobis(2-methylpropionitrile) (AIBN, 0.29 g, 1.75 mmol) in 120 mL of CCl₄ was heated at reflux under an atmosphere of N₂ for 12 h. The reaction mixture was cooled to ambient temperature, and the solid was filtered and washed with EtOAc. The combined filtrate was washed with aq. NaHCO₃, dried over Na₂SO₄, filtered, concentrated on rotovap, and further dried under vacuum. ¹H NMR indicated the ratio of desired product to unreacted 2-chloro-4-nitrotoluene to be 50:50. This material was used directly in the next step.

### 1-(2-Chloro-4-nitrobenzyl)-4-methylpiperazine

To a solution of crude 1-(bromomethyl)-2-chloro-4-nitro-benzene (29.1 mmol; 50% pure) in 30 mL of DCM was added Et₃N (4.2 mL, 30 mmol) and 1-methylpiperazine (3.4 mL, 30 mmol). After stirring for 3 h at ambient temperature, aq. NaHCO₃ was added and the mixture was extracted with DCM. The combined organic layer was dried over Na₂SO₄, filtered, concentrated, and the resulting residue was purified by silica gel chromatography (eluted with 5% MeOH/DCM) to provide 6.80 g of product as a dark yellow oil.

### 3-Chloro-4-((4-methylpiperazin-1-yl)methyl)aniline

To a solution of 1-(2-chloro-4-nitrobenzyl)-4-methylpiperazine (0.96 g, 3.6 mmol) in MeOH/water (4:1, 50 mL) was added 1.80 g (33.7 mmol) of NH₄Cl and 1.47 g (26.3 mmol) of Fe dust and the mixture heated at reflux under an atmosphere of N₂ for 2 h (HPLC indicated no progress). To this was added 4 mL of glacial acetic acid and the mixture heated at reflux for an additional 2 h. The reaction mixture was cooled to ambient temperature, filtered, and the filtrate concentrated. The residue was partitioned between EtOAc and saturated aq. NaHCO₃, the separated aqueous layer was extracted with EtOAc, and the combined organics washed with brine and dried over Na₂SO₄. Upon concentration, the crude product was purified by silica gel chromatography (eluted with 5-7% MeOH/DCM; silica gel deactivated with 1% triethylamine/DCM) to provide 0.53 g of product.

### Alternative Synthesis of N-(3-Chloro-4-((4-methylpiperazin-1-yl)methyl)phenyl)-3-(imidazo[1,2-b]pyridazin-3-ylethynyl)-4-methylbenzamide

N-(3-Chloro-4-((4-methyl piperazin-1-yl)methyl)phenyl)-3-(imidazo[1,2-b]pyridazin-3-ylethynyl)-4-methylbenzamide and its mono hydrochloride salt can be prepared in an alternative synthesis similar to that described in Example 1 from 3-(imidazo[1,2-b]pyridazin-3-ylethynyl)-4-methylbenzoic acid and 3-Chloro-4-((4-methylpiperazin-1-yl)methyl)aniline (as prepared above). The 3-(imidazo[1,2-b]pyridazin-3-ylethynyl)-4-methylbenzoic acid is prepared in a manner similar to that described in Example 1 using 3-Ethynylimidazo[1,2-b]pyridazine and 3-iodo-4-methylbenzoic acid as Sonogashira coupling partners.

### Example 18

### N-(3-Cyclopropyl-4-((4-methylpiperazin-1-yl)methyl)phenyl)-3-(imidazo[1,2-b]pyridazin-3-ylethynyl)-4-methylbenzamide

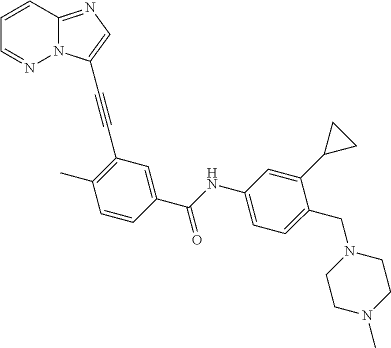 [SMILES: Cc1ccc(C(=O)Nc2ccc(CN3CCN(C)CC3)c(C3CC3)c2)cc1C#Cc1cnc2cccnn12]

The title compound was synthesized from 3-ethynylimidazo[1,2-b]pyridazine and N-(3-cyclopropyl-4-((4-methylpiperazin-1-yl)methyl)phenyl)-3-iodo-4-methylbenzamide in a manner similar to that described for Example 14 (nitro reduction performed in a manner similar to that described for Example 17; 0.25M in MeOH/10% AcOH). The product was obtained as a solid: 505 m/z (M+H).

### 1-(2-Cyclopropyl-4-nitrobenzyl)-4-methylpiperazine

A mixture of 1-(2-bromo-4-nitrobenzyl)-4-methylpiperazine (0.94 g, 3.0 mmol), 0.77 g (9.0 mmol) of cyclopropylboronic acid, 0.067 g (0.30 mmol) of Pd(OAc)₂, 2.87 g (13.5 mmol) of K₃PO₄, and 0.168 g (0.60 mmol) of tricyclohexylphosphine in 18 mL of toluene/water (5:1) was heated at reflux under an atmosphere of N₂ for 19 h. The reaction mixture was concentrated and the crude product was purified by silica gel chromatography (eluted with 5% MeOH/DCM; MeOH was pre-saturated with ammonia gas) to provide 0.80 g of product.

### Example 19

### 3-(Imidazo[1,2-b]pyridazin-3-ylethynyl)-N-(4-((4-methylpiperazin-1-yl)methyl)-3-(trifluoromethyl)phenyl)benzamide

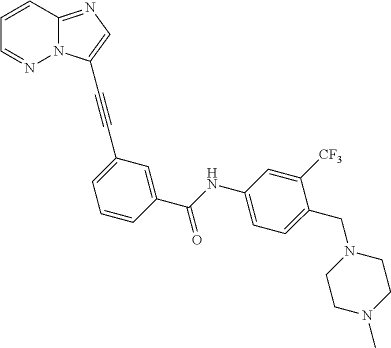 [SMILES: CN1CCN(Cc2ccc(NC(=O)c3cccc(C#Cc4cnc5cccnn45)c3)cc2C(F)(F)F)CC1]

The title compound was synthesized from 3-ethynylimidazo[1,2-b]pyridazine and 3-iodo-N-(4-((4-methylpiperazin-1-yl)methyl)-3-(trifluoromethyl)phenyl)benzamide in a manner similar to that described for Example 14. The product was obtained as a solid: 519 m/z (M+H).

The titled compound can also be prepared according to the alternative synthesis described in example 1 from 3-(imidazo[1,2-b]pyridazin-3-ylethynyl)-4-methylbenzoic acid and 4-((4-methylpiperazin-1-yl)methyl)-3-(trifluoromethyl)aniline (as prepared in example 2). The 3-(imidazo[1,2-b]pyridazin-3-ylethynyl)-4-methylbenzoic acid is prepared in a manner similar to that described in Example 1 using 3-Ethynylimidazo[1,2-b]pyridazine and 3-iodo-4-methylbenzoic acid as Sonogashira coupling partners.

### Example 20

### N-(4-((4-(2-Hydroxyethyl)piperazin-1-yl)methyl)-3-(trifluoromethyl)phenyl)-3-(imidazo[1,2-b]pyridazin-3-ylethynyl)-4-methylbenzamide

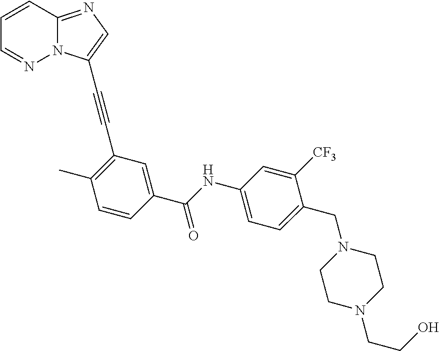 [SMILES: Cc1ccc(C(=O)Nc2ccc(CN3CCN(CCO)CC3)c(C(F)(F)F)c2)cc1C#Cc1cnc2cccnn12]

The title compound was synthesized from 3-ethynylimidazo[1,2-b]pyridazine and N-(4-((4-(2-hydroxyethyl)piperazin-1-yl)methyl)-3-(trifluoromethyl)phenyl)-3-iodo-4-methylbenzamide in a manner similar to that described for Example 14. The product was obtained as a solid: 563 m/z (M+H).

### Example 21

### 3-(Imidazo[1,2-b]pyridazin-3-ylethynyl)-4-methyl-N-(4-(piperazin-1-ylmethyl)-3-(trifluoromethyl)phenyl)benzamide

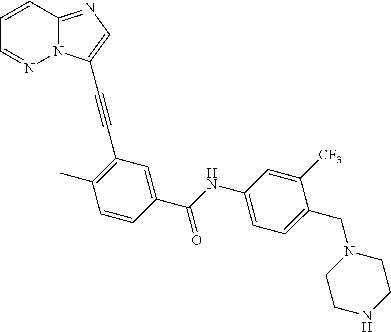 [SMILES: Cc1ccc(C(=O)Nc2ccc(CN3CCNCC3)c(C(F)(F)F)c2)cc1C#Cc1cnc2cccnn12]

The title compound was synthesized from 3-ethynylimidazo[1,2-b]pyridazine and tert-butyl-4-(4-(3-iodo-4-methylbenzamido)-2-(trifluoromethyl)benzyl)piperazine-1-carboxylate in a manner similar to that described for Example 14. Following deprotection using saturated MeOH/HCl (g), the product was obtained as a tris HCl salt: 519 m/z (M+H).

### Representative Biological Data

Compounds of this invention were evaluated in a variety of assays to determine their biological activities. For example, the compounds of the invention were tested for their ability to inhibit various kinases of interest. Some of the compounds tested displayed potent nanomolar activity against certain of the following kinases: A-RAF, B-RAF and C-RAF.

Kinase Inhibition Assay:

In vitro kinase inhibition assays to determine drug 1050 (the concentration that inhibits activity by 50%) were performed under the direction of applicants at Reaction Biology Corporation (Malvern, Pa.). Compounds were tested at 10 μM ATP using a 10-point curve with 3-fold serial dilutions starting at 1 μM.

The following examples shown in Table 1 were found to be potent C-RAF kinase inhibitors (IC50<5 μM):

TABLE 1
C-Raf inhibitors:

| | | C-RAF |
|---|---|---|
| | | (IC50 |
| Example No. | Structure | nM) |
| 2 | 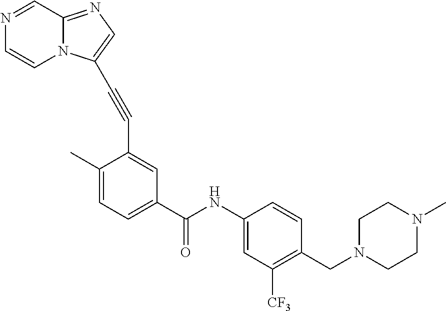 [SMILES: Cc1ccc(C(=O)Nc2ccc(CN3CCN(C)CC3)c(C(F)(F)F)c2)cc1C#Cc1cnc2cnccn12] | 2.126 |
| 8 | 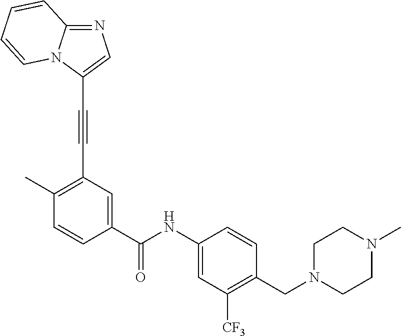 [SMILES: Cc1ccc(C(=O)Nc2ccc(CN3CCN(C)CC3)c(C(F)(F)F)c2)cc1C#Cc1cnc2ccccn12] | 26.01 |
| 10 | 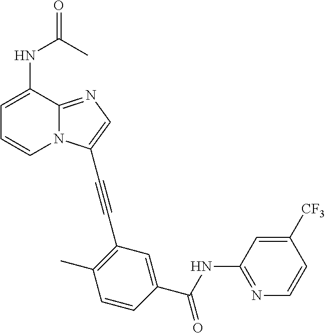 [SMILES: CC(=O)Nc1cccn2c(C#Cc3cc(C(=O)Nc4cc(C(F)(F)F)ccn4)ccc3C)cnc12] | 2.421 |
| 15 | 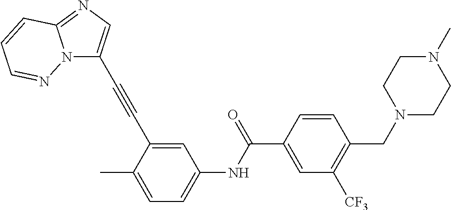 [SMILES: Cc1ccc(NC(=O)c2ccc(CN3CCN(C)CC3)c(C(F)(F)F)c2)cc1C#Cc1cnc2cccnn12] | 12.22 |

In addition, the compounds of Formula I shown in Table 2 were made in accordance with the methods provided by Examples 1-21 and were found to be potent C-RAF kinase inhibitors (IC50<5 μM):

TABLE 2
C-Raf inhibitors:

| | C-RAF |
|---|---|
| | (IC50 |
| Structure | nM) |
| 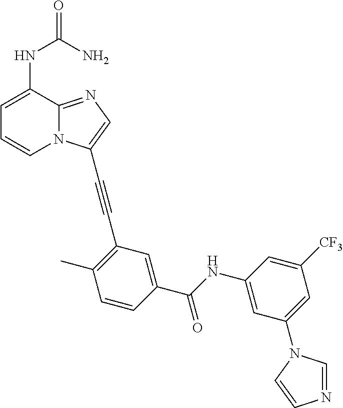 [SMILES: Cc1ccc(C(=O)Nc2cc(-n3ccnc3)cc(C(F)(F)F)c2)cc1C#Cc1cnc2c(NC(N)=O)cccn12] | 12.67 |
| 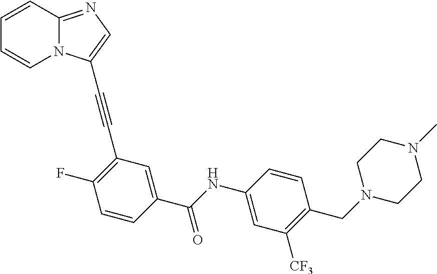 [SMILES: CN1CCN(Cc2ccc(NC(=O)c3ccc(F)c(C#Cc4cnc5ccccn45)c3)cc2C(F)(F)F)CC1] | 51.75 |
| 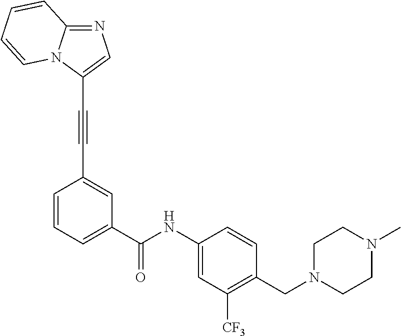 [SMILES: CN1CCN(Cc2ccc(NC(=O)c3cccc(C#Cc4cnc5ccccn45)c3)cc2C(F)(F)F)CC1] | 26.72 |
| 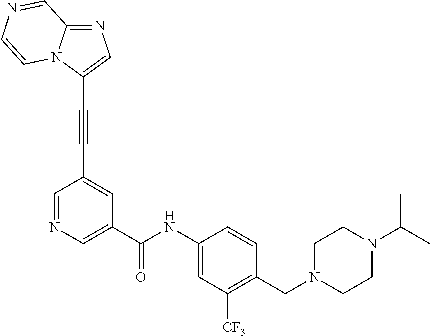 [SMILES: CC(C)N1CCN(Cc2ccc(NC(=O)c3cncc(C#Cc4cnc5cnccn45)c3)cc2C(F)(F)F)CC1] | 14.41 |
| 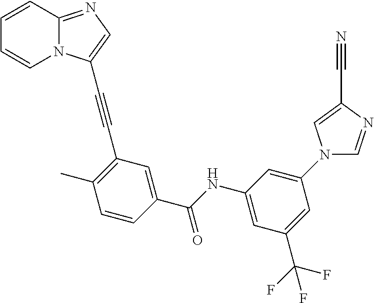 [SMILES: Cc1ccc(C(=O)Nc2cc(-n3cnc(C#N)c3)cc(C(F)(F)F)c2)cc1C#Cc1cnc2ccccn12] | 998.1 |
| 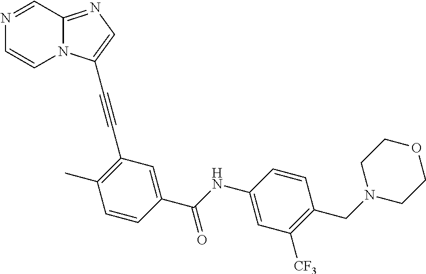 [SMILES: Cc1ccc(C(=O)Nc2ccc(CN3CCOCC3)c(C(F)(F)F)c2)cc1C#Cc1cnc2cnccn12] | 5.427 |
| 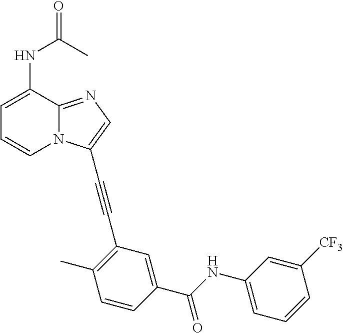 [SMILES: CC(=O)Nc1cccn2c(C#Cc3cc(C(=O)Nc4cccc(C(F)(F)F)c4)ccc3C)cnc12] | 7.3 |
| 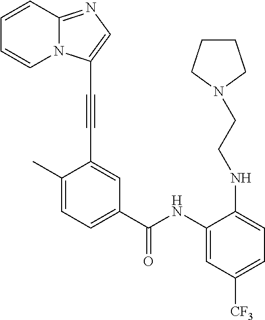 [SMILES: Cc1ccc(C(=O)Nc2cc(C(F)(F)F)ccc2NCCN2CCCC2)cc1C#Cc1cnc2ccccn12] | 24.34 |
| 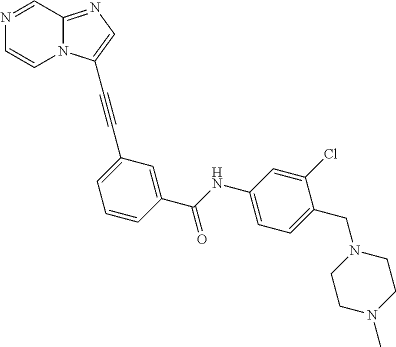 [SMILES: CN1CCN(Cc2ccc(NC(=O)c3cccc(C#Cc4cnc5cnccn45)c3)cc2Cl)CC1] | 3.961 |
| 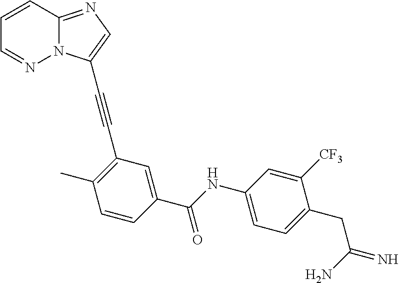 [SMILES: Cc1ccc(C(=O)Nc2ccc(CC(=N)N)c(C(F)(F)F)c2)cc1C#Cc1cnc2cccnn12] | 3.909 |
| 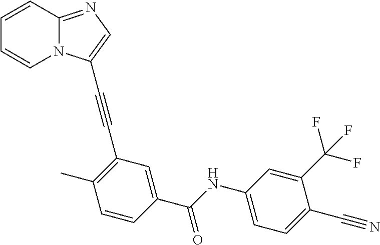 [SMILES: Cc1ccc(C(=O)Nc2ccc(C#N)c(C(F)(F)F)c2)cc1C#Cc1cnc2ccccn12] | 57.33 |
| 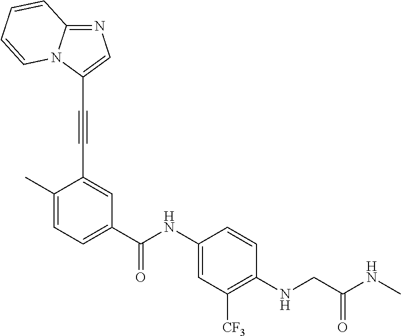 [SMILES: CNC(=O)CNc1ccc(NC(=O)c2ccc(C)c(C#Cc3cnc4ccccn34)c2)cc1C(F)(F)F] | 29.47 |
| 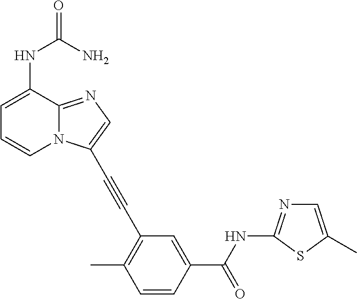 [SMILES: Cc1cnc(NC(=O)c2ccc(C)c(C#Cc3cnc4c(NC(N)=O)cccn34)c2)s1] | 2.487 |
| 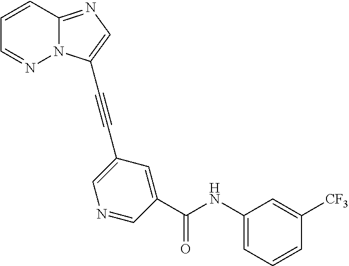 [SMILES: O=C(Nc1cccc(C(F)(F)F)c1)c1cncc(C#Cc2cnc3cccnn23)c1] | 2.066 |
| 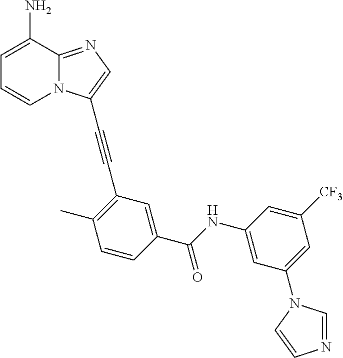 [SMILES: Cc1ccc(C(=O)Nc2cc(-n3ccnc3)cc(C(F)(F)F)c2)cc1C#Cc1cnc2c(N)cccn12] | 51.07 |
| 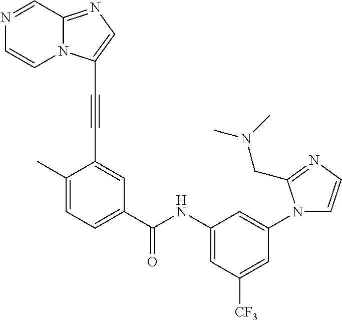 [SMILES: Cc1ccc(C(=O)Nc2cc(-n3ccnc3CN(C)C)cc(C(F)(F)F)c2)cc1C#Cc1cnc2cnccn12] | 9.219 |
| 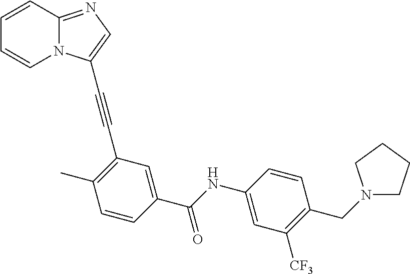 [SMILES: Cc1ccc(C(=O)Nc2ccc(CN3CCCC3)c(C(F)(F)F)c2)cc1C#Cc1cnc2ccccn12] | 60.23 |
| 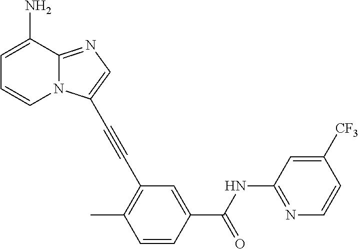 [SMILES: Cc1ccc(C(=O)Nc2cc(C(F)(F)F)ccn2)cc1C#Cc1cnc2c(N)cccn12] | 4.779 |
| 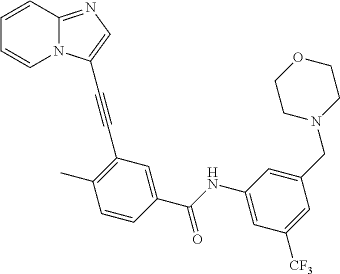 [SMILES: Cc1ccc(C(=O)Nc2cc(CN3CCOCC3)cc(C(F)(F)F)c2)cc1C#Cc1cnc2ccccn12] | 83.05 |
| 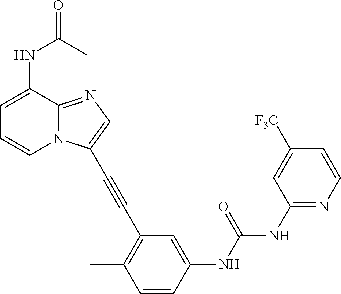 [SMILES: CC(=O)Nc1cccn2c(C#Cc3cc(NC(=O)Nc4cc(C(F)(F)F)ccn4)ccc3C)cnc12] | 5.086 |
| 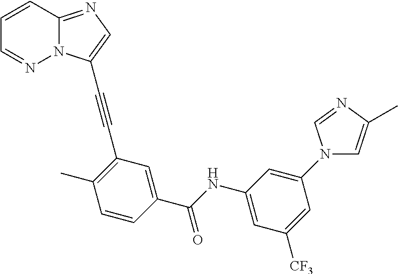 [SMILES: Cc1cn(-c2cc(NC(=O)c3ccc(C)c(C#Cc4cnc5cccnn45)c3)cc(C(F)(F)F)c2)cn1] | 21.42 |
| 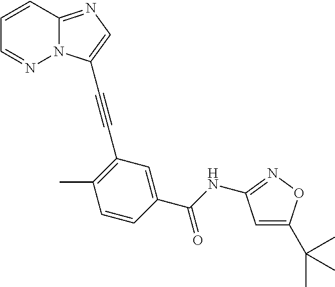 [SMILES: Cc1ccc(C(=O)Nc2cc(C(C)(C)C)on2)cc1C#Cc1cnc2cccnn12] | 4.269 |
| 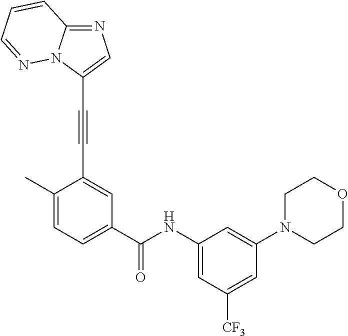 [SMILES: Cc1ccc(C(=O)Nc2cc(N3CCOCC3)cc(C(F)(F)F)c2)cc1C#Cc1cnc2cccnn12] | 17.24 |
| 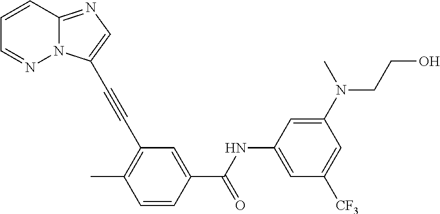 [SMILES: Cc1ccc(C(=O)Nc2cc(N(C)CCO)cc(C(F)(F)F)c2)cc1C#Cc1cnc2cccnn12] | 14.37 |
| 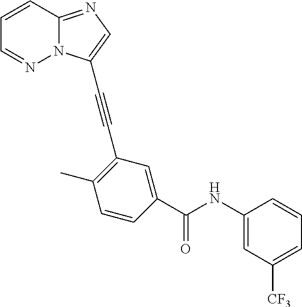 [SMILES: Cc1ccc(C(=O)Nc2cccc(C(F)(F)F)c2)cc1C#Cc1cnc2cccnn12] | 1.447 |
| 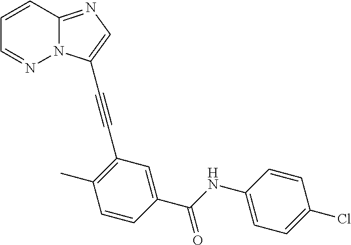 [SMILES: Cc1ccc(C(=O)Nc2ccc(Cl)cc2)cc1C#Cc1cnc2cccnn12] | 10.72 |
| 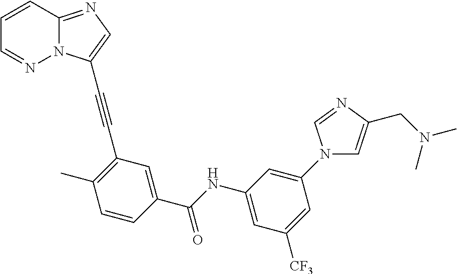 [SMILES: Cc1ccc(C(=O)Nc2cc(-n3cnc(CN(C)C)c3)cc(C(F)(F)F)c2)cc1C#Cc1cnc2cccnn12] | 3.842 |
| 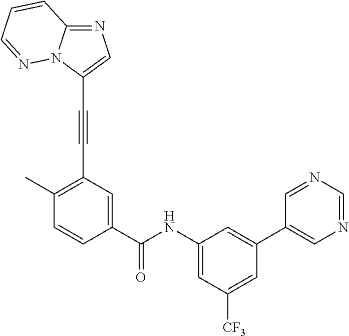 [SMILES: Cc1ccc(C(=O)Nc2cc(-c3cncnc3)cc(C(F)(F)F)c2)cc1C#Cc1cnc2cccnn12] | 8.499 |
| 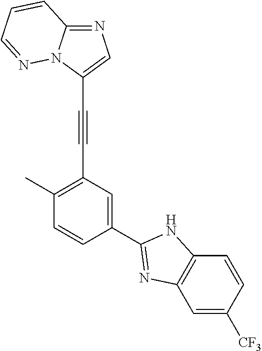 [SMILES: Cc1ccc(-c2nc3cc(C(F)(F)F)ccc3[nH]2)cc1C#Cc1cnc2cccnn12] | 5.799 |
| 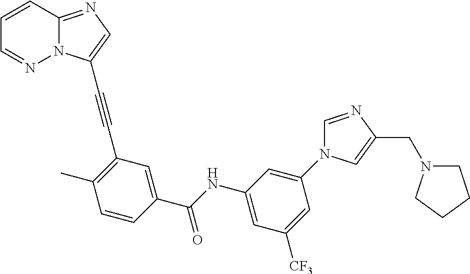 [SMILES: Cc1ccc(C(=O)Nc2cc(-n3cnc(CN4CCCC4)c3)cc(C(F)(F)F)c2)cc1C#Cc1cnc2cccnn12] | 9.193 |
| 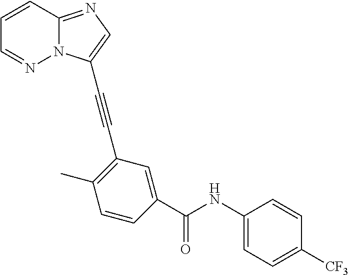 [SMILES: Cc1ccc(C(=O)Nc2ccc(C(F)(F)F)cc2)cc1C#Cc1cnc2cccnn12] | 4.392 |
| 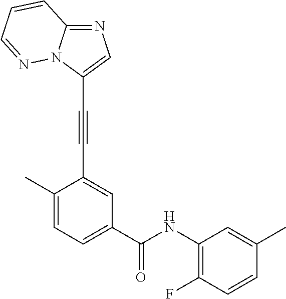 [SMILES: Cc1ccc(F)c(NC(=O)c2ccc(C)c(C#Cc3cnc4cccnn34)c2)c1] | 2.761 |
| 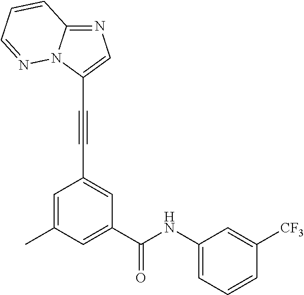 [SMILES: Cc1cc(C#Cc2cnc3cccnn23)cc(C(=O)Nc2cccc(C(F)(F)F)c2)c1] | 2943 |
| 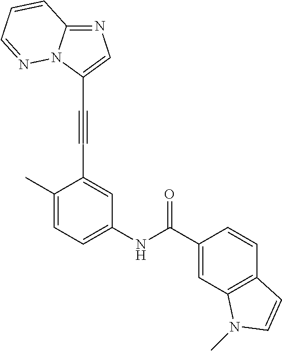 [SMILES: Cc1ccc(NC(=O)c2ccc3ccn(C)c3c2)cc1C#Cc1cnc2cccnn12] | 3.123 |
| 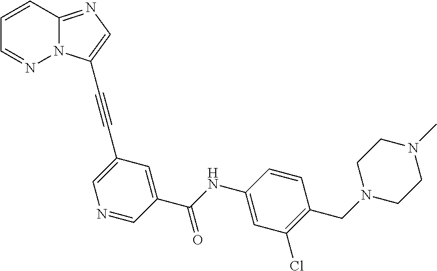 [SMILES: CN1CCN(Cc2ccc(NC(=O)c3cncc(C#Cc4cnc5cccnn45)c3)cc2Cl)CC1] | 25.61 |

The compounds can also be evaluated for their cytotoxic or growth inhibitory effects on tumor cells of interest, e.g., as described in more detail below and as shown above for some representative compounds. See e.g., WO 03/000188, pages 115-136, the full contents of which are incorporated herein by reference.

The compounds listed in Table 3 also showed inhibitory activity against certain kinases of interest.

TABLE 3
Other compounds of interest
Chemical Formula

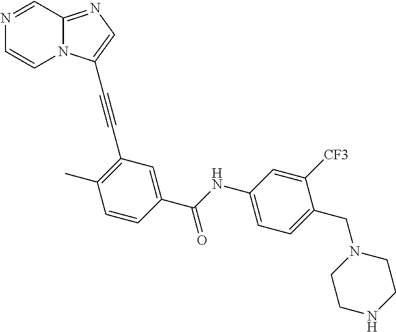 [SMILES: Cc1ccc(C(=O)Nc2ccc(CN3CCNCC3)c(C(F)(F)F)c2)cc1C#Cc1cnc2cnccn12]

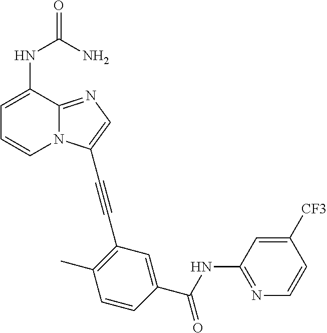 [SMILES: Cc1ccc(C(=O)Nc2cc(C(F)(F)F)ccn2)cc1C#Cc1cnc2c(NC(N)=O)cccn12]

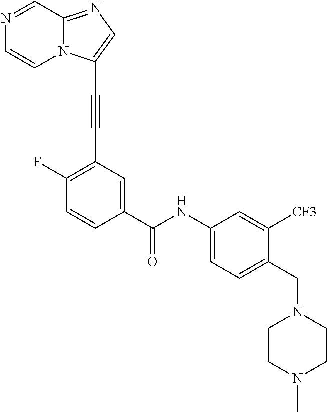 [SMILES: CN1CCN(Cc2ccc(NC(=O)c3ccc(F)c(C#Cc4cnc5cnccn45)c3)cc2C(F)(F)F)CC1]

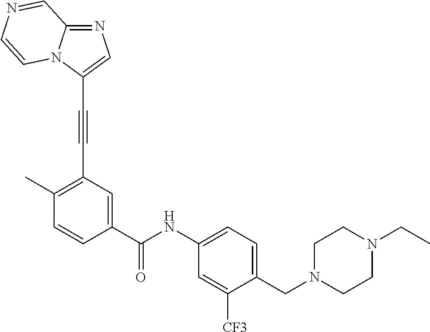 [SMILES: CCN1CCN(Cc2ccc(NC(=O)c3ccc(C)c(C#Cc4cnc5cnccn45)c3)cc2C(F)(F)F)CC1]

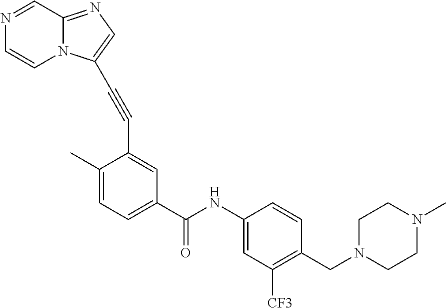 [SMILES: Cc1ccc(C(=O)Nc2ccc(CN3CCN(C)CC3)c(C(F)(F)F)c2)cc1C#Cc1cnc2cnccn12]

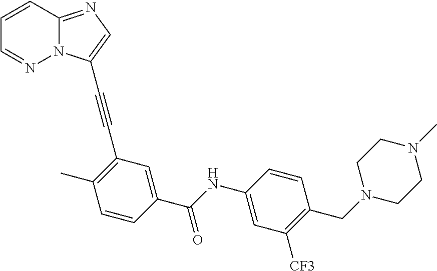 [SMILES: Cc1ccc(C(=O)Nc2ccc(CN3CCN(C)CC3)c(C(F)(F)F)c2)cc1C#Cc1cnc2cccnn12]

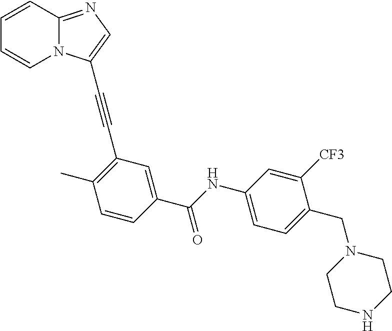 [SMILES: Cc1ccc(C(=O)Nc2ccc(CN3CCNCC3)c(C(F)(F)F)c2)cc1C#Cc1cnc2ccccn12]

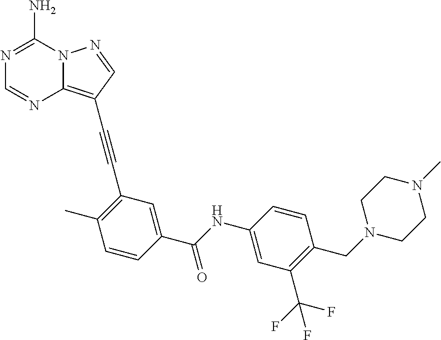 [SMILES: Cc1ccc(C(=O)Nc2ccc(CN3CCN(C)CC3)c(C(F)(F)F)c2)cc1C#Cc1cnn2c(N)ncnc12]

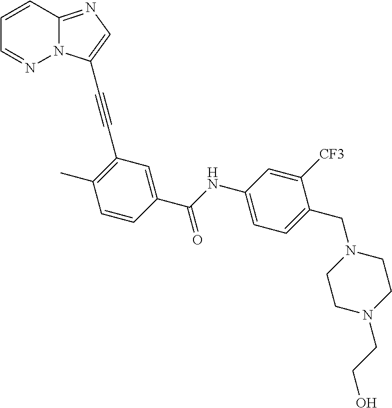 [SMILES: Cc1ccc(C(=O)Nc2ccc(CN3CCN(CCO)CC3)c(C(F)(F)F)c2)cc1C#Cc1cnc2cccnn12]

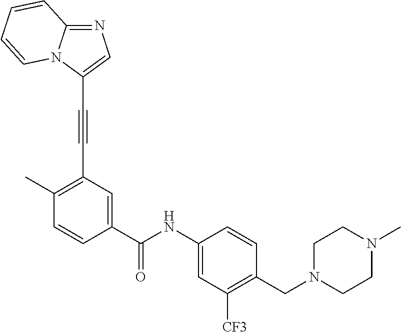 [SMILES: Cc1ccc(C(=O)Nc2ccc(CN3CCN(C)CC3)c(C(F)(F)F)c2)cc1C#Cc1cnc2ccccn12]

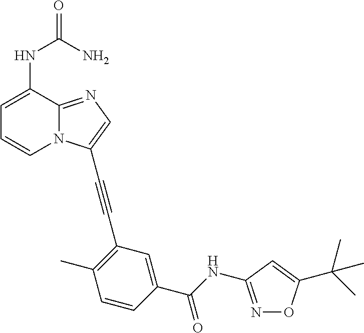 [SMILES: Cc1ccc(C(=O)Nc2cc(C(C)(C)C)on2)cc1C#Cc1cnc2c(NC(N)=O)cccn12]

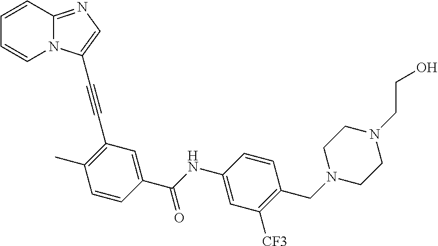 [SMILES: Cc1ccc(C(=O)Nc2ccc(CN3CCN(CCO)CC3)c(C(F)(F)F)c2)cc1C#Cc1cnc2ccccn12]

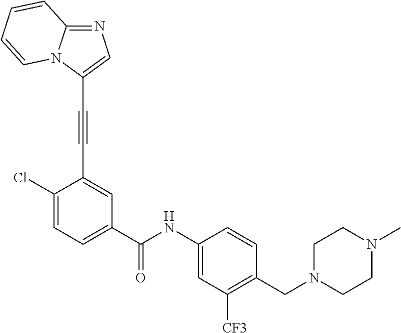 [SMILES: CN1CCN(Cc2ccc(NC(=O)c3ccc(Cl)c(C#Cc4cnc5ccccn45)c3)cc2C(F)(F)F)CC1]

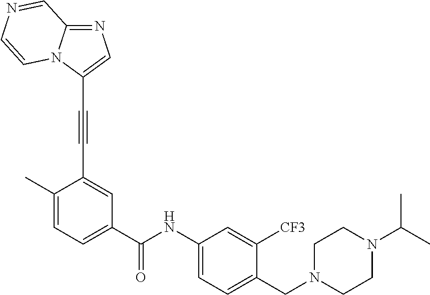 [SMILES: Cc1ccc(C(=O)Nc2ccc(CN3CCN(C(C)C)CC3)c(C(F)(F)F)c2)cc1C#Cc1cnc2cnccn12]

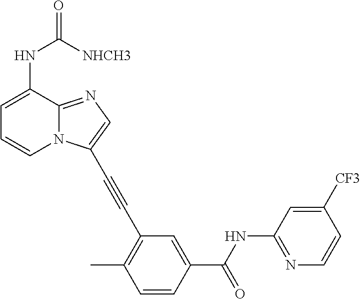 [SMILES: CNC(=O)Nc1cccn2c(C#Cc3cc(C(=O)Nc4cc(C(F)(F)F)ccn4)ccc3C)cnc12]

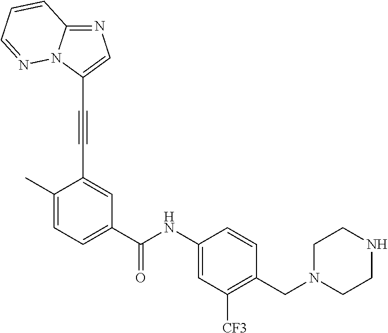 [SMILES: Cc1ccc(C(=O)Nc2ccc(CN3CCNCC3)c(C(F)(F)F)c2)cc1C#Cc1cnc2cccnn12]

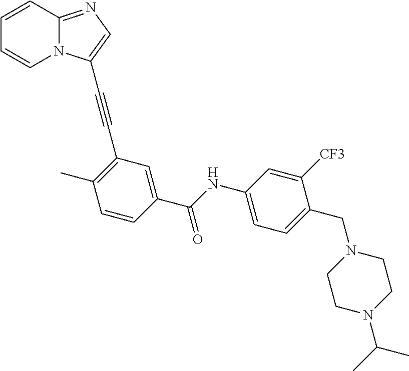 [SMILES: Cc1ccc(C(=O)Nc2ccc(CN3CCN(C(C)C)CC3)c(C(F)(F)F)c2)cc1C#Cc1cnc2ccccn12]

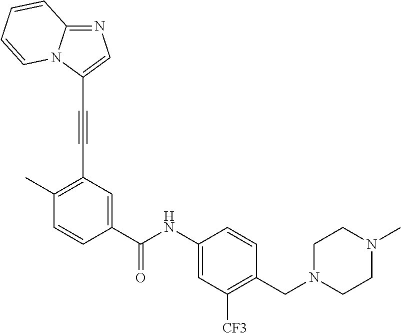 [SMILES: Cc1ccc(C(=O)Nc2ccc(CN3CCN(C)CC3)c(C(F)(F)F)c2)cc1C#Cc1cnc2ccccn12]

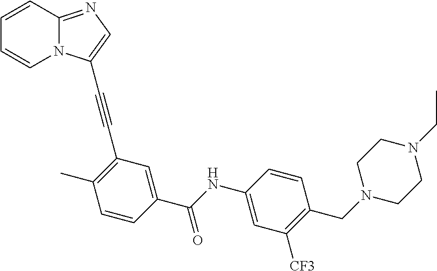 [SMILES: CCN1CCN(Cc2ccc(NC(=O)c3ccc(C)c(C#Cc4cnc5ccccn45)c3)cc2C(F)(F)F)CC1]

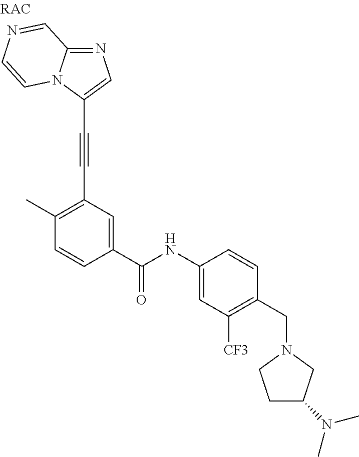 [SMILES: Cc1ccc(C(=O)Nc2ccc(CN3CC[C@@H](N(C)C)C3)c(C(F)(F)F)c2)cc1C#Cc1cnc2cnccn12]

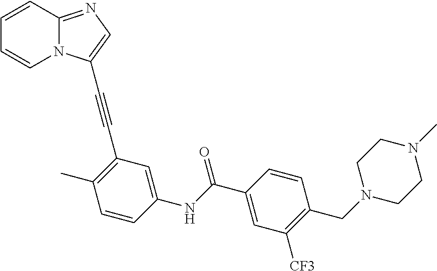 [SMILES: Cc1ccc(NC(=O)c2ccc(CN3CCN(C)CC3)c(C(F)(F)F)c2)cc1C#Cc1cnc2ccccn12]

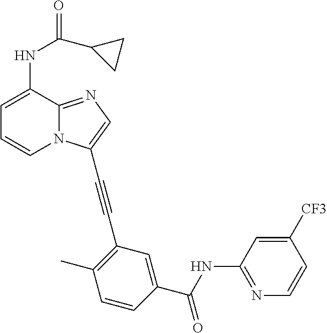 [SMILES: Cc1ccc(C(=O)Nc2cc(C(F)(F)F)ccn2)cc1C#Cc1cnc2c(NC(=O)C3CC3)cccn12]

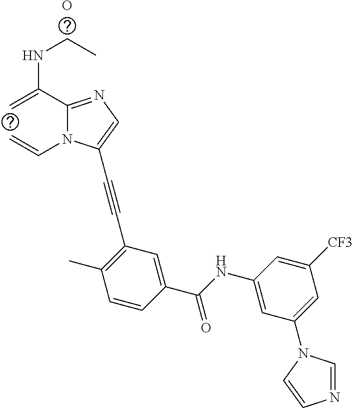

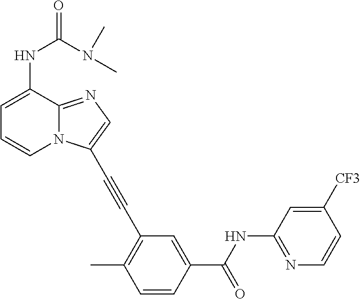 [SMILES: Cc1ccc(C(=O)Nc2cc(C(F)(F)F)ccn2)cc1C#Cc1cnc2c(NC(=O)N(C)C)cccn12]

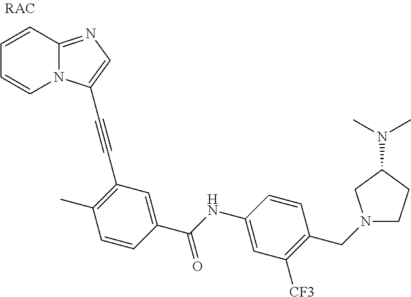 [SMILES: Cc1ccc(C(=O)Nc2ccc(CN3CC[C@@H](N(C)C)C3)c(C(F)(F)F)c2)cc1C#Cc1cnc2ccccn12]

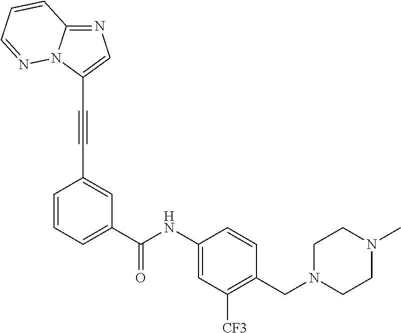 [SMILES: CN1CCN(Cc2ccc(NC(=O)c3cccc(C#Cc4cnc5cccnn45)c3)cc2C(F)(F)F)CC1]

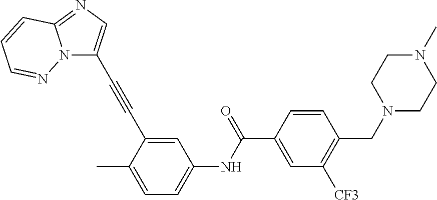 [SMILES: Cc1ccc(NC(=O)c2ccc(CN3CCN(C)CC3)c(C(F)(F)F)c2)cc1C#Cc1cnc2cccnn12]

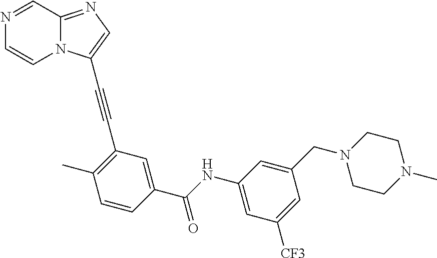 [SMILES: Cc1ccc(C(=O)Nc2cc(CN3CCN(C)CC3)cc(C(F)(F)F)c2)cc1C#Cc1cnc2cnccn12]

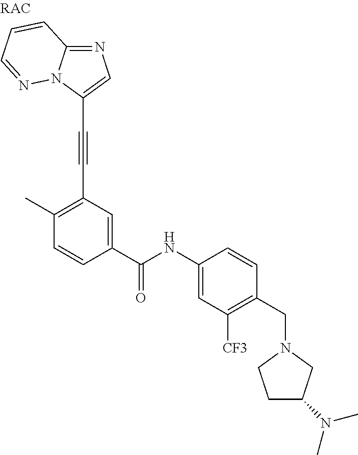 [SMILES: Cc1ccc(C(=O)Nc2ccc(CN3CC[C@@H](N(C)C)C3)c(C(F)(F)F)c2)cc1C#Cc1cnc2cccnn12]

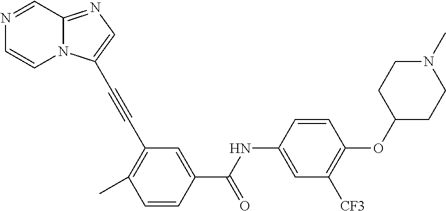 [SMILES: Cc1ccc(C(=O)Nc2ccc(OC3CCN(C)CC3)c(C(F)(F)F)c2)cc1C#Cc1cnc2cnccn12]

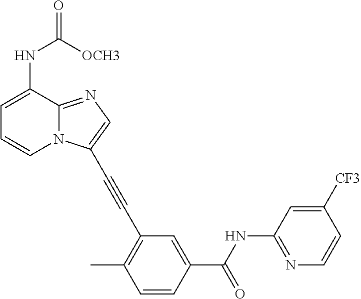 [SMILES: COC(=O)Nc1cccn2c(C#Cc3cc(C(=O)Nc4cc(C(F)(F)F)ccn4)ccc3C)cnc12]

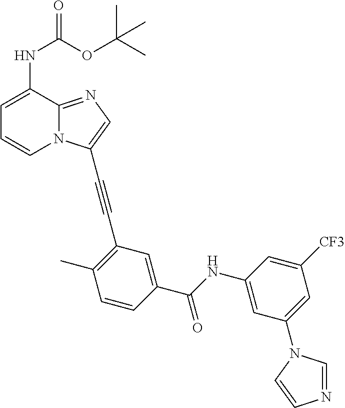 [SMILES: Cc1ccc(C(=O)Nc2cc(-n3ccnc3)cc(C(F)(F)F)c2)cc1C#Cc1cnc2c(NC(=O)OC(C)(C)C)cccn12]

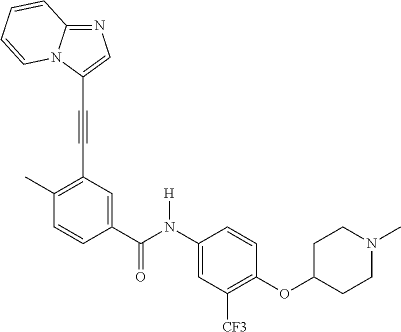 [SMILES: Cc1ccc(C(=O)Nc2ccc(OC3CCN(C)CC3)c(C(F)(F)F)c2)cc1C#Cc1cnc2ccccn12]

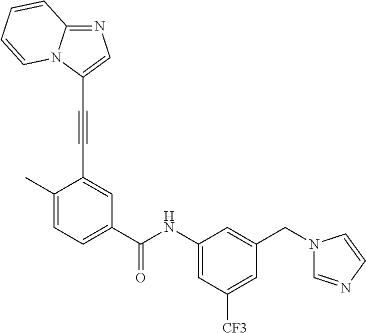 [SMILES: Cc1ccc(C(=O)Nc2cc(Cn3ccnc3)cc(C(F)(F)F)c2)cc1C#Cc1cnc2ccccn12]

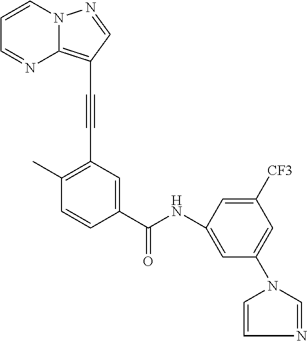 [SMILES: Cc1ccc(C(=O)Nc2cc(-n3ccnc3)cc(C(F)(F)F)c2)cc1C#Cc1cnn2cccnc12]

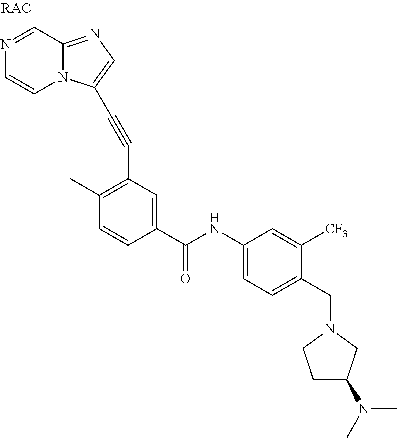 [SMILES: Cc1ccc(C(=O)Nc2ccc(CN3CC[C@H](N(C)C)C3)c(C(F)(F)F)c2)cc1C#Cc1cnc2cnccn12]

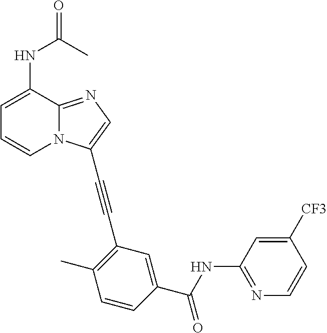 [SMILES: CC(=O)Nc1cccn2c(C#Cc3cc(C(=O)Nc4cc(C(F)(F)F)ccn4)ccc3C)cnc12]

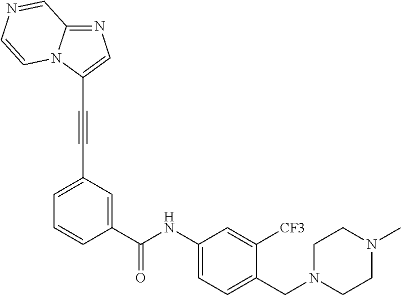 [SMILES: CN1CCN(Cc2ccc(NC(=O)c3cccc(C#Cc4cnc5cnccn45)c3)cc2C(F)(F)F)CC1]

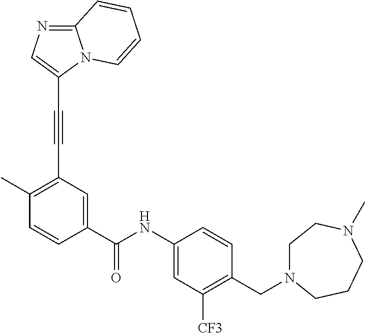

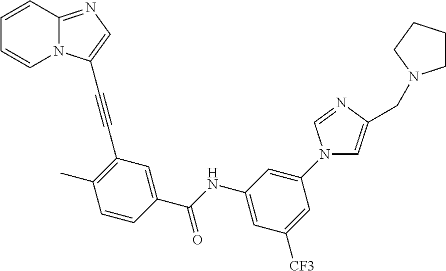 [SMILES: Cc1ccc(C(=O)Nc2cc(-n3cnc(CN4CCCC4)c3)cc(C(F)(F)F)c2)cc1C#Cc1cnc2ccccn12]

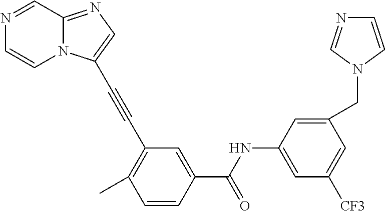 [SMILES: Cc1ccc(C(=O)Nc2cc(Cn3ccnc3)cc(C(F)(F)F)c2)cc1C#Cc1cnc2cnccn12]

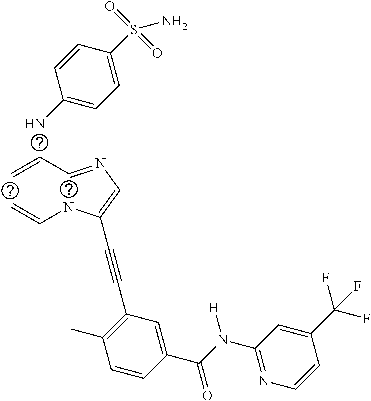

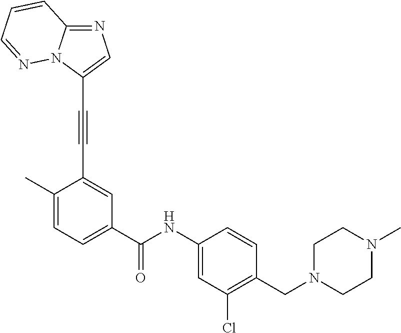 [SMILES: Cc1ccc(C(=O)Nc2ccc(CN3CCN(C)CC3)c(Cl)c2)cc1C#Cc1cnc2cccnn12]

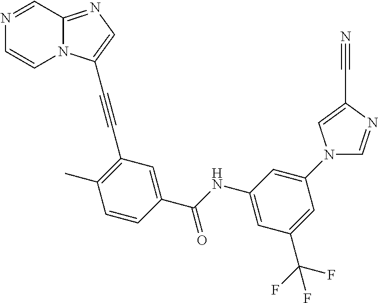 [SMILES: Cc1ccc(C(=O)Nc2cc(-n3cnc(C#N)c3)cc(C(F)(F)F)c2)cc1C#Cc1cnc2cnccn12]

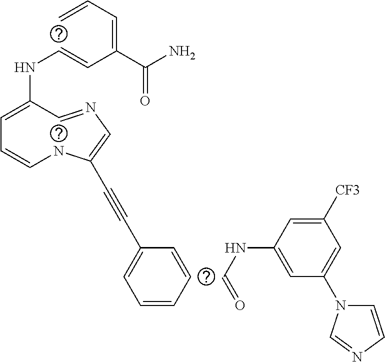

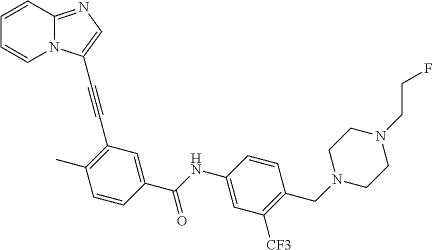 [SMILES: Cc1ccc(C(=O)Nc2ccc(CN3CCN(CCF)CC3)c(C(F)(F)F)c2)cc1C#Cc1cnc2ccccn12]

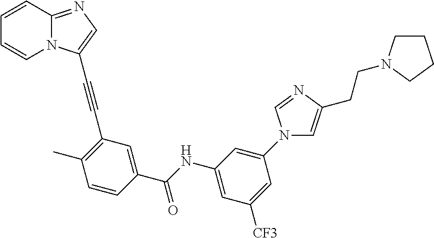 [SMILES: Cc1ccc(C(=O)Nc2cc(-n3cnc(CCN4CCCC4)c3)cc(C(F)(F)F)c2)cc1C#Cc1cnc2ccccn12]

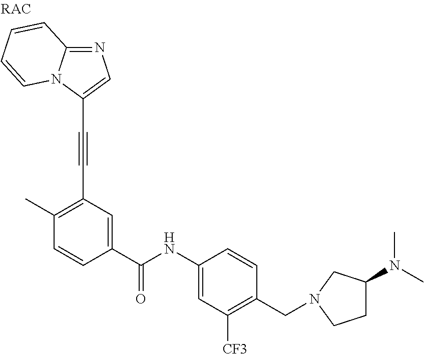 [SMILES: Cc1ccc(C(=O)Nc2ccc(CN3CC[C@H](N(C)C)C3)c(C(F)(F)F)c2)cc1C#Cc1cnc2ccccn12]

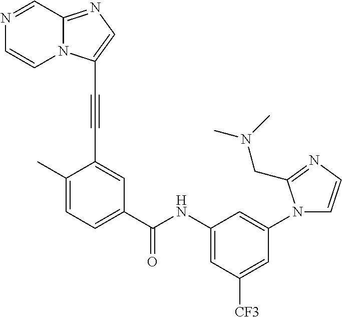 [SMILES: Cc1ccc(C(=O)Nc2cc(-n3ccnc3CN(C)C)cc(C(F)(F)F)c2)cc1C#Cc1cnc2cnccn12]

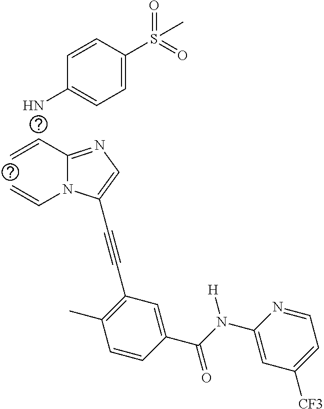

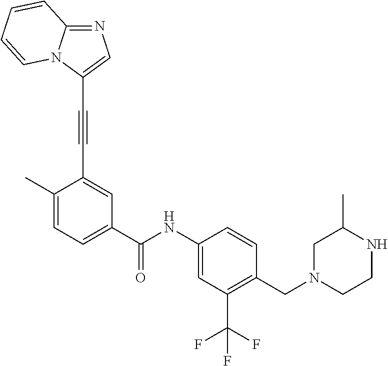 [SMILES: Cc1ccc(C(=O)Nc2ccc(CN3CCNC(C)C3)c(C(F)(F)F)c2)cc1C#Cc1cnc2ccccn12]

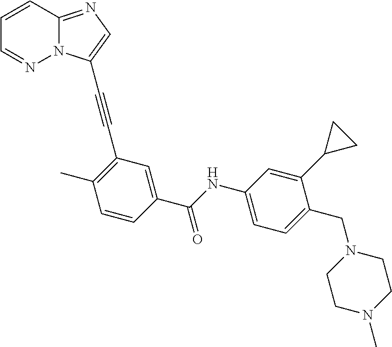 [SMILES: Cc1ccc(C(=O)Nc2ccc(CN3CCN(C)CC3)c(C3CC3)c2)cc1C#Cc1cnc2cccnn12]

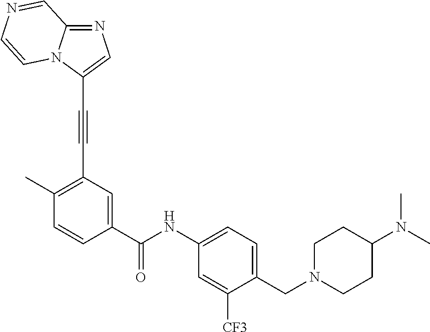 [SMILES: Cc1ccc(C(=O)Nc2ccc(CN3CCC(N(C)C)CC3)c(C(F)(F)F)c2)cc1C#Cc1cnc2cnccn12]

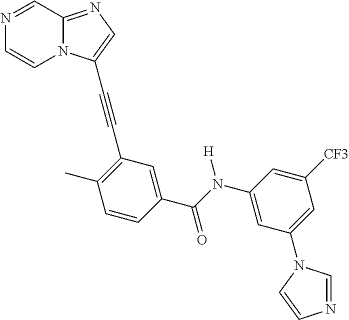 [SMILES: Cc1ccc(C(=O)Nc2cc(-n3ccnc3)cc(C(F)(F)F)c2)cc1C#Cc1cnc2cnccn12]

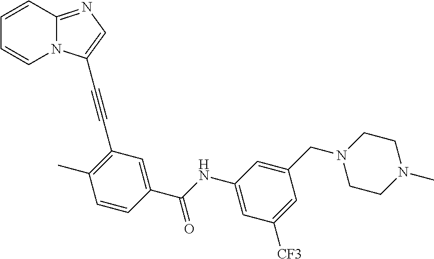 [SMILES: Cc1ccc(C(=O)Nc2cc(CN3CCN(C)CC3)cc(C(F)(F)F)c2)cc1C#Cc1cnc2ccccn12]

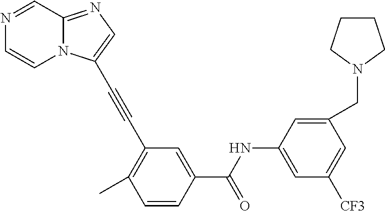 [SMILES: Cc1ccc(C(=O)Nc2cc(CN3CCCC3)cc(C(F)(F)F)c2)cc1C#Cc1cnc2cnccn12]

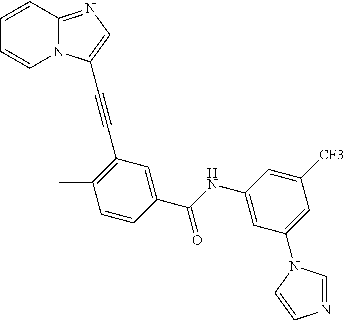 [SMILES: Cc1ccc(C(=O)Nc2cc(-n3ccnc3)cc(C(F)(F)F)c2)cc1C#Cc1cnc2ccccn12]

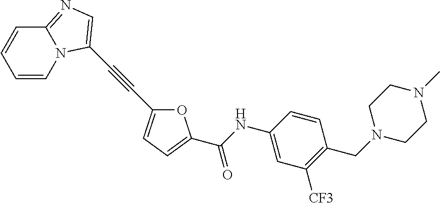 [SMILES: CN1CCN(Cc2ccc(NC(=O)c3ccc(C#Cc4cnc5ccccn45)o3)cc2C(F)(F)F)CC1]

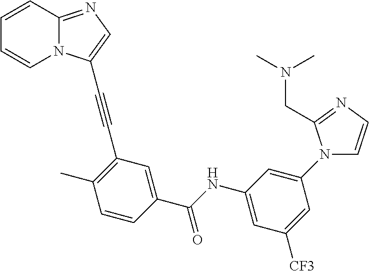 [SMILES: Cc1ccc(C(=O)Nc2cc(-n3ccnc3CN(C)C)cc(C(F)(F)F)c2)cc1C#Cc1cnc2ccccn12]

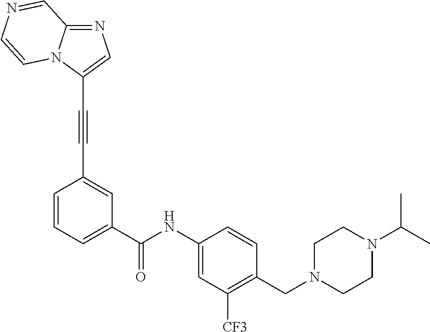 [SMILES: CC(C)N1CCN(Cc2ccc(NC(=O)c3cccc(C#Cc4cnc5cnccn45)c3)cc2C(F)(F)F)CC1]

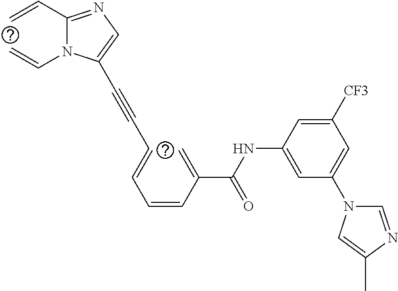 [SMILES: C=Cc1ncc(C#C/C=C/C=C\C(=C)C(=O)Nc2cc(-n3cnc(C)c3)cc(C(F)(F)F)c2)n1C=C]

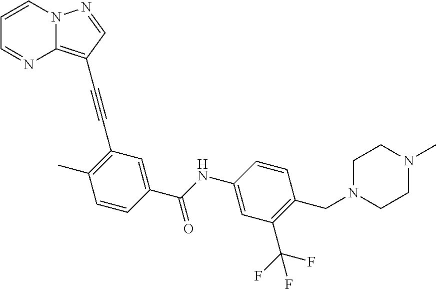 [SMILES: Cc1ccc(C(=O)Nc2ccc(CN3CCN(C)CC3)c(C(F)(F)F)c2)cc1C#Cc1cnn2cccnc12]

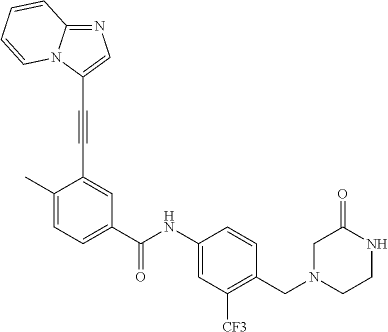 [SMILES: Cc1ccc(C(=O)Nc2ccc(CN3CCNC(=O)C3)c(C(F)(F)F)c2)cc1C#Cc1cnc2ccccn12]

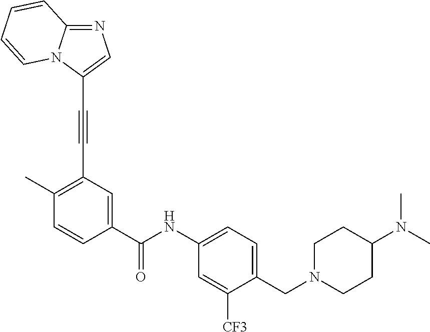 [SMILES: Cc1ccc(C(=O)Nc2ccc(CN3CCC(N(C)C)CC3)c(C(F)(F)F)c2)cc1C#Cc1cnc2ccccn12]

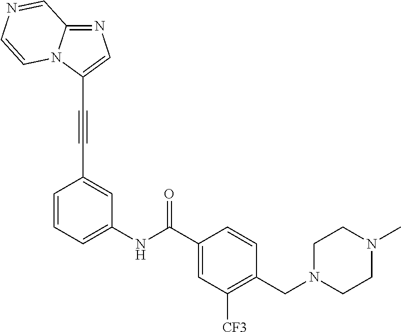 [SMILES: CN1CCN(Cc2ccc(C(=O)Nc3cccc(C#Cc4cnc5cnccn45)c3)cc2C(F)(F)F)CC1]

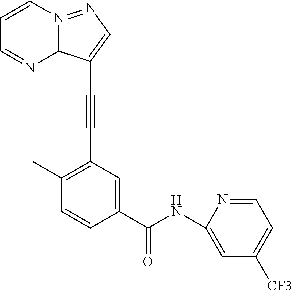

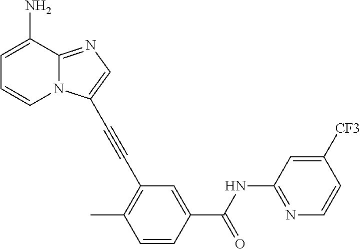 [SMILES: Cc1ccc(C(=O)Nc2cc(C(F)(F)F)ccn2)cc1C#Cc1cnc2c(N)cccn12]

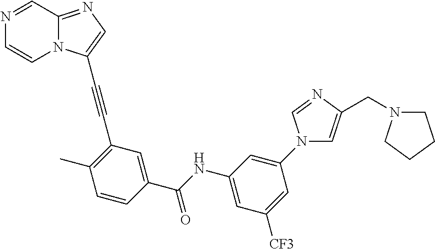 [SMILES: Cc1ccc(C(=O)Nc2cc(-n3cnc(CN4CCCC4)c3)cc(C(F)(F)F)c2)cc1C#Cc1cnc2cnccn12]

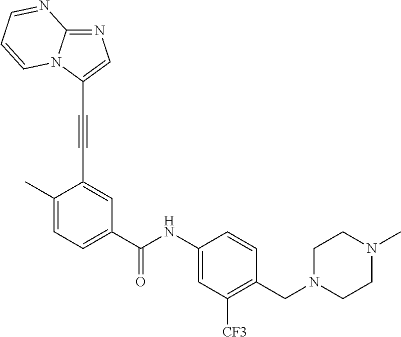 [SMILES: Cc1ccc(C(=O)Nc2ccc(CN3CCN(C)CC3)c(C(F)(F)F)c2)cc1C#Cc1cnc2ncccn12]

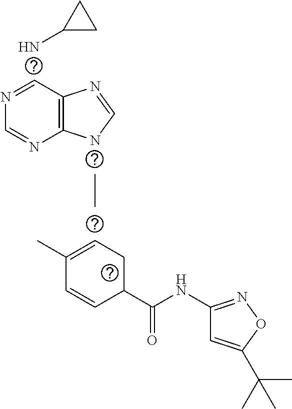

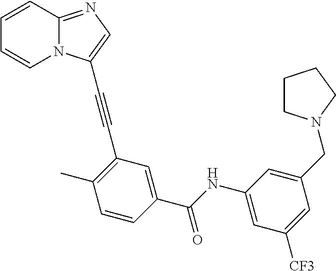 [SMILES: Cc1ccc(C(=O)Nc2cc(CN3CCCC3)cc(C(F)(F)F)c2)cc1C#Cc1cnc2ccccn12]

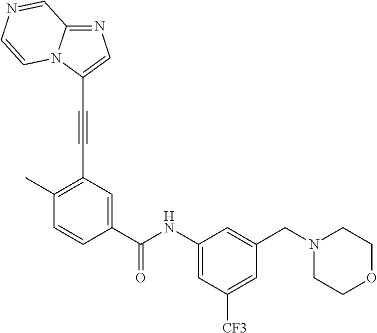 [SMILES: Cc1ccc(C(=O)Nc2cc(CN3CCOCC3)cc(C(F)(F)F)c2)cc1C#Cc1cnc2cnccn12]

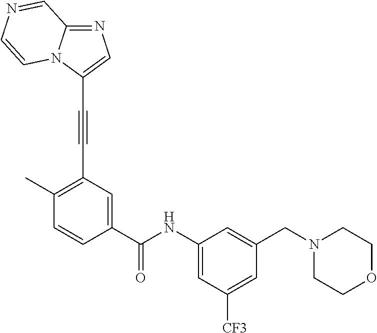 [SMILES: Cc1ccc(C(=O)Nc2cc(CN3CCOCC3)cc(C(F)(F)F)c2)cc1C#Cc1cnc2cnccn12]

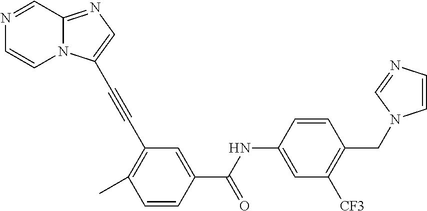 [SMILES: Cc1ccc(C(=O)Nc2ccc(Cn3ccnc3)c(C(F)(F)F)c2)cc1C#Cc1cnc2cnccn12]

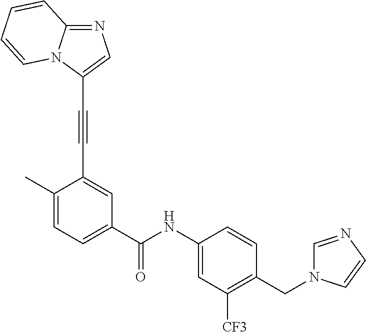 [SMILES: Cc1ccc(C(=O)Nc2ccc(Cn3ccnc3)c(C(F)(F)F)c2)cc1C#Cc1cnc2ccccn12]

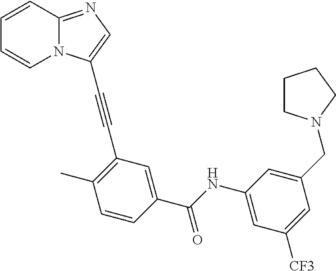 [SMILES: Cc1ccc(C(=O)Nc2cc(CN3CCCC3)cc(C(F)(F)F)c2)cc1C#Cc1cnc2ccccn12]

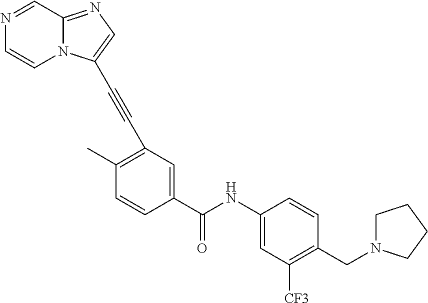 [SMILES: Cc1ccc(C(=O)Nc2ccc(CN3CCCC3)c(C(F)(F)F)c2)cc1C#Cc1cnc2cnccn12]

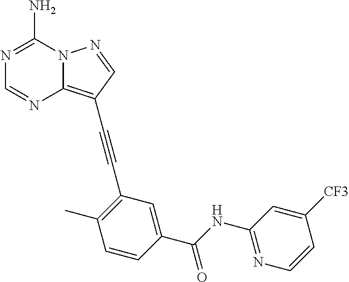 [SMILES: Cc1ccc(C(=O)Nc2cc(C(F)(F)F)ccn2)cc1C#Cc1cnn2c(N)ncnc12]

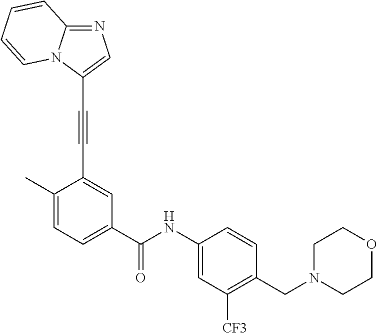 [SMILES: Cc1ccc(C(=O)Nc2ccc(CN3CCOCC3)c(C(F)(F)F)c2)cc1C#Cc1cnc2ccccn12]

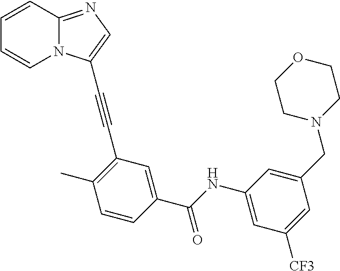 [SMILES: Cc1ccc(C(=O)Nc2cc(CN3CCOCC3)cc(C(F)(F)F)c2)cc1C#Cc1cnc2ccccn12]

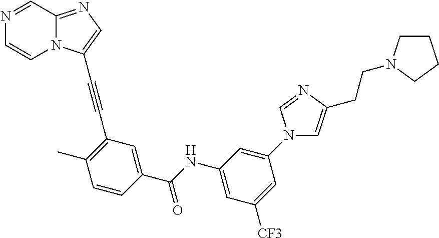 [SMILES: Cc1ccc(C(=O)Nc2cc(-n3cnc(CCN4CCCC4)c3)cc(C(F)(F)F)c2)cc1C#Cc1cnc2cnccn12]

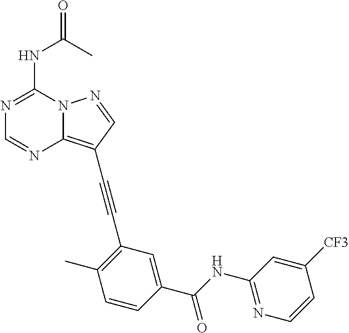 [SMILES: CC(=O)Nc1ncnc2c(C#Cc3cc(C(=O)Nc4cc(C(F)(F)F)ccn4)ccc3C)cnn12]

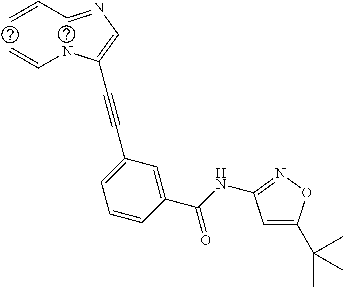 [SMILES: C=C/C=N/C=C(/C#Cc1cccc(C(=O)Nc2cc(C(C)(C)C)on2)c1)NC=C]

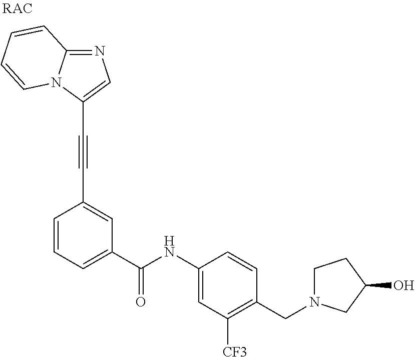 [SMILES: O=C(Nc1ccc(CN2CC[C@@H](O)C2)c(C(F)(F)F)c1)c1cccc(C#Cc2cnc3ccccn23)c1]

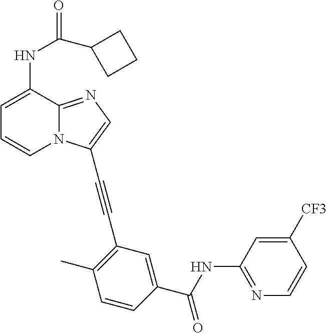 [SMILES: Cc1ccc(C(=O)Nc2cc(C(F)(F)F)ccn2)cc1C#Cc1cnc2c(NC(=O)C3CCC3)cccn12]

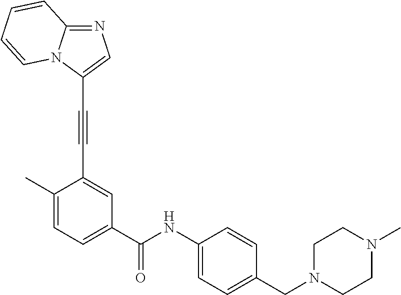 [SMILES: Cc1ccc(C(=O)Nc2ccc(CN3CCN(C)CC3)cc2)cc1C#Cc1cnc2ccccn12]

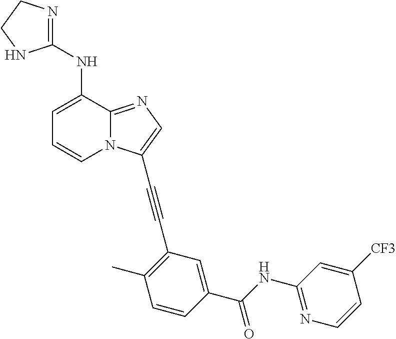 [SMILES: Cc1ccc(C(=O)Nc2cc(C(F)(F)F)ccn2)cc1C#Cc1cnc2c(NC3=NCCN3)cccn12]

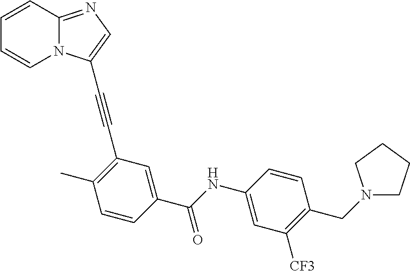 [SMILES: Cc1ccc(C(=O)Nc2ccc(CN3CCCC3)c(C(F)(F)F)c2)cc1C#Cc1cnc2ccccn12]

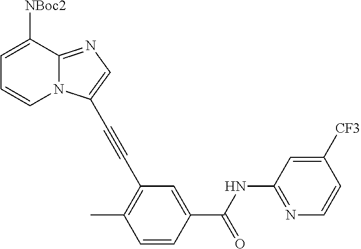

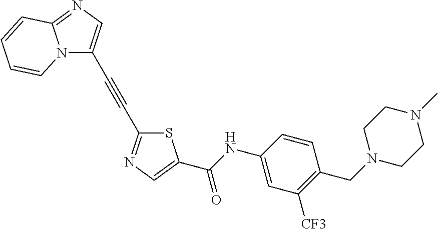 [SMILES: CN1CCN(Cc2ccc(NC(=O)c3cnc(C#Cc4cnc5ccccn45)s3)cc2C(F)(F)F)CC1]

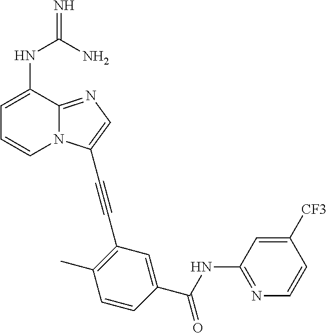 [SMILES: Cc1ccc(C(=O)Nc2cc(C(F)(F)F)ccn2)cc1C#Cc1cnc2c(NC(=N)N)cccn12]

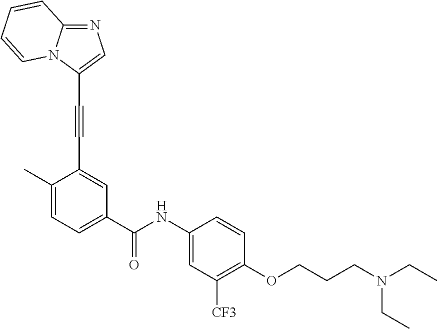 [SMILES: CCN(CC)CCCOc1ccc(NC(=O)c2ccc(C)c(C#Cc3cnc4ccccn34)c2)cc1C(F)(F)F]

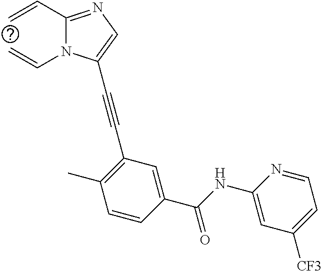 [SMILES: C=Cc1ncc(C#Cc2cc(C(=O)Nc3cc(C(F)(F)F)ccn3)ccc2C)n1C=C]

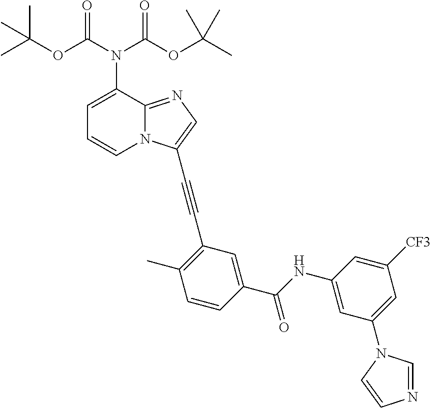 [SMILES: Cc1ccc(C(=O)Nc2cc(-n3ccnc3)cc(C(F)(F)F)c2)cc1C#Cc1cnc2c(N(C(=O)OC(C)(C)C)C(=O)OC(C)(C)C)cccn12]

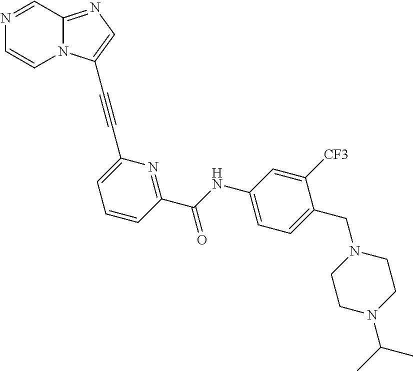 [SMILES: CC(C)N1CCN(Cc2ccc(NC(=O)c3cccc(C#Cc4cnc5cnccn45)n3)cc2C(F)(F)F)CC1]

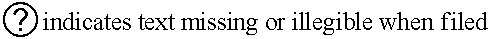

Using the methodology of the kinase assay described above, ponatinib was determined by the assays to inhibit the kinase activity of all three RAF tyrosine kinases, as more specifically described in Table 4:

TABLE 4
Ponatinib RAF Activity

| Target Kinase | IC50 (nm) |
|---|---|
| A-RAF | 71 |
| B-RAF | 33 |
| C-RAF | 17 |

Cell Growth Assay:

Cells were treated with ponatinib or vehicle (DMSO) for 72 hours. Cell growth was assessed using the Cell Titer 96 Aqueous One Solution Cell Proliferation Assay (Promega) and absorbance was measured using a Wallac Victor microplate reader (PerkinElmer). To differentiate between a cytostatic and cytotoxic drug effect, the concentration that causes 50% growth inhibition (GI50) was determined by correcting for the cell count at time zero (time of treatment) and plotting data as percent growth relative to vehicle-treated cells using XLfit version 5.2.0 for Microsoft Excel. Data are shown as mean from 3 independent experiments performed in triplicate.

Cell growth assays were also performed as part of an automated cell line screening assay (Ricerca Biosciences, LLC, Bothell, Wash., USA). Experimental procedures are similar to those described above.

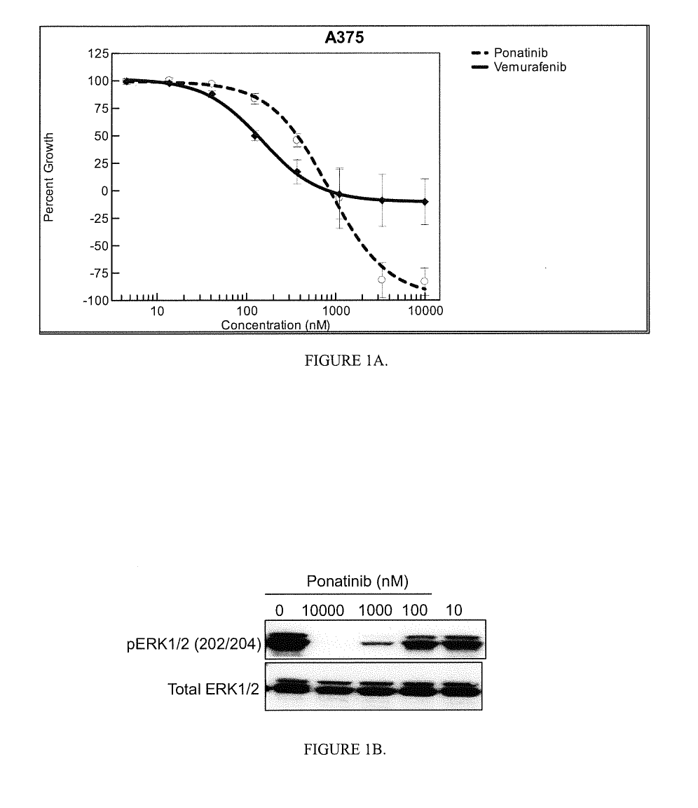

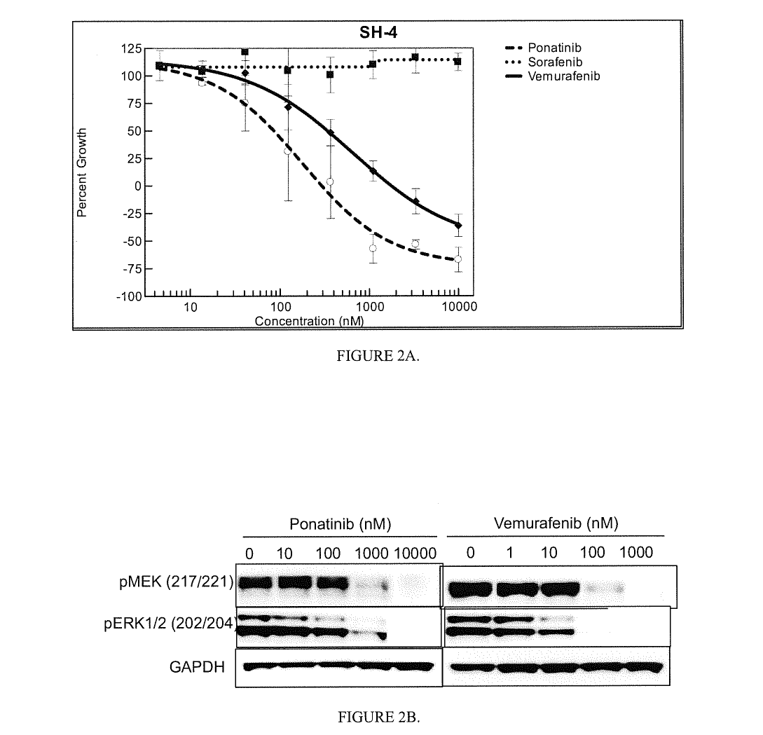

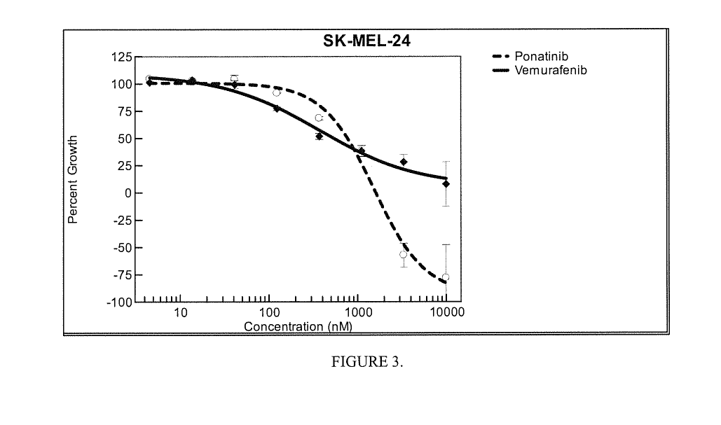

Ponatinib was determined by the assays to inhibit the growth of BRAF^{V600E} mutant melanoma and colorectal cancer cell lines, as more specifically described in FIGS. 1-4 and Table 5:

TABLE 5
Ponatinib BRAF^{V600E} Mutant Activity

| Cancer | Oncogene | Cell line | Ponatinib GI50 (nM) |
|---|---|---|---|
| Melanoma | BRAF V600E | A375 | 372 |
| Melanoma | BRAF V600E | SH-4 | 85.6 |
| Melanoma | BRAF V600E | SK-MEL-24 | 767 |
| Colorectal | BRAF V600E | HT-29 | 354 |
| Melanoma | BRAF wt | CHL-1* | 88.5 |
| Melanoma | BRAF V600E het | C32* | 107 |

*Cell lines tested under the direction of applicants by Ricerca Biosciences.

Immunoblot analysis: To examine inhibition of BRAF signaling, cells were treated with compound or vehicle (DMSO) over a range of concentrations for 3 hours. Cells were lysed in SDS lysis buffer (0.06 M Tris-HCL. 1% SDS and 10% glycerol) and protein concentration was determined using a BCA Protein assay (Thermo Scientific). Cellular lysates (50 n) were resolved by electrophoresis and transferred to nitrocellulose membranes using NuPage Novex reagents (Invitrogen). Membranes were immunoblotted with the indicated antibodies (Cell Signaling Technology) and then exposed to Supersignal ELISA femto maximum sensitivity substrate (Thermo Scientific) to generate a chemiluminescent signal.

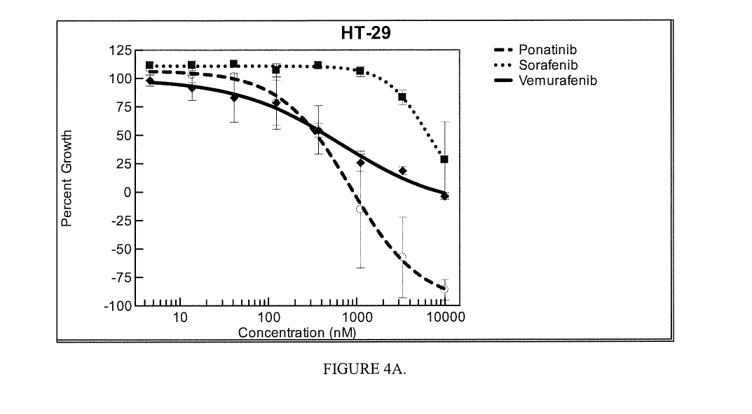

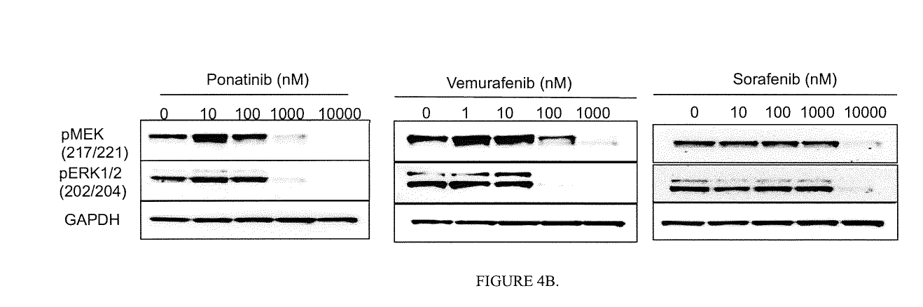

Ponatinib was determined by the assays to inhibit MEK or ERK1/2 phosphorylation, downstream targets of activated BRAF, in A375 and SH-4 BRAF^{V600E} mutant melanoma cancer cells (FIGS. 1B and 2B) and in HT-29 BRAF^{V600E} mutant colorectal cancer cells (FIG. 4B).

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

What is claimed is:

1. A method for treating or preventing a RAF kinase mediated disease or condition in a subject in need thereof comprising administering to the subject an effective amount of a RAF inhibitor, wherein the RAF inhibitor is a compound of Formula I: 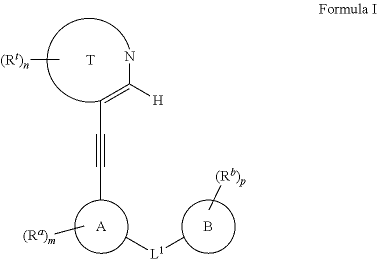
or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;
or a pharmaceutically acceptable salt, solvate or hydrate thereof.

2. A method according to claim 1, wherein in the compound of Formula I, Ring T is: 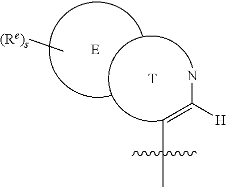
wherein Ring E is a 5- or 6-membered unsaturated ring comprising 0-3 heteroatoms selected from O, N, and S, and s is 0, 1, 2, 3 or 4.

3. A method according to claim 1, wherein in the compound of Formula I, Ring T is a bicyclic heteroaryl ring selected from: 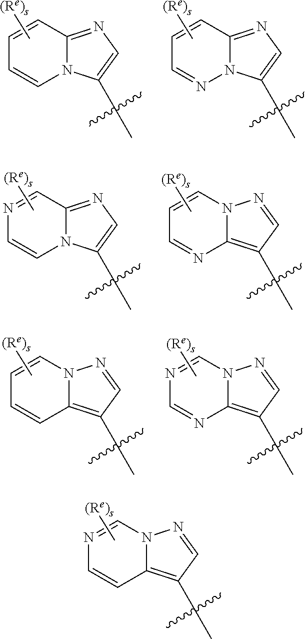
and s is 0, 1, 2, 3 or 4.

4. A method according to claim 1, wherein the RAF inhibitor is a compound of Formula II: 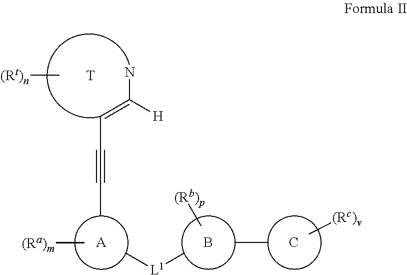
wherein:
Ring C is a 5- or 6-membered heterocyclic or heteroaryl ring, comprising carbon atoms and 1-3 heteroatoms independently selected from O, N and S(O)ᵣ;
R^{c}, at each occurrence, is independently selected from halo, ═O, —CN, —NO₂, —R⁴, —OR², —NR²R³, —C(O)YR², —OC(O)YR², —NR²C(O)YR², —Si(R²)₃, —SC(O)YR², —NR²C(═S)YR², —OC(═S)YR², —C(═S)YR², —YC(═NR³)YR², —YP(═O)(YR⁴)(YR⁴), —NR²SO₂R², —S(O)ᵣR², —SO₂NR²R³ and —NR²SO₂NR²R³, wherein each Y is independently a bond, —O—, —S— or —NR³—; and,
v is 0, 1, 2, 3, 4 or 5.

5. A method according to claim 4, wherein Ring T is: 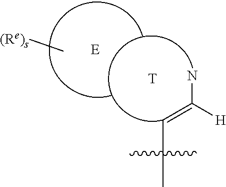
wherein Ring E is a 5- or 6-membered unsaturated ring comprising 0-3 heteroatoms selected from O, N, and S, and s is 0, 1, 2, 3 or 4.

6. A method according to claim 5, wherein Rings A and B are aryl.

7. A method according to claim 5, wherein Ring C is imidazolyl.

8. A method according to claim 7, wherein the RAF inhibitor is a compound selected from Formulae IIa, IIb, or IIc:

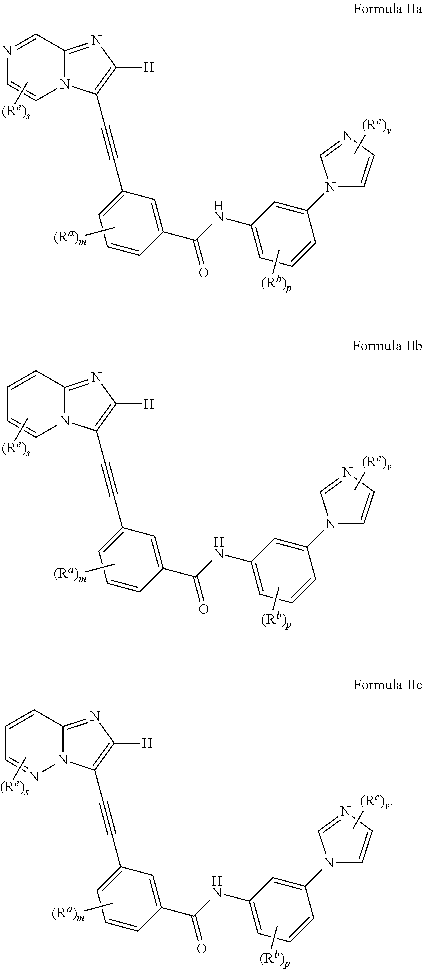

9. A method according to claim 8, wherein s is 0; m, p and v are 1; R^{a} and R^{c} are methyl; and R^{b} is CF₃.

10. A method according to claim 1, wherein the RAF inhibitor is a compound of Formula III: 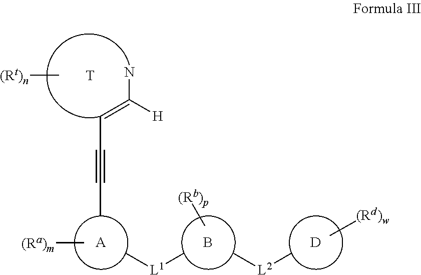
wherein:
Ring D represents a 5-, 6-heterocyclic or heteroaryl ring comprising carbon atoms and 1-3 heteroatoms independently selected from O, N and S(O)ᵣ;
L² is (CH₂)_{z}, O(CH₂)ₓ, NR³(CH₂)ₓ, S(CH₂)ₓ or (CH₂)ₓNR³C(O)(CH₂)ₓ in either direction;
R^{d}, at each occurrence, is selected from the group consisting of H, halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
p is 0, 1, 2, 3 or 4;
w is 0, 1, 2, 3, 4 or 5;
x is 0, 1, 2 or 3; and,
z is 1, 2, 3 or 4.

11. A method according to claim 10, wherein Ring T has the following structure: 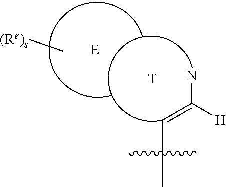
wherein Ring E is a 5- or 6-membered unsaturated ring comprising 0-3 heteroatoms selected from O, N, and S, and s is 0, 1, 2, 3 or 4.

12. A method according to claim 11, wherein Rings A and B are aryl.

13. A method according to claim 11, wherein Ring T is a bicyclic heteroaryl ring selected from: 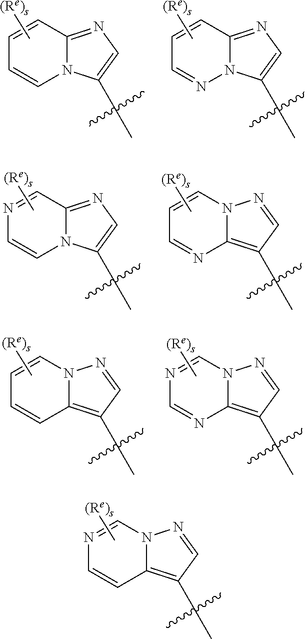
and s is 0, 1, 2, 3 or 4.

14. A method according to claim 13, wherein Ring D is piperazinyl and L² is CH₂.

15. A method according to claim 14 wherein the RAF inhibitor is a compound selected from Formulae IIIa, IIIb, and IIIc:

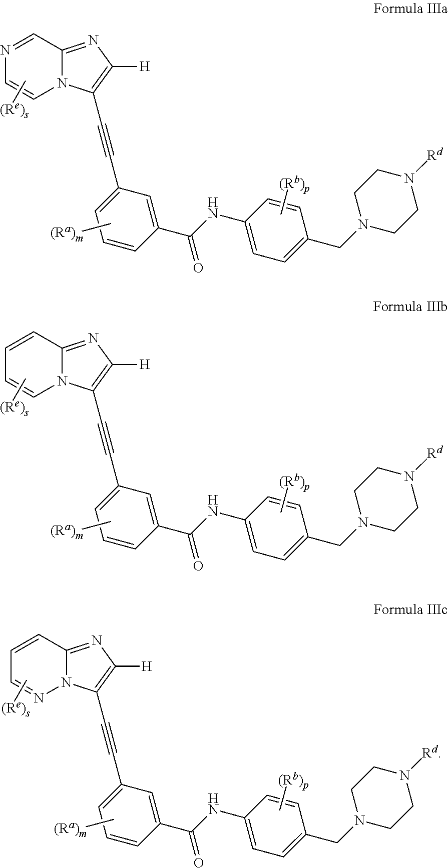

16. A method according to claim 15 wherein s is 0, m is 1, p is 1, R^{a} is methyl, R^{b} is CF₃, and R^{d} is methyl or —CH₂CH₂OH.

17. A method according to claim 1, wherein the RAF inhibitor is a compound selected from: 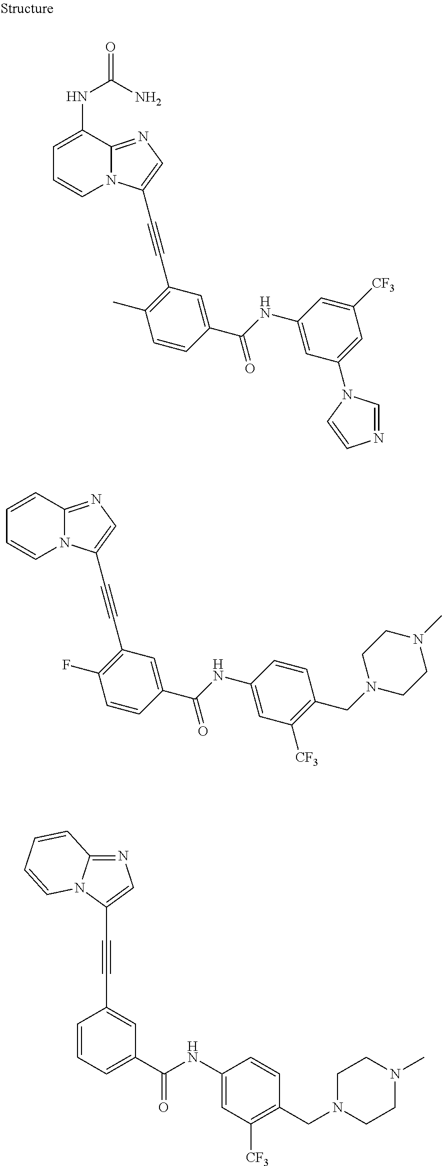 [SMILES: Cc1ccc(C(=O)Nc2cc(-n3ccnc3)cc(C(F)(F)F)c2)cc1C#Cc1cnc2c(NC(N)=O)cccn12 ; CN1CCN(Cc2ccc(NC(=O)c3ccc(F)c(C#Cc4cnc5ccccn45)c3)cc2C(F)(F)F)CC1 ; CN1CCN(Cc2ccc(NC(=O)c3cccc(C#Cc4cnc5ccccn45)c3)cc2C(F)(F)F)CC1] 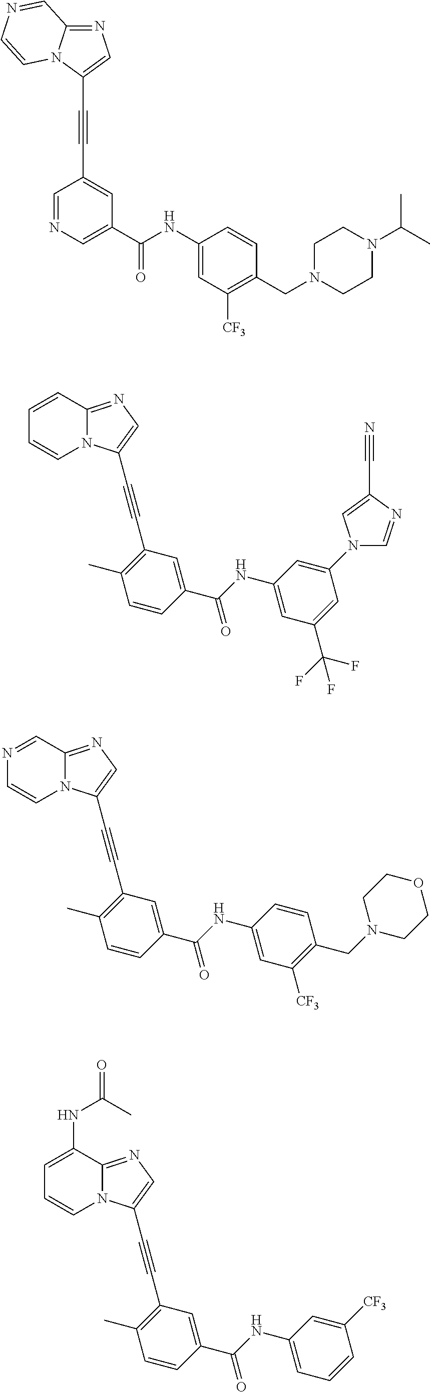 [SMILES: CC(C)N1CCN(Cc2ccc(NC(=O)c3cncc(C#Cc4cnc5cnccn45)c3)cc2C(F)(F)F)CC1 ; Cc1ccc(C(=O)Nc2cc(-n3cnc(C#N)c3)cc(C(F)(F)F)c2)cc1C#Cc1cnc2ccccn12 ; Cc1ccc(C(=O)Nc2ccc(CN3CCOCC3)c(C(F)(F)F)c2)cc1C#Cc1cnc2cnccn12 ; CC(=O)Nc1cccn2c(C#Cc3cc(C(=O)Nc4cccc(C(F)(F)F)c4)ccc3C)cnc12] 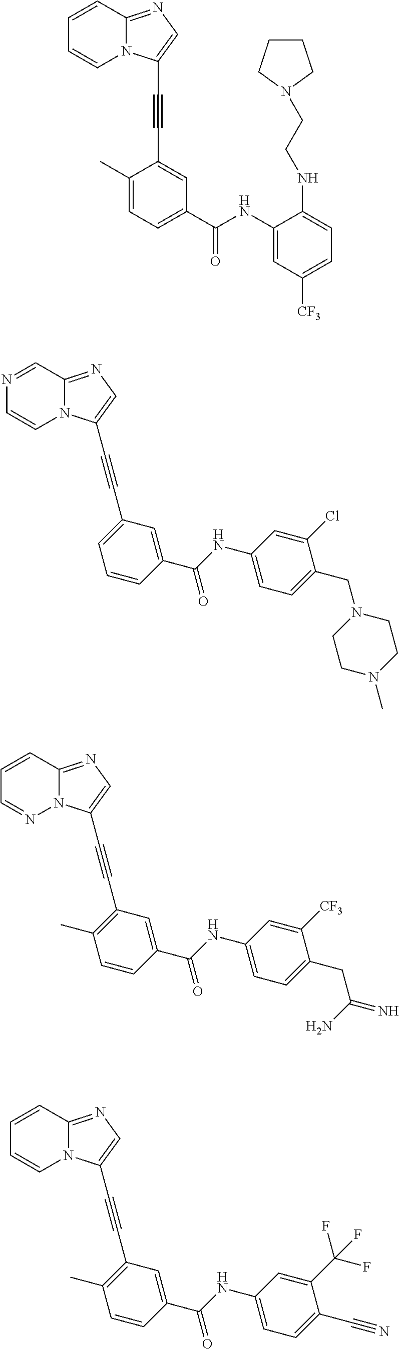 [SMILES: Cc1ccc(C(=O)Nc2cc(C(F)(F)F)ccc2NCCN2CCCC2)cc1C#Cc1cnc2ccccn12 ; CN1CCN(Cc2ccc(NC(=O)c3cccc(C#Cc4cnc5cnccn45)c3)cc2Cl)CC1 ; Cc1ccc(C(=O)Nc2ccc(CC(=N)N)c(C(F)(F)F)c2)cc1C#Cc1cnc2cccnn12 ; Cc1ccc(C(=O)Nc2ccc(C#N)c(C(F)(F)F)c2)cc1C#Cc1cnc2ccccn12] 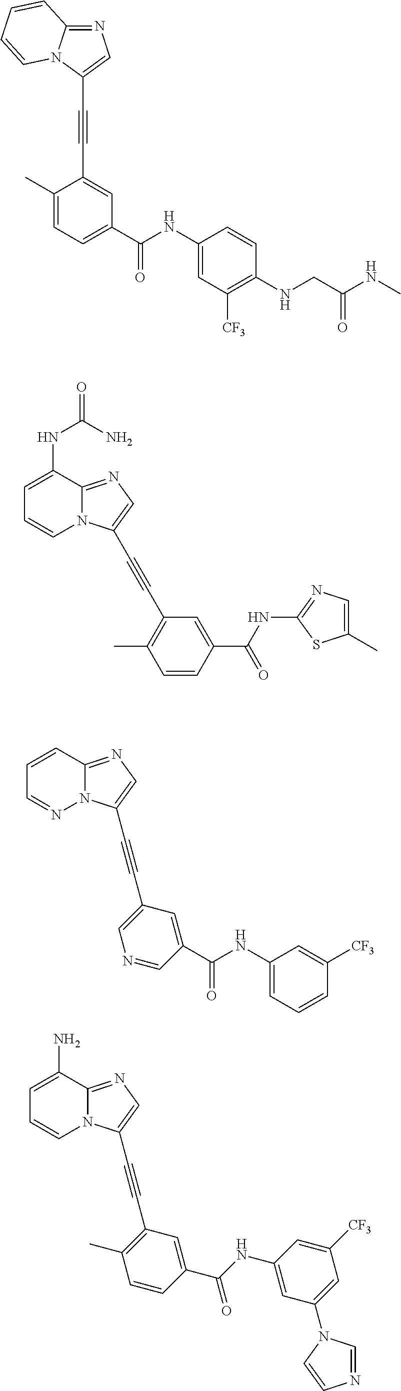 [SMILES: CNC(=O)CNc1ccc(NC(=O)c2ccc(C)c(C#Cc3cnc4ccccn34)c2)cc1C(F)(F)F ; Cc1cnc(NC(=O)c2ccc(C)c(C#Cc3cnc4c(NC(N)=O)cccn34)c2)s1 ; O=C(Nc1cccc(C(F)(F)F)c1)c1cncc(C#Cc2cnc3cccnn23)c1 ; Cc1ccc(C(=O)Nc2cc(-n3ccnc3)cc(C(F)(F)F)c2)cc1C#Cc1cnc2c(N)cccn12] 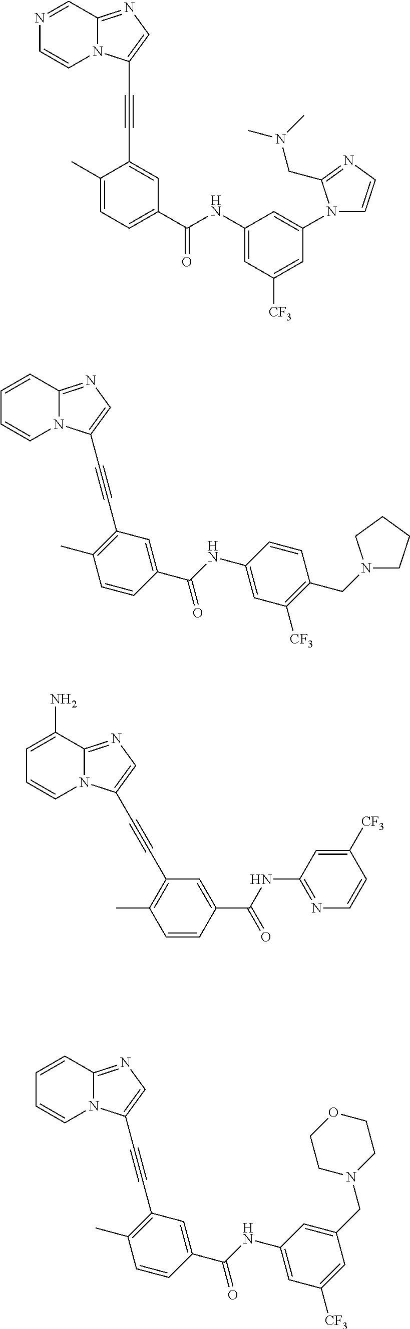 [SMILES: Cc1ccc(C(=O)Nc2cc(-n3ccnc3CN(C)C)cc(C(F)(F)F)c2)cc1C#Cc1cnc2cnccn12 ; Cc1ccc(C(=O)Nc2ccc(CN3CCCC3)c(C(F)(F)F)c2)cc1C#Cc1cnc2ccccn12 ; Cc1ccc(C(=O)Nc2cc(C(F)(F)F)ccn2)cc1C#Cc1cnc2c(N)cccn12 ; Cc1ccc(C(=O)Nc2cc(CN3CCOCC3)cc(C(F)(F)F)c2)cc1C#Cc1cnc2ccccn12] 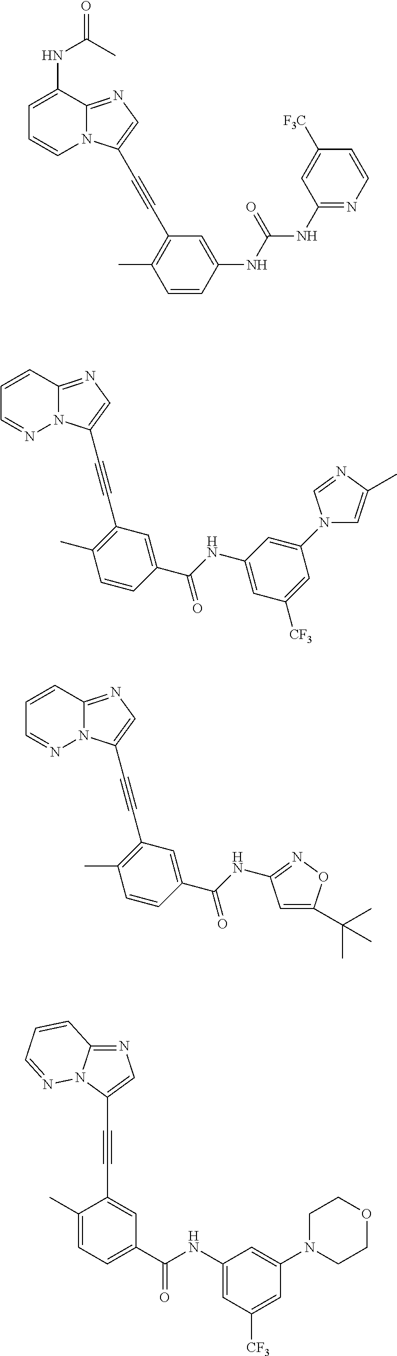 [SMILES: CC(=O)Nc1cccn2c(C#Cc3cc(NC(=O)Nc4cc(C(F)(F)F)ccn4)ccc3C)cnc12 ; Cc1cn(-c2cc(NC(=O)c3ccc(C)c(C#Cc4cnc5cccnn45)c3)cc(C(F)(F)F)c2)cn1 ; Cc1ccc(C(=O)Nc2cc(C(C)(C)C)on2)cc1C#Cc1cnc2cccnn12 ; Cc1ccc(C(=O)Nc2cc(N3CCOCC3)cc(C(F)(F)F)c2)cc1C#Cc1cnc2cccnn12] 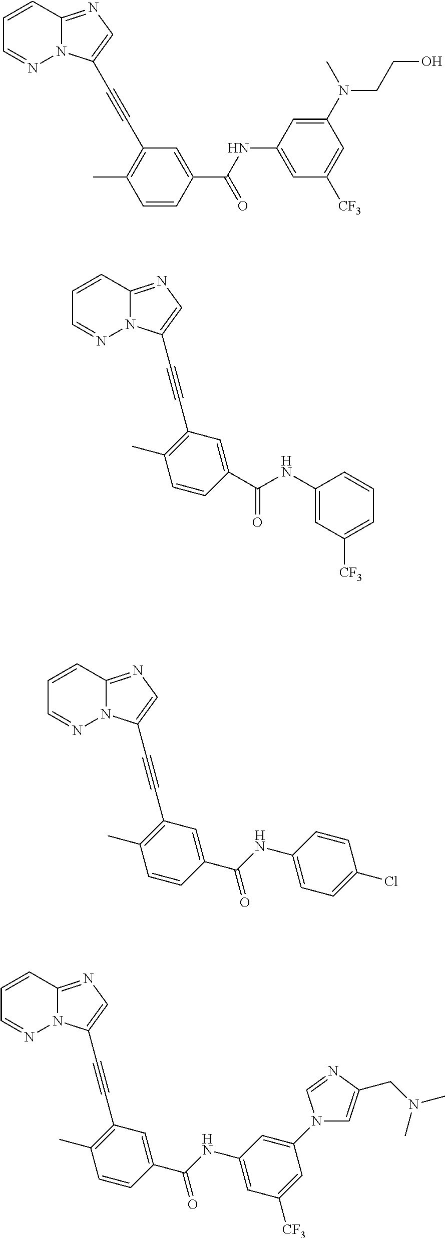 [SMILES: Cc1ccc(C(=O)Nc2cc(N(C)CCO)cc(C(F)(F)F)c2)cc1C#Cc1cnc2cccnn12 ; Cc1ccc(C(=O)Nc2cccc(C(F)(F)F)c2)cc1C#Cc1cnc2cccnn12 ; Cc1ccc(C(=O)Nc2ccc(Cl)cc2)cc1C#Cc1cnc2cccnn12 ; Cc1ccc(C(=O)Nc2cc(-n3cnc(CN(C)C)c3)cc(C(F)(F)F)c2)cc1C#Cc1cnc2cccnn12] 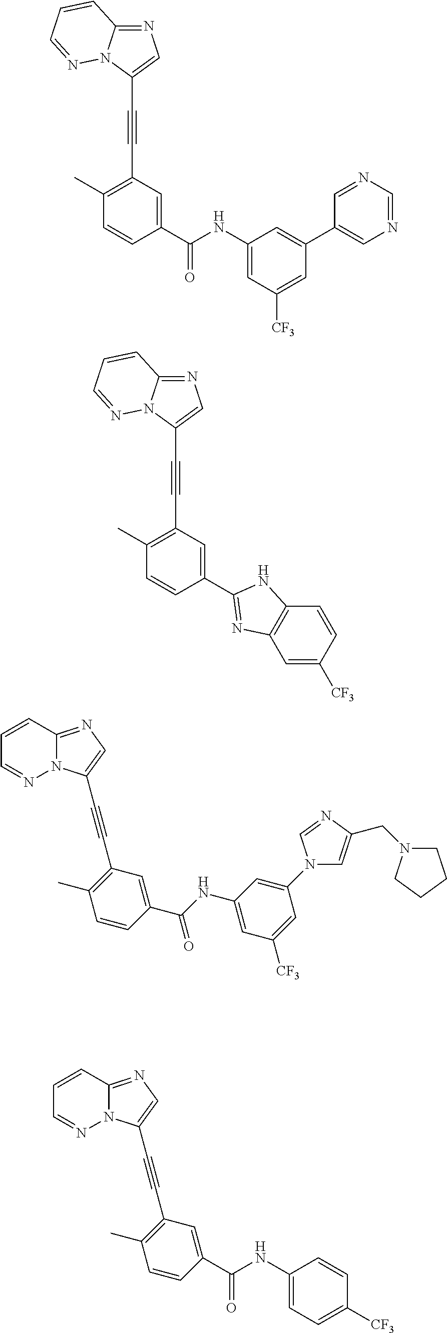 [SMILES: Cc1ccc(C(=O)Nc2cc(-c3cncnc3)cc(C(F)(F)F)c2)cc1C#Cc1cnc2cccnn12 ; Cc1ccc(-c2nc3cc(C(F)(F)F)ccc3[nH]2)cc1C#Cc1cnc2cccnn12 ; Cc1ccc(C(=O)Nc2cc(-n3cnc(CN4CCCC4)c3)cc(C(F)(F)F)c2)cc1C#Cc1cnc2cccnn12 ; Cc1ccc(C(=O)Nc2ccc(C(F)(F)F)cc2)cc1C#Cc1cnc2cccnn12] 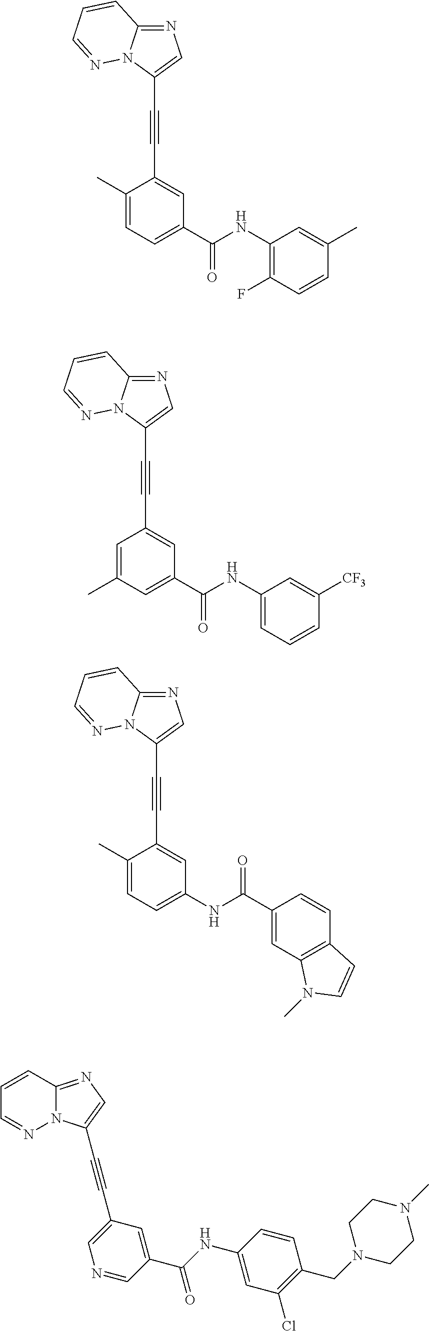 [SMILES: Cc1ccc(F)c(NC(=O)c2ccc(C)c(C#Cc3cnc4cccnn34)c2)c1 ; Cc1cc(C#Cc2cnc3cccnn23)cc(C(=O)Nc2cccc(C(F)(F)F)c2)c1 ; Cc1ccc(NC(=O)c2ccc3ccn(C)c3c2)cc1C#Cc1cnc2cccnn12 ; CN1CCN(Cc2ccc(NC(=O)c3cncc(C#Cc4cnc5cccnn45)c3)cc2Cl)CC1]
or a pharmaceutically acceptable salt thereof.

18. A method according to claim 1, wherein the RAF inhibitor is a compound selected from the group consisting of:
N-(3-(1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyrazin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyrazin-3-ylethynyl)-4-methylbenzamide;
3-(Imidazo[1,2-a]pyridin-3-ylethynyl)-4-methyl-N-(3-(4-methyl-1H-imidazol-1-yl)-5-(trifluoromethyl)phenyl)benzamide;
N-(3-(1H-imidazol-1-yl)-5-(trifluoromethyl)phenyl)-3-(imidazo[1,2-a]pyridin-3-ylethynyl)-4-methylbenzamide;
3-(Imidazo[1,2-a]pyridin-3-ylethynyl)-4-methyl-N-(4-(trifluoromethyl)pyridin-2-yl)benzamide;
N-(5-tert-butylisoxazol-3-yl)-3-(imidazo[1,2-a]pyridin-3-ylethynyl)-4-methylbenzamide;
3-(Imidazo[1,2-a]pyridin-3-ylethynyl)-4-methyl-N-(4-((4-methylpiperazin-1-yl)methyl)-3-(trifluoromethyl)phenyl)benzamide;
N-(3-(2-((dimethylamino)methyl)-1H-imidazol-1-yl)-5-(trifluoromethyl)phenyl)-3-(imidazo[1,2-a]pyridin-3-ylethynyl)-4-methylbenzamide;
3-((8-Acetamidoimidazo[1,2-a]pyridin-3-yl)ethynyl)-4-methyl-N-(4-(trifluoromethyl)pyridin-2-yl)benzamide;
N-(3-(1H-imidazol-1-yl)-5-(trifluoromethyl)phenyl)-3-((8-acetamidoimidazo[1,2-a]pyridin-3-yl)ethynyl)-4-methylbenzamide;
4-Methyl-3-((8-(4-(methylsulfonyl)phenylamino)imidazo[1,2-a]pyridin-3-yl)ethynyl)-N-(4-(trifluoromethyl)pyridin-2-yl)benzamide;
4-methyl-3-((8-(4-sulfamoylphenylamino)imidazo[1,2-a]pyridin-3-yl)ethynyl)-N-(4-(trifluoromethyl)pyridin-2-yl)benzamide;
(R)—N-(4-((3-(Dimethylamino)pyrrolidin-1-yl)methyl)-3-(trifluoromethyl)phenyl)-3-(imidazo[1,2-b]pyridazin-3-ylethynyl)-4-methylbenzamide;
N-(3-(Imidazo[1,2-b]pyridazin-3-ylethynyl)-4-methylphenyl)-4-((4-methylpiperazin-1-yl)methyl)-3-(trifluoromethyl)benzamide;
3-(Imidazo[1,2-b]pyridazin-3-ylethynyl)-4-methyl-N-(4-((4-methylpiperazin-1-yl)methyl)-3-(trifluoromethyl)phenyl)benzamide;
N-(3-Chloro-4-((4-methylpiperazin-1-yl)methyl)phenyl)-3-(imidazo[1,2-b]pyridazin-3-ylethynyl)-4-methylbenzamide;
N-(3-Cyclopropyl-4-((4-methylpiperazin-1-yl)methyl)phenyl)-3-(imidazo[1,2-b]pyridazin-3-ylethynyl)-4-methylbenzamide;
3-(Imidazo[1,2-b]pyridazin-3-ylethynyl)-N-(4-((4-methylpiperazin-1-yl)methyl)-3-(trifluoromethyl)phenyl)benzamide;
N-(4-((4-(2-Hydroxyethyl)piperazin-1-yl)methyl)-3-(trifluoromethyl)phenyl)-3-(imidazo[1,2-b]pyridazin-3-ylethynyl)-4-methylbenzamide; and
3-(Imidazo[1,2-b]pyridazin-3-ylethynyl)-4-methyl-N-(4-(piperazin-1-ylmethyl)-3-(trifluoromethyl)phenyl)benzamide,
or a pharmaceutically acceptable salt thereof.

19. A method according to claim 18, wherein the RAF inhibitor is 3-(Imidazo[1,2-b]pyridazin-3-ylethynyl)-4-methyl-N-(4-((4-methylpiperazin-1-yl)methyl)-3-(trifluoromethyl)phenyl)benzamide or a pharmaceutically acceptable salt thereof.

20. A method according to claim 1, wherein the RAF inhibitor, or a pharmaceutically acceptable salt thereof, is administered orally or intravenously.

21. A method according to claim 1, wherein the effective amount of the RAF inhibitor, or a pharmaceutically acceptable salt thereof, is about 5 mg to about 80 mg.

22. A method according to claim 1, wherein the RAF inhibitor, or a pharmaceutically acceptable salt thereof, is administered to the subject more than one day a week or on average 4 to 7 times every 7 day period.

23. A method according to claim 22, wherein the RAF inhibitor, or a pharmaceutically acceptable salt thereof, is administered to the subject daily.

24. A method according to claim 21, wherein an average daily dose of 5±2 mg, 8±2 mg, 12±3 mg, 15±3 mg, 20±4 mg, 25±5 mg, 30±6 mg, 40±8 mg, 45±9 mg, 50±10 mg, or 55±11 mg of the RAF inhibitor, or a pharmaceutically acceptable salt thereof, is administered to the subject.

25. A pharmaceutical composition for treating or preventing a RAF kinase mediated disease or condition in a subject in need thereof comprising an effective amount of a RAF inhibitor, wherein the RAF inhibitor is a compound of Formula I: 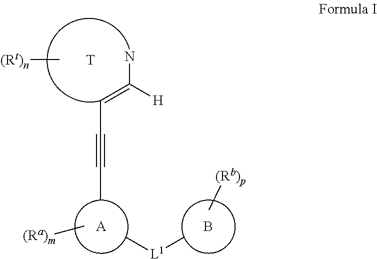
or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;
or a pharmaceutically acceptable salt, solvate or hydrate thereof; and
a pharmaceutically acceptable carrier.

26. A method for the inhibition of a RAF kinase in a subject comprising administering to the subject an effective amount of a compound of Formula I with a RAF kinase, wherein the compound of Formula I is: 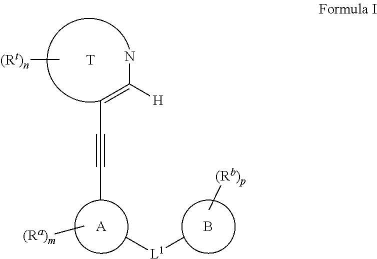
or a tautomer, or an individual isomer or a mixture of isomers thereof wherein:
Ring T is a 5-membered heteroaryl ring containing 1 or 2 nitrogens with the remaining ring atoms being carbon, substituted on at least two ring atoms with R^{t} groups, at least two of which being located on adjacent ring atoms, and, together with the atoms to which they are attached, forming a saturated, partially saturated or unsaturated 5- or 6-membered ring (Ring E), containing 0-3 heteroatoms selected from O, N, and S and being optionally substituted with 1-4 R^{e} groups;
Ring A is a 5- or 6-membered aryl or heteroaryl ring and is optionally substituted with 1-4 R^{a} groups;
Ring B is a 5- or 6-membered aryl or heteroaryl ring;
L¹ is selected from NR¹C(O), C(O)NR¹, NR¹C(O)O, NR¹C(O)NR¹, and OC(O)NR¹;
each occurrence of R^{a}, R^{b} and R^{t} is independently selected from the group consisting of halo, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R^{e}, at each occurrence, is independently selected from the group consisting of halo, ═O, —CN, —NO₂, —R⁴, —OR², —NR²R³, —C(O)YR², —OC(O)YR², —NR²C(O)YR², —SC(O)YR², —NR²C(═S)YR², —OC(═S)YR², —C(═S)YR², —YC(═NR³)YR², —YP(═O)(YR⁴)(YR⁴), —Si(R²)₃, —NR²SO₂R², —S(O)ᵣR², —SO₂NR²R³ and —NR²SO₂NR²R³, wherein each Y is independently a bond, —O—, —S— or —NR³—;
R¹, R² and R³ are independently selected from H, alkyl, alkenyl, alkynyl, cycloalkyl, cycloalkenyl, cycloalkynyl, aryl, heterocyclic and heteroaryl;
alternatively, R² and R³, taken together with the atom to which they are attached, form a 5- or 6-membered saturated, partially saturated or unsaturated ring, which can be optionally substituted and which contains 0-2 heteroatoms selected from N, O and S(O)ᵣ;
each occurrence of R⁴ is independently selected from alkyl, alkenyl, alkynyl, cycloalkyl, cycloalkenyl, cycloalkynyl, aryl, heterocyclic and heteroaryl;
each of the alkyl, alkenyl, alkynyl, cycloalkyl, cycloalkenyl, cycloalkynyl, aryl, heterocyclic and heteroaryl moieties is optionally substituted;
m is 0, 1, 2, 3 or 4;
n is 2 or 3;
p is 0, 1, 2, 3, 4 or 5; and,
r is 0, 1 or 2;
or a pharmaceutically acceptable salt, solvate or hydrate thereof.
